SEQUENCE LISTING (287 sequences)

SEQ ID NO: 1-2 - skipped (2 entries)

SEQ ID NO: 3 - protein, 15 aa, Homo sapiens
EPKSCDKTHTCPPCP

SEQ ID NO: 4 - protein, 12 aa, Homo sapiens
ERKCCVECPPCP

SEQ ID NO: 5 - protein, 12 aa, Homo sapiens
ESKYGPPCPSCP

SEQ ID NO: 6 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 8 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 9 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 15 - protein, 235 aa, synthetic construct
DKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 18 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 21 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 23 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 24 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 25 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 26 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 27 - protein, 232 aa, synthetic construct
DKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 235 aa, synthetic construct; identical to SEQ ID NO: 15

SEQ ID NO: 29 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 32 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 33 - protein, 346 aa, synthetic construct
EVQLLESGGGEVQPGGSLRLSCAASGFTFSDAFMYWVRQAPGKEREWVSSISNRGLKTAYAESVKGRFTISRDNAKNTLYLQMSSLRAEDTAVYYCSRDVDADFRGQGTLVTVKPGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 115 aa, synthetic construct
EVQLLESGGGEVQPGGSLRLSCAASGFTFSDAFMYWVRQAPGKEREWVSSISNRGLKTAYAESVKGRFTISRDNAKNTLYLQMSSLRAEDTAVYYCSRDVDADFRGQGTLVTVKP

SEQ ID NO: 35 - protein, 346 aa, synthetic construct
EVQLLESGGGEVQPGGSLRLSCAASGFTFSDAFMYWVRQAPGKCREWVSSISNRGLKTAYAESVKGRFTISRDNAKNTLYLQMSSLRAEDTAVYYCSRDVDADFRGCGTLVTVKPGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 115 aa, synthetic construct
EVQLLESGGGEVQPGGSLRLSCAASGFTFSDAFMYWVRQAPGKCREWVSSISNRGLKTAYAESVKGRFTISRDNAKNTLYLQMSSLRAEDTAVYYCSRDVDADFRGCGTLVTVKP

SEQ ID NO: 37 - protein, 346 aa, synthetic construct
EVQLLESGGGEVQPGGSLRLSCAASGFTFSDAFMYWVRQAPGKECEWVSSISNRGLKTAYAESVKGRFTISRDNAKNTLYLQMSSLRAEDTAVYYCSRDVDADFCGQGTLVTVKPGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 115 aa, synthetic construct
EVQLLESGGGEVQPGGSLRLSCAASGFTFSDAFMYWVRQAPGKECEWVSSISNRGLKTAYAESVKGRFTISRDNAKNTLYLQMSSLRAEDTAVYYCSRDVDADFCGQGTLVTVKP

SEQ ID NO: 39 - protein, 13 aa, synthetic construct
WVRQAPGKEREWV

SEQ ID NO: 40 - protein, 11 aa, synthetic construct
RGQGTLVTVKP

SEQ ID NO: 41 - protein, 13 aa, synthetic construct
WVRQAPGQGLEWI

SEQ ID NO: 42 - protein, 11 aa, synthetic construct
WGQGTLVTVSS

SEQ ID NO: 43 - protein, 4 aa, Homo sapiens
CPPC

SEQ ID NO: 44 - protein, 4 aa, Homo sapiens
CPSC

SEQ ID NO: 45 - protein, 20 aa, synthetic construct
EPKSCDKTHTCPPCPAPPVA

SEQ ID NO: 46 - protein, 17 aa, synthetic construct
ESKYGPPCPPCPAPPVA

SEQ ID NO: 47 - protein, 15 aa, synthetic construct
CPPCPAPGGGGPSVF

SEQ ID NO: 48 - protein, 14 aa, synthetic construct
CPPCPAPGGGPSVF

SEQ ID NO: 49 - protein, 13 aa, synthetic construct
CPPCPAPGGPSVF

SEQ ID NO: 50 - protein, 12 aa, synthetic construct
CPPCPAPGPSVF

SEQ ID NO: 51 - protein, 115 aa, synthetic construct
EVQLVQSGGGLVQPGGSLTLSCVASGIILSHNEMRWYRQNPGKPRDLVAGITSAAYTYYGDFVKGRFTISRDNARNTAYLQMNSLNPGDTGNYYCEVSDGDNQYWGQGTQATVSS

SEQ ID NO: 52 - protein, 115 aa, synthetic construct
QLQLQESGGGLVQPGGSLTLSCVASGIILSHNEMRWYRQNPGEPRDLVAGITSAAYTYYGDFVKGRFTISRDNAKNTAYLQMNRLSPEDTGNYYCEVSDGDNQYWGQGTQVTVSS

SEQ ID NO: 53 - protein, 113 aa, synthetic construct
QVQLQQSGGGLVQPGGSLSLSCVASGIILSHNEMRWYRQNPGKPRDLVAGITSAAYTYYGDFVKGRFTISRDNAKNTAYLQMDRLNPEDTGNYYCEVSDGDNRYWGQGTQATV

SEQ ID NO: 54 - protein, 115 aa, synthetic construct
QVQLQQSGGGLVQPGGSLTLSCVASGIILSHNEMRWYRQNPGKPRDLVAGITSAAYTYYGDFFKGRFTISRDNAKNTAYLQMNRLNPEDTGNYYCEVSDGDIQYWGQGTQATVSS

SEQ ID NO: 55 - protein, 115 aa, synthetic construct
QVQLVQSGGGLVQPGGSLTLSCAASGIILSHNEMRWYRQNPGKPRDLVAGITGLDYTYYGDFVKGRFTISRDNAMNTAYLQMDSLNPEDTGNYYCEVSDGDNQYWGQGTQVTVSS

SEQ ID NO: 56 - protein, 115 aa, synthetic construct
QVQLQESGGGLVQPGGSLRLSCAASGFTFSDAFMYWVRQAPGKELEWVSSISNRGLKTAYAESVKGRLTISRDNTKNTLYLEMNSLKPEDTGMYYCSRDVGGDLRGQGTQVTVSS

SEQ ID NO: 57 - protein, 113 aa, synthetic construct
QVQLQESGGGLVQPGGSLRLSCAASGFTFSDAFMYWVRQAPGKELEWVSSISNRGLKTAYKESVKGRFTISRDNTKNMLFLEMNRLEPEDTGLYYCSRDVDGDFRGQGTQVTV

SEQ ID NO: 58 - protein, 122 aa, synthetic construct
QVQLQESGGGLVQPGGSLRLSCAASGFTFSDAFMYWVRQAPGKELEWVSSISNRGLKTAYAESVKGRFTISRDNTKNTLYLQMSSLKPEDTAVYYCATESIGGNGSPYFDLWGQGTQVTVKP

SEQ ID NO: 59 - protein, 122 aa, synthetic construct
QVQLQESGGGLVQPGGSLRLSCAASGFTFSDAFMYWVRQAPGKELEWVSSISNRGLKTAYAESVKGRFTISRDNTKNTLYLQMSSLKPEDTAVYYCATESIGSNGSPYFDLWGQGTQVTVKP

SEQ ID NO: 60 - protein, 116 aa, synthetic construct
QVQLQQSGGGLVQPGGSLRLSCAASGFTFSDAFMYWVRQAPGKELEWVSSISNRGLKTAYKESVKGRFTISRDNTKNMLFLEMNRLEPEDTGLYYCVEGDWNLGPRGQGTQVTVKP

SEQ ID NO: 61 - protein, 116 aa, synthetic construct
EVQLLESGGGEVQPGGSLRLSCAASGFTFSDAFMYWVRQAPGKGLEWVSSISNRGLKTAYAESVKGRFTISRDNAKNTLYLQMSSLRAEDTAVYYCVEGDWNLGPRGQGTLVTVKP

SEQ ID NO: 62 - protein, 116 aa, synthetic construct
EVQLLESGGGEVQPGGSLRLSCAASGFTFSDAFMYWVRQAPGKELEWVSSISNRGLKTAYAESVKGRFTISRDNAKNTLYLQMSSLRAEDTAVYYCVEGDWNLGPRGQGTLVTVKP

SEQ ID NO: 63 - protein, 116 aa, synthetic construct
EVQLLESGGGEVQPGGSLRLSCAASGFTFSDAFMYWVRQAPGKELEWVSSISNRGLKTAYAESVKGRFTISRDNTKNTLYLQMSSLRAEDTAVYYCVEGDWNLGPRGQGTLVTVKP

SEQ ID NO: 64 - protein, 116 aa, synthetic construct
EVQLLESGGGEVQPGGSLRLSCAASGFTFSDAFMYWVRQAPGKELEWVSSISNRGLKTAYAESVKGRFTISRDNTKNMLFLEMNRLEPEDTGLYYCVEGDWNLGPRGQGTLVTVKP

SEQ ID NO: 65 - protein, 116 aa, synthetic construct
EVQLLESGGGEVQPGGSLRLSCAASGFTFSDAFMYWVRQAPGKELEWVSSISNRGLKTAYAESVKGRFTISRDNTKNTLFLEMNRLEPEDTGLYYCVEGDWNLGPRGQGTLVTVKP

SEQ ID NO: 66 - protein, 116 aa, synthetic construct
EVQLLESGGGEVQPGGSLRLSCAASGFTFSDAFMYWVRQAPGKELEWVSSISNRGLKTAYKESVKGRFTISRDNTKNMLFLEMNRLEPEDTGLYYCVEGDWNLGPRGQGTLVTVKP

SEQ ID NO: 67 - protein, 116 aa, synthetic construct
EVQLLESGGGEVQPGGSLRLSCAASGFTFSDAFMYWVRQAPGKELEWVSSISNRGLKTAYKESVKGRFTISRDNTKNTLYLQMSSLRAEDTAVYYCVEGDWNLGPRGQGTLVTVKP

SEQ ID NO: 68 - protein, 116 aa, synthetic construct
EVQLLESGGGEVQPGGSLRLSCAASGFTFSDAFMYWVRQAPGKGREWVSSISNRGLKTAYAESVKGRFTISRDNAKNTLYLQMSSLRAEDTAVYYCVEGDWNLGPRGQGTLVTVKP

SEQ ID NO: 69 - protein, 116 aa, synthetic construct
EVQLLESGGGEVQPGGSLRLSCAASGFTFSDAFMYWVRQAPGKEREWVSSISNRGLKTAYAESVKGRFTISRDNAKNTLYLQMSSLRAEDTAVYYCVEGDWNLGPRGQGTLVTVKP

SEQ ID NO: 70 - protein, 115 aa, synthetic construct
QVQLQESGGGLVQPGGSLRLSCAASGFTFSDAFMYWVRQAPGKELEWVSSISNRGLKTAYKESVKGRFTISRDNTKNMLYLEMNRLEPEDTGLYYCSRDVDGGFRGQGTQVTVKP

SEQ ID NO: 71 - protein, 115 aa, synthetic construct
  VARIANT 64: note = Any amino acid
  VARIANT 91: note = Any amino acid
QVQLQESGGGLVQPGGSLRLSCAASGFTFNDAFMYWVRQAPGKELEWVSSISNRGLKTAYKESXKGLFTISRDNTKNMLFLEMNRLEPEDXGLYYCSRDVDGDFWGRGTHVTVKP

SEQ ID NO: 72 - protein, 115 aa, synthetic construct
QVQLQESGGGLVQPGGSLRLSCAASGFTFSDAFMYWVRQAPGKELEWVSSISNRGLKTAYAESVKGRFTISRDNTKNTLYLEMNSLKPEDTGMYYCSRDVGGDLRGQGTQVTVKP

SEQ ID NO: 73 - protein, 115 aa, synthetic construct
EVQLLESGGGEVQPGGSLRLSCAASGFTFSDAFMYWVRQAPGKGLEWVSSISNRGLKTAYAESVKGRFTISRDNAKNTLYLQMSSLRAEDTAVYYCSRDVDGDFRGQGTLVTVKP

SEQ ID NO: 74 - protein, 115 aa, synthetic construct
EVQLLESGGGEVQPGGSLRLSCAASGFTFSDAFMYWVRQAPGKGLEWVSSISNRGLKTAYAESVKGRFTISRDNAKNTLYLQMSSLRAEDTAVYYCSIDVDGDFRGQGTLVTVKP

SEQ ID NO: 75 - protein, 128 aa, synthetic construct
EVQLVESGGGLVQPGGSLRLSCAASGRSFSTYIMGWFRQAPGKEREFVATISRSGITTRSADSVKGRFTISRDNSKNTVYLQMNSLRPEDTAVYYCAAGPYVEQTLGLYQTLGPWDYWGQGTLVTVSS

SEQ ID NO: 76 - protein, 124 aa, synthetic construct
EVQLVESGGGLVQPGGSLRLSCAASGRTFSSIYAKGWFRQAPGKEREFVAAISRSGRSTSYADSVKGRFTISRDNAKNTVYLQMNSLRPEDTAVYYCAAVGGATTVTASEWDYWGLGTLVTVSS

SEQ ID NO: 77 - protein, 124 aa, synthetic construct
EVQLVESGGGLVQPGGSLRLSCAASGRTFSSIYAKGWFRQAPGKEREFVAAISRSGRSTSYADSVKGRFTISRDNSKNTVYLQMNSLRPEDTAVYYCAAVGGATTVTASEWDYWGLGTLVTVSS

SEQ ID NO: 78 - protein, 124 aa, synthetic construct
EVQLVESGGGLVQPGGSLRLSCAASGRTFSSIYAKGWFRQAPGKEREFVAAISRSGRSTSYADSVKGRFTISRDNAKNTVYLQMNSLRPEDTAVYYCAAVGGATTVTASEWDYWGQGTLVTVSS

SEQ ID NO: 79 - protein, 124 aa, synthetic construct
EVQLVESGGGLVQPGGSLRLSCAASGRTFSSIYAKGWFRQAPGKEREFVAAISRSGRSTSYADSVKGRFTISRDNSKNTVYLQMNSLRPEDTAVYYCAAVGGATTVTASEWDYWGQGTLVTVSS

SEQ ID NO: 80 - protein, 124 aa, synthetic construct
DVQLVESGGGLVQPGGSLRLSCAASGRTFSSIYAKGWFRQAPGKEREFVAAISRSGRSTSYADSVKGRFTISRDNAKNTVYLQMNSLRPEDTAVYYCAAVGGATTVTASEWDYWGLGTLVTVSS

SEQ ID NO: 81 - protein, 124 aa, synthetic construct
DVQLVESGGGLVQPGGSLRLSCAASGRTFSSIYAKGWFRQAPGKEREFVAAISRSGRSTSYADSVKGRFTISRDNSKNTVYLQMNSLRPEDTAVYYCAAVGGATTVTASEWDYWGQGTLVTVSS

SEQ ID NO: 82 - protein, 117 aa, synthetic construct
DVQLVESGGGLVQAGGSLRLSCAASRSIGRLDRMGWYRHRTGEPRELVATITGGSSINYGDFVKGRFTISIDNAKNTVYLQMNNLKPEDTAVYYCNFNKYVTSRDTWGQGTQVTVSS

SEQ ID NO: 83 - protein, 117 aa, synthetic construct
DVQLVESGGGLVQPGGSLRLSCAASRSIGRLDRMGWYRHRTGEPRELVATITGGSSINYGDFVKGRFTISRDNSKNTVYLQMNSLRPEDTAVYYCNFNKYVTSRDTWGQGTLVTVSS

SEQ ID NO: 84 - protein, 117 aa, synthetic construct
DVQLVESGGGLVQPGGSLRLSCAASRSIGRLDRMGWYRHATGEPRELVATITGGSSINYGDFVKGRFTISRDNSKNTVYLQMNSLRPEDTAVYYCNFNKYVTSRDTWGQGTLVTVSS

SEQ ID NO: 85 - protein, 117 aa, synthetic construct
DVQLVESGGGLVQPGGSLRLSCAASRSIGRLDRMGWYRHRPGEPRELVATITGGSSINYGDFVKGRFTISRDNSKNTVYLQMNSLRPEDTAVYYCNFNKYVTSRDTWGQGTLVTVSS

SEQ ID NO: 86 - protein, 117 aa, synthetic construct
DVQLVESGGGLVQPGGSLRLSCAASRSIGRLDRMGWYRHRTGKPRELVATITGGSSINYGDFVKGRFTISRDNSKNTVYLQMNSLRPEDTAVYYCNFNKYVTSRDTWGQGTLVTVSS

SEQ ID NO: 87 - protein, 117 aa, synthetic construct
DVQLVESGGGLVQPGGSLRLSCAASRSIGRLDRMGWYRHAPGEPRELVATITGGSSINYGDFVKGRFTISRDNSKNTVYLQMNSLRPEDTAVYYCNFNKYVTSRDTWGQGTLVTVSS

SEQ ID NO: 88 - protein, 117 aa, synthetic construct
DVQLVESGGGLVQPGGSLRLSCAASRSIGRLDRMGWYRHATGKPRELVATITGGSSINYGDFVKGRFTISRDNSKNTVYLQMNSLRPEDTAVYYCNFNKYVTSRDTWGQGTLVTVSS

SEQ ID NO: 89 - protein, 117 aa, synthetic construct
DVQLVESGGGLVQPGGSLRLSCAASRSIGRLDRMGWYRHRPGKPRELVATITGGSSINYGDFVKGRFTISRDNSKNTVYLQMNSLRPEDTAVYYCNFNKYVTSRDTWGQGTLVTVSS

SEQ ID NO: 90 - protein, 117 aa, synthetic construct
DVQLVESGGGLVQPGGSLRLSCAASRSIGRLDRMGWYRHAPGKPRELVATITGGSSINYGDFVKGRFTISRDNSKNTVYLQMNSLRPEDTAVYYCNFNKYVTSRDTWGQGTLVTVSS

SEQ ID NO: 91 - protein, 117 aa, synthetic construct
DVQLVESGGGLVQPGGSLRLSCAASRSIGRLDRMGWYRHRTGEPRELVATITGGSSINYADFVKGRFTISRDNSKNTVYLQMNSLRPEDTAVYYCNFNKYVTSRDTWGQGTLVTVSS

SEQ ID NO: 92 - protein, 117 aa, synthetic construct
DVQLVESGGGLVQPGGSLRLSCAASRSIGRLDRMGWYRHRTGEPRELVATITGGSSINYGDSVKGRFTISRDNSKNTVYLQMNSLRPEDTAVYYCNFNKYVTSRDTWGQGTLVTVSS

SEQ ID NO: 93 - protein, 117 aa, synthetic construct
DVQLVESGGGLVQPGGSLRLSCAASRSIGRLDRMGWYRHRTGEPRELVATITGGSSINYADSVKGRFTISRDNSKNTVYLQMNSLRPEDTAVYYCNFNKYVTSRDTWGQGTLVTVSS

SEQ ID NO: 94 - protein, 117 aa, synthetic construct
DVQLVESGGGLVQPGGSLRLSCAASRSIGRLDRMGWYRHRTGEPRELVATITGGSSINYGDFVKGRFTISIDNSKNTVYLQMNSLRPEDTAVYYCNFNKYVTSRDTWGQGTLVTVSS

SEQ ID NO: 95 - protein, 117 aa, synthetic construct
DVQLVESGGGLVQPGGSLRLSCAASRSIGRLDRMGWYRHRTGEPRELVATITGGSSINYGDFVKGRFTISRDNAKNTVYLQMNSLRPEDTAVYYCNFNKYVTSRDTWGQGTLVTVSS

SEQ ID NO: 96 - protein, 117 aa, synthetic construct
DVQLVESGGGLVQPGGSLRLSCAASRSIGRLDRMGWYRHRTGEPRELVATITGGSSINYGDFVKGRFTISRDNSKNTVYLQMNNLRPEDTAVYYCNFNKYVTSRDTWGQGTLVTVSS

SEQ ID NO: 97 - protein, 117 aa, synthetic construct
DVQLVESGGGLVQPGGSLRLSCAASRSIGRLDRMGWYRHRPGKPRELVATITGGSSINYADSVKGRFTISRDNSKNTVYLQMNSLRPEDTAVYYCNFNKYVTSRDTWGQGTLVTVSS

SEQ ID NO: 98 - protein, 117 aa, synthetic construct
DVQLVESGGGLVQPGGSLRLSCAASRSIGRLDRMGWYRHRPGKPRELVATITGGSSINYADSVKGRFTISIDNSKNTVYLQMNSLRPEDTAVYYCNFNKYVTSRDTWGQGTLVTVSS

SEQ ID NO: 99 - protein, 117 aa, synthetic construct
DVQLVESGGGLVQPGGSLRLSCAASRSIGRLDRMGWYRHRPGKPRELVATITGGSSINYADSVKGRFTISRDNSKNTVYLQMNNLRPEDTAVYYCNFNKYVTSRDTWGQGTLVTVSS

SEQ ID NO: 100 - protein, 117 aa, synthetic construct
DVQLVESGGGLVQPGGSLRLSCAASRSIGRLDRMGWYRHRPGKPRELVATITGGSSINYADSVKGRFTISIDNSKNTVYLQMNNLRPEDTAVYYCNFNKYVTSRDTWGQGTLVTVSS

SEQ ID NO: 101 - protein, 117 aa, synthetic construct
EVQLVESGGGLVQPGGSLRLSCAASRSIGRLDRMGWYRHRPGEPRELVATITGGSSINYGDSVKGRFTISIDNSKNTVYLQMNSLRPEDTAVYYCNFNKYVTSRDTWGQGTLVTVSS

SEQ ID NO: 102 - protein, 117 aa, synthetic construct
EVQLVESGGGLVQPGGSLRLSCAASRSIGRLDRMGWYRHRPGKPRELVATITGGSSINYGDSVKGRFTISIDNSKNTVYLQMNSLRPEDTAVYYCNFNKYVTSRDTWGQGTLVTVSS

SEQ ID NO: 103 - protein, 120 aa, synthetic construct
  VARIANT 10: note = Any amino acid
QVQLQESGGXLVQSGGSLRLSCAASGSVFSIDAMGWYRLAPGKQRELVAVMSSGSPKYADSVKGRFTISRGSARGTVYLQMDSLKPEDTAVYYCYADVATGWGRDASAYWGQGTQVTVSS

SEQ ID NO: 104 - protein, 121 aa, synthetic construct
QVQLQQSGGGLVRAGGSLRLSCVAAGSTFSVNSMAWYRQAPGKERELVAAFTGGSTMNYASSVKGRFTISRGNAAHTVLLQMTNLKPEDTAVYYCNAEVNEGWNADYHDYWGQGTQVTVSS

SEQ ID NO: 105 - protein, 121 aa, synthetic construct
QVQLVQSGGGLVQAGGSLRLSCTASGSIFSINHMAWYRQAPGKQREMVAHITGGASTKYADSVKGRFTISRDSALNTVSLRMNSLKPEDTAVYYCNAEVNEGWNADYYDVWGQGTQVTVSS

SEQ ID NO: 106 - protein, 121 aa, synthetic construct
QVQLQESGGGLVQAGGSLRLSCAASGSVFSIDAMGWYRLAPGQQRELVAVLNGISSAKYADSVKGRFTISGDSAKNAVYLQMDGLKPEDTAVYYCYADVSTGWGRDAHGYWGQGTQVTVSS

SEQ ID NO: 107 - protein, 117 aa, synthetic construct
EVQLVQSGGGLVQPGGSLRLSCAASGNIFSIDAMGWYRQAPGRQRELVAQIPGGPTDSVKGRFTVSGNSAKNTGYLQMNTLKPEDTAVYYCNIVASTSWGSPSKVYWGQGTQATVSS

SEQ ID NO: 108 - protein, 120 aa, synthetic construct
QVQLQESGGGLVQPGGSLRLSCAASGSVFSIDSMSWFRQAPGNERELVALITGGRTTTYADSVKGRFTISRASAPNTVYLQMNSLKPEDTAVYYCNAVVSTGWGRNADDYWGQGTQVTVS

SEQ ID NO: 109 - protein, 119 aa, synthetic construct
QVQLQQSGGGLVQAGGSLRLSCAASGSIFSIDAMGWYRLAPGKQRELVAVIDGVSPNYADSVKGRFTISSDIAKNTVYLQMHSPKPEDTAVYYCNADVSTGWGRPADHYWGQGTQVTVS

SEQ ID NO: 110 - protein, 120 aa, synthetic construct
QVQLQESGGGLVQPGGSLRLSCAASGSVFSIDSMSWFRQAPGNERELVALITGGHTTTYGDSVKGRFTISRASAPNTVHLQMNSLQPEDTAVYYCNAAVSTGWGRNADDYWGQGTQVTVS

SEQ ID NO: 111 - protein, 118 aa, synthetic construct
QLVQSGGGLVQPGESLRLSCAASGSVFSIDSVSWFRQGPGNERELVALITGGRTTTYADSVKGRFTISRANAPNTVHLRMNSLKPEDTAVYYCNAAVSTGWGRNADDYWGQGTQVTVS

SEQ ID NO: 112 - protein, 120 aa, synthetic construct
QVQLVQSGGGLVQPGGSLRLICAASGSVFSIDSMSWFRQRPGNERELVALITGGRTTTYSDSVKGRFTISRASALNTVHLQMNSLKPEDTAVYYCNAALSTGWGRDASAYWGQGTQVTVS

SEQ ID NO: 113 - protein, 120 aa, synthetic construct
QVQLQESGGGLVQAGGSLRLSCTASGSIFSINHMAWYRQAPGKQREMVAHITGGASTKYADSVKGRFTISRDSALNTVSLRMNSLKPEDTAVYYCNAEVNEGWNADYYDVWGQGTQVTVS

SEQ ID NO: 114 - protein, 119 aa, synthetic construct
QLQLQESGGGTVQAGGSLRLSCAASRSIASINVMGWYRQAPGNQHELVAAITSGGSPNYAGSVRGRFIISRDNAKNTVYLQMNDLKPEDTAVYYCAGELRDDSNGYLHYWGQGTQVTVS

SEQ ID NO: 115 - protein, 120 aa, synthetic construct
QVQLQESGGGLVQPGGSLRLSCAASGSVFSIDSMSWFRQTPGNERELVAHITGGRTTTYADSVKGRFTISRASAPNTVHLQMNNLKPEDTAVYYCNAAVSTGWGRNADDYWGQGTQVTVS

SEQ ID NO: 116 - protein, 119 aa, synthetic construct
QVQLQESGGGLVQAGGSLRLSCTASGSIFSIDDMGWYRLAPGKQRELVAVHSGSSTNYGDSVKGRFTISGDSAKNTVYLQMHRLEPEDTAVYYCYAAISSGWGRDAEDYWGQGTQVTVS

SEQ ID NO: 117 - protein, 120 aa, synthetic construct
QVQLVQSGGGLVQPGESLRLSCAASGSVFSIDSMSWFRQGPGNERELVALITGGRTTTYADSVKGRFTISRANAPNTVHLQMNSLKPEDTAVYYCNAAVSTGWGRSADDYWGQGTQVTVS

SEQ ID NO: 118 - protein, 120 aa, synthetic construct
QVQLVQSGGGLVQPGESLRLSCAASGSVFSIDSMSWFRQGPGNERELVALITGGRTTTYADSVKGRFTISRANAPNTVHLQMNSLEPEDTAVYYCNAAVSTGWGRNADDYWGQGTQVTVS

SEQ ID NO: 119 - protein, 120 aa, synthetic construct
QVQLVQSGGGLVQPGGSLRLSCAASGSVFSIDSMSWFRQAPGNERELVALITGGRTTTYADSVKGRFTISRASAPNTVHLQMNSLKPEDTAVYYCNAVVSTGWGRNADDYWGQGTQVTVS

SEQ ID NO: 120 - protein, 121 aa, synthetic construct
EVQLLESGGGEVQPGGSLRLSCAASGSVFSIDAMSWFRQAPGKGLELVSAITGGRTTYYAESVKGRFTISRDNAKNTLYLQMSSLRAEDTAVYYCNAVVSTGWGRNADDYWGQGTLVTVKP

SEQ ID NO: 121 - protein, 121 aa, synthetic construct
EVQLLESGGGEVQPGGSLRLSCAASGSVFSIDAMSWFRQAPGKGLELVCAITGGRTTYYAESVKGRFTCSRDNAKNTLYLQMSSLRAEDTAVYYCNAVVSTGWGRNADDYWGQGTLVTVKP

SEQ ID NO: 122 - protein, 121 aa, synthetic construct
EVQLLESGGGEVQPGGSLRLSCAASGSVFSIDSMSWFRQAPGKGLELVSLITGGRTTYYAESVKGRFTISRDNAKNTLYLQMSSLRAEDTAVYYCNAVVSTGWGRNADDYWGQGTLVTVKP

SEQ ID NO: 123 - protein, 121 aa, synthetic construct
EVQLLESGGGEVQPGGSLRLSCAASGSVFSIDSMSWFRQAPGKGLELVCLITGGRTTYYAESVKGRFTCSRDNAKNTLYLQMSSLRAEDTAVYYCNAVVSTGWGRNADDYWGQGTLVTVKP

SEQ ID NO: 124 - protein, 121 aa, synthetic construct
EVQLLESGGGEVQPGGSLRLSCAASGSVFSIDAMSWFRQAPGKGLELVSLITGGRTTYYAESVKGRFTISRDNAKNTLYLQMSSLRAEDTAVYYCNAVVSTGWGRNADDYWGQGTLVTVKP

SEQ ID NO: 125 - protein, 121 aa, synthetic construct
EVQLLESGGGEVQPGGSLRLSCAASGSVFSIDAMSWFRQAPGKGLELVCLITGGRTTYYAESVKGRFTCSRDNAKNTLYLQMSSLRAEDTAVYYCNAVVSTGWGRNADDYWGQGTLVTVKP

SEQ ID NO: 126 - protein, 119 aa, synthetic construct
EVQLLESGGGEVQPGGSLRLSCAASGSVFSIDAMGWYRQAPGKGLELVSALSGISSATYAESVKGRFTISRDNAKNTLYLQMSSLRAEDTAVYYCYADVSTGWGRDAHGYWGQGTLVTV

SEQ ID NO: 127 - protein, 119 aa, synthetic construct
EVQLLESGGGEVQPGGSLRLSCAASGSVFSIDAMGWYRQAPGKGRELVSALSGISSATYAESVKGRFTISRDNAKNTLYLQMSSLRAEDTAVYYCYADVSTGWGRDAHGYWGQGTLVTV

SEQ ID NO: 128 - protein, 119 aa, synthetic construct
EVQLLESGGGEVQPGGSLRLSCAASGSVFSIDAMGWYRQAPGKQRELVSALSGISSATYAESVKGRFTISRDNAKNTLYLQMSSLRAEDTAVYYCYADVSTGWGRDAHGYWGQGTLVTV

SEQ ID NO: 129 - protein, 119 aa, synthetic construct
EVQLLESGGGEVQPGGSLRLSCAASGSVFSIDAMGWYRQAPGQQRELVSALSGISSATYAESVKGRFTISRDNAKNTLYLQMSSLRAEDTAVYYCYADVSTGWGRDAHGYWGQGTLVTV

SEQ ID NO: 130 - protein, 119 aa, synthetic construct
EVQLLESGGGEVQPGGSLRLSCAASGSVFSIDAMGWYRQAPGKGLELVAVLSGISSATYAESVKGRFTISRDNAKNTLYLQMSSLRAEDTAVYYCYADVSTGWGRDAHGYWGQGTLVTV

SEQ ID NO: 131 - protein, 119 aa, synthetic construct
EVQLLESGGGEVQPGGSLRLSCAASGSVFSIDAMGWYRQAPGKGRELVAVLSGISSATYAESVKGRFTISRDNAKNTLYLQMSSLRAEDTAVYYCYADVSTGWGRDAHGYWGQGTLVTV

SEQ ID NO: 132 - protein, 119 aa, synthetic construct
EVQLLESGGGEVQPGGSLRLSCAASGSVFSIDAMGWYRQAPGKQRELVAVLSGISSATYAESVKGRFTISRDNAKNTLYLQMSSLRAEDTAVYYCYADVSTGWGRDAHGYWGQGTLVTV

SEQ ID NO: 133 - protein, 119 aa, synthetic construct
EVQLLESGGGEVQPGGSLRLSCAASGSVFSIDAMGWYRQAPGQQRELVAVLSGISSATYAESVKGRFTISRDNAKNTLYLQMSSLRAEDTAVYYCYADVSTGWGRDAHGYWGQGTLVTV

SEQ ID NO: 134 - protein, 119 aa, synthetic construct
EVQLLESGGGEVQPGGSLRLSCAASGSVFSIDAMGWYRQAPGKQRELVAVLSGISSAKYAESVKGRFTISRDNAKNTLYLQMSSLRAEDTAVYYCYADVSTGWGRDAHGYWGQGTLVTV

SEQ ID NO: 135 - protein, 119 aa, synthetic construct
EVQLLESGGGEVQPGGSLRLSCAASGSVFSIDAMGWYRLAPGQQRELVAVLSGISSAKYAESVKGRFTISRDNAKNTLYLQMSSLRAEDTAVYYCYADVSTGWGRDAHGYWGQGTLVTV

SEQ ID NO: 136 - protein, 119 aa, synthetic construct
EVQLLESGGGEVQPGGSLRLSCAASGSVFSIDAMGWYRQAPGKQRELVAVLSGISSAKYADSVKGRFTISGDNAKNTLYLQMSSLRAEDTAVYYCYADVSTGWGRDAHGYWGQGTLVTV

SEQ ID NO: 137 - protein, 119 aa, synthetic construct
EVQLLESGGGEVQPGGSLRLSCAASGSVFSIDAMGWYRQAPGKQRELVAVLSGISSAKYAESVKGRFTISRDSAKNAVYLQMDGLKPEDTAVYYCYADVSTGWGRDAHGYWGQGTLVTV

SEQ ID NO: 138 - protein, 119 aa, synthetic construct
EVQLLESGGGEVQPGGSLRLSCAASGSVFSIDAMGWYRQAPGKQRELVAVLSGISSAKYAESVKGRFTISRDNAKNTVYLQMSSLRAEDTAVYYCYADVSTGWGRDAHGYWGQGTLVTV

SEQ ID NO: 139 - protein, 119 aa, synthetic construct
EVQLLESGGGEVQPGGSLRLSCAASGSVFSIDAMGWYRQAPGKQRELVAVLSGISSAKYAESVKGRFTISRASAPNTLYLQMSSLRAEDTAVYYCYADVSTGWGRDAHGYWGQGTLVTV

SEQ ID NO: 140 - protein, 119 aa, synthetic construct
EVQLLESGGGEVQPGGSLRLSCAASGSVFSIDAMGWYRQAPGKQRELVAVLSGISSAKYAESVKGRFTISRASAPNTVYLQMSSLRAEDTAVYYCYADVSTGWGRDAHGYWGQGTLVTV

SEQ ID NO: 141 - protein, 119 aa, synthetic construct
EVQLLESGGGEVQPGGSLRLSCAASGSVFSIDAMGWYRQAPGKQRELVAVLSGISSAKYAASAPGRFTISRDAVKNTVYLQMSSLRAEDTAVYYCYADVSTGWGRDAHGYWGQGTLVTV

SEQ ID NO: 142 - protein, 119 aa, synthetic construct
EVQLLESGGGEVQPGGSLRLSCAASGSVFSIDAMGWYRQAPGKQRELVAVLSGISSAKYAASAPGRFTISRDAVENTVYLQMSSLRAEDTAVYYCYADVSTGWGRDAHGYWGQGTLVTV

SEQ ID NO: 143 - protein, 119 aa, synthetic construct
EVQLLESGGGEVQPGGSLRLSCAASGSVFSIDAMGWYRQAPGKQRELVAVLSGISSAKYAASAPGRFTISRDNAKNTVYLQMSSLRAEDTAVYYCYADVSTGWGRDAHGYWGQGTLVTV

SEQ ID NO: 144 - protein, 119 aa, synthetic construct
EVQLLESGGGEVQPGGSLRLSCAASGSVFSIDAMGWYRQAPGKQRELVAVLSGISSAKYADAVKGRFTISRASAPNTVYLQMSSLRAEDTAVYYCYADVSTGWGRDAHGYWGQGTLVTV

SEQ ID NO: 145 - protein, 119 aa, synthetic construct
EVQLLESGGGEVQPGGSLRLSCAASGSVFSIDAMGWYRQAPGKQRELVAVLSGISSAKYADAVEGRFTISRASAPNTVYLQMSSLRAEDTAVYYCYADVSTGWGRDAHGYWGQGTLVTV

SEQ ID NO: 146 - protein, 119 aa, synthetic construct
EVQLLESGGGEVQPGGSLRLSCAASGSVFSIDAMGWYRQAPGKQRELVAVLSGISSAKYAASAPGRFTISRASAPNTVYLQMSSLRAEDTAVYYCYADVSTGWGRDAHGYWGQGTLVTV

SEQ ID NO: 147 - protein, 110 aa, synthetic construct
EVQLLESGGGEVQPGGSLRLSCAASGFTFSTHGMDWFRQAPGKDLEWVSAINNGGSWTSYASSVKGRFTISRDNAKNTLYLQMSSLRAEDTAVYWCQNRVTRGQGTLVTV

SEQ ID NO: 148 - protein, 110 aa, synthetic construct
EVQLLESGGGEVQPGGSLRLSCAASGFTFSTHGMDWFRQAPGKDLEWVSAINNGGSWTSYASSVKGRFTISRDNAKNTLYLQMSSLRAEDTAVYYCQNRVTRGQGTLVTV

SEQ ID NO: 149 - protein, 110 aa, synthetic construct
EVQLLESGGGEVQPGGSLRLSCAASGFTFSTHGMDWFRQAPGKDLEWVSAINQGGSWTSYASSVKGRFTISRDNAKNTLYLQMSSLRAEDTAVYYCQNRVTRGQGTLVTV

SEQ ID NO: 150 - protein, 110 aa, synthetic construct
EVQLLESGGGEVQPGGSLRLSCAASGFTFSTHGMDWFRQAPGKGLEWVSAINNGGSWTSYASSVKGRFTISRDNAKNTLYLQMSSLRAEDTAVYWCQNRVTRGQGTLVTV

SEQ ID NO: 151 - protein, 110 aa, synthetic construct
EVQLLESGGGEVQPGGSLRLSCAASGFTFSTHGMDWFRQAPGKGLEWVSAINNGGSWTSYASSVKGRFTISRDNAKNTLYLQMSSLRAEDTAVYYCQNRVTRGQGTLVTV

SEQ ID NO: 152 - protein, 110 aa, synthetic construct
EVQLLESGGGEVQPGGSLRLSCAASGFTFSTHGMDWFRQAPGKDLEWVSAIQSGGSWTSYASSVKGRFTISRDNAKNTLYLQMSSLRAEDTAVYWCQNRVTRGQGTLVTV

SEQ ID NO: 153 - protein, 110 aa, synthetic construct
EVQLLESGGGEVQPGGSLRLSCAASGFTFSTHGMDWFRQAPGKDLEWVSAIQSGGSWTSYASSVKGRFTISRDNAKNTLYLEMNNLKPEDTAVYWCQNRVTRGQGTLVTV

SEQ ID NO: 154 - protein, 110 aa, synthetic construct
EVQLLESGGGEVQPGGSLRLSCAASGFTFSTHGMDWFRQAPGKDLEWVSAIQSGGSWTSYASSVKGRFTISRDNAKNTLYLQMNNLRAEDTAVYWCQNRVTRGQGTLVTV

SEQ ID NO: 155 - protein, 110 aa, synthetic construct
EVQLLESGGGEVQPGGSLRLSCAASGFTFSTHGMDWFRQAPGKDLEWVSAIQSGGSWTSYASSVKGRFTISRDNAKNTLYLEMSSLRAEDTAVYWCQNRVTRGQGTLVTV

SEQ ID NO: 156 - protein, 110 aa, synthetic construct
EVQLLESGGGEVQPGGSLRLSCAASGFTFSTHGMDWFRQAPGKDLEWVSAIQSGGSWTSYASSVKGRFTISRDNAKNTLYLQMSSLRPEDTAVYWCQNRVTRGQGTLVTV

SEQ ID NO: 157 - protein, 110 aa, synthetic construct
EVQLLESGGGEVQPGGSLRLSCAASGFTFSTHGMDWFRQAPGKDLEWVSAIQSGGSWTSYASSVKGRFTISRDNAKNTLYLQMQQLRAEDTAVYWCQNRVTRGQGTLVTV

SEQ ID NO: 158 - protein, 110 aa, synthetic construct
EVQLLESGGGEVQPGGSLRLSCAASGFTFSTHGMDWFRQAPGKDLEWVSAIQSGGSWTSYASSVKGRFTISRDNAKNTLYLQMQNLRAEDTAVYWCQNRVTRGQGTLVTV

SEQ ID NO: 159 - protein, 110 aa, synthetic construct
EVQLLESGGGEVQPGGSLRLSCAASGFTFSTHGMDWFRQAPGKDLEWVSAIQSGGSWTSYASSVKGRFTISRDNAKNTLYLQMDNLRAEDTAVYWCQNRVTRGQGTLVTV

SEQ ID NO: 160 - protein, 110 aa, synthetic construct
EVQLLESGGGEVQPGGSLRLSCAASGFTFSTHGMDWFRQAPGKDLEWVSAIQSGGSWTSYASSVKGRFTISRDNAKNTLYLQMNDLRAEDTAVYWCQNRVTRGQGTLVTV

SEQ ID NO: 161 - protein, 110 aa, synthetic construct
EVQLLESGGGEVQPGGSLRLSCAASGFTFSTHGMDWFRQAPGKDLEWVSAIQSGGSWTSYASSVKGRFTISRDNAKNTLYLQMDDLRAEDTAVYWCQNRVTRGQGTLVTV

SEQ ID NO: 162 - protein, 110 aa, synthetic construct
EVQLLESGGGEVQPGGSLRLSCAASGFTFSTHGMDWFRQAPGKDLEWVSAIQSGGSWTSYASSVKGRFTISRDNAKNTLYLQMQDLRAEDTAVYWCQNRVTRGQGTLVTV

SEQ ID NO: 163 - protein, 110 aa, synthetic construct
EVQLLESGGGEVQPGGSLRLSCAASGFTFSTHGMDWFRQAPGKDLEWVSAIQSGGSWTSYASSVKGRFTISRDNAKNTLYLQMNQLRAEDTAVYWCQNRVTRGQGTLVTV

SEQ ID NO: 164 - protein, 110 aa, synthetic construct
EVQLLESGGGEVQPGGSLRLSCAASGFTFSTHGMDWFRQAPGKDLEWVSAIQSGGSWTSYASSVKGRFTISRDNAKNTLYLQMSNLRAEDTAVYWCQNRVTRGQGTLVTV

SEQ ID NO: 165 - protein, 126 aa, synthetic construct
QVQLQESGGGLVQAGGSLRLSCAASGRTFGRSAMGWFRQAPGKEREFVAAIGTRGGSTKYADSVKGRFTISTDNASNTVYLQMDSLKPEDTAVYRCAVRGPGYPSAAIFQDEYHYWGQGTQVTVSS

SEQ ID NO: 166 - protein, 156 aa, synthetic construct
QVQLQESGGGLVQAGGSLRLSCAASGRTFGRSAMGWFRQAPGKEREFVAAIGTRGGSTKYADSVKGRFTISTDNASNTVYLQMDSLKPEDTAVYRCAVRGPGYPSAAIFQDEYHYWGQGTQVTVSSGLEGHSDHMEQKLISEEDLNRISDHHHHHH

SEQ ID NO: 167 - protein, 121 aa, synthetic construct
EVQLQESGGGLVQAGGSLRLSCVTSGSAFSSDTMGWFRQAPGKQRELVASISSRGVREYADSVKGRFTISRDNAKNTVYLQMNSLQPEDTAVYYCNRGALGLPGYRPYNNWGQGTQVTVSS

SEQ ID NO: 168 - protein, 151 aa, synthetic construct
EVQLQESGGGLVQAGGSLRLSCVTSGSAFSSDTMGWFRQAPGKQRELVASISSRGVREYADSVKGRFTISRDNAKNTVYLQMNSLQPEDTAVYYCNRGALGLPGYRPYNNWGQGTQVTVSSGLEGHSDHMEQKLISEEDLNRISDHHHHHH

SEQ ID NO: 169 - protein, 112 aa, synthetic construct
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSS

SEQ ID NO: 170 - protein, 142 aa, synthetic construct
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGLEGHSDHMEQKLISEEDLNRISDHHHHHH

SEQ ID NO: 171 - protein, 123 aa, synthetic construct
QVQLQESGGGLVQAGDSLRLSCAASGWAFDNYGMAWFRQAPGKEREFIGRLAWNGGSTDYADSVKGRFTISRDNPKNTLYLQMNNLKPEDTAVYYCARQRSYSGYGIRTPQTYDYWGQGTQVT

SEQ ID NO: 172 - protein, 123 aa, synthetic construct
QVQLQQSGGGLVQAGDSLRLSCAASGWAFGNYGMAWFRRAPGKEREFIGRLAWNGGSTDYVDSVKGRFTISRDNPKNTLYLQMNNLKPDDTAVYYCARQRSYSRYDIRTPQTYDYWGQGTQVT

SEQ ID NO: 173 - protein, 123 aa, synthetic construct
QVQLVQSGGGLVQPGGSLRLSCAASGWAFDNYGMAWFRQAPGKEREFIGRLAWNGGSTDYADSVKGRFTISRDNPKNTLYLQMNSLKPEDTAVYYCARQRSYSRYGIRAPQTYDYWGQGTQVT

SEQ ID NO: 174 - protein, 118 aa, synthetic construct
QVQLQESGGGLVQPGGSLRLSCAVSGFSFSINAMGWYRQAPGKRREFLAAIDSGRNTVYAVSVKGRFTISRDNAKNTVYLQMNSLKPEDTAIYYCGLLKGNRVVSPSVAYWGQGTQVT

SEQ ID NO: 175 - protein, 120 aa, synthetic construct
EVQPVQSGGGLVQAGESLRLSCAASATIFSNNAMGWYRQAPGKQRELVATITTGGFTNYRDSVKGRFDISRDNAKNTVYLQMNNLKPEDTAVYYCNVVLRYSRDYSYTTVKEYWGQGTQV

SEQ ID NO: 176 - protein, 117 aa, synthetic construct
QVQLVQSGGGLVQPGGSLRLSCAASGFTFSSYAMSWVRQAPGKGLEWVSAIPAGDGSTKYADSVKGRFTISRDNAKNTVYLQMDSLKPEDTAVYFCAKSRGWSTVDDMDYWGKGTQV

SEQ ID NO: 177 - protein, 117 aa, synthetic construct
QVQLVQSGGGLVQPGGSLRLSCVVSGFTFRSYAMSWVRQAPGKGLEWVSTINSGESSTKYADSVKGRFTISRDDAKNTLYLQMSDLKPEDTAVYFCAKHRGWSTVDDINYWGKGTQV

SEQ ID NO: 178 - protein, 119 aa, synthetic construct
QVQLVQSGGGLVQPGGSLRLSCAASGFTFDDHAMSWVRQAPGKGLEWVSAISWNGHYTYYAESMKGRFAISRDNAKNTLYLQMNSLKSEDTAVYYCVKGWRGSYTRDRPFASWGQGTQV

SEQ ID NO: 179 - protein, 118 aa, synthetic construct
EVQLVQSGGGLVQPGGSLRLSCAASGFTFSSYYMSWVRQAPGKGLEWVSTISTNTGGGSTYYAYADSVKGRFTISRDNAKNTLYLEMNSLKPEDTAQYYCVRTRWEGVYDYWGLGTQV

SEQ ID NO: 180 - protein, 126 aa, synthetic construct
EVQLLESGGGEVQPGGSLRLSCAASGWAFGNYGMAWFRQAPGKGLEWVARLAWNGGSTDYAESVKGRFTISRDNAKNTLYLQMSSLRAEDTAVYYCARQRSYSRYDIRTPQTYDYWGQGTLVTVKP

SEQ ID NO: 181 - protein, 126 aa, synthetic construct
EVQLLESGGGEVQPGGSLRLSCAASGWAFGNYGMAWFRQAPGKGREFVARLAWNGGSTDYAESVKGRFTISRDNAKNTLYLQMSSLRAEDTAVYYCARQRSYSRYDIRTPQTYDYWGQGTLVTVKP

SEQ ID NO: 182 - protein, 126 aa, synthetic construct
EVQLLESGGGEVQPGGSLRLSCAASGWAFGNYGMAWFRQAPGKEREFVSRLAWNGGSTDYVAESVKGRFTISRDNAKNTLYLQMSSLRAEDTAVYYCARQRSYSRYDIRTPQTYDYWGQGTLVTVK

SEQ ID NO: 183 - protein, 126 aa, synthetic construct
EVQLLESGGGEVQPGGSLRLSCAASGWAFGNYGMAWFRQAPGKEREFIGRLAWNGGSTDYVESVKGRFTISRDNPKNTLYLQMSSLRAEDTAVYYCARQRSYSRYDIRTPQTYDYWGQGTLVTVKP

SEQ ID NO: 184 - protein, 126 aa, synthetic construct
EVQLLESGGGEVQPGGSLRLSCAASGWAFGNYGMAWFRQAPGKEREFVSRLAWNGGSTDYVESVKGRFTISRDNAKNTLYLQMSSLRAEDTAVYYCARQRSYSRYDIRTPQTYDYWGQGTLVTVKP

SEQ ID NO: 185 - protein, 126 aa, synthetic construct
EVQLLESGGGEVQPGGSLRLSCAASGWAFGNYGMAWFRQAPGKEREFVSRLAWNGGSTDYVESVKGRFTISRDNPKNTLYLQMSSLRAEDTAVYYCARQRSYSRYDIRTPQTYDYWGQGTLVTVKP

SEQ ID NO: 186 - protein, 126 aa, synthetic construct
EVQLLESGGGEVQPGGSLRLSCAASGWAFGNYGMAWFRQAPGKEREFIGRLAWNGGSTDYVESVKGRFTISRDNAKNTLYLQMSSLRAEDTAVYYCARQRSYSRYDIRTPQTYDYWGQGTLVTVKP

SEQ ID NO: 187 - protein, 126 aa, synthetic construct
EVQLLESGGGEVQPGGSLRLSCAASGWAFGNYGMAWFRQAPGKEREFIGRLAWQGGSTDYVESVKGRFTISRDNPKNTLYLQMSSLRAEDTAVYYCARQRSYSRYDIRTPQTYDYWGQGTLVTVKP

SEQ ID NO: 188 - protein, 126 aa, synthetic construct
EVQLLESGGGEVQPGGSLRLSCAASGWAFGNYGMAWFRQAPGKEREFIGRLAWNAGSTDYVESVKGRFTISRDNPKNTLYLQMSSLRAEDTAVYYCARQRSYSRYDIRTPQTYDYWGQGTLVTVKP

SEQ ID NO: 189 - protein, 126 aa, synthetic construct
EVQLLESGGGEVQPGGSLRLSCAASGWAFGNYGMAWFRQAPGKEREFVSRLAWQGGSTDYVESVKGRFTISRDNAKNTLYLQMSSLRAEDTAVYYCARQRSYSRYDIRTPQTYDYWGQGTLVTVKP

SEQ ID NO: 190 - protein, 126 aa, synthetic construct
EVQLLESGGGEVQPGGSLRLSCAASGWAFGNYGMAWFRQAPGKEREFVSRLAWNAGSTDYVESVKGRFTISRDNAKNTLYLQMSSLRAEDTAVYYCARQRSYSRYDIRTPQTYDYWGQGTLVTVKP

SEQ ID NO: 191 - protein, 126 aa, synthetic construct
EVQLLESGGGEVQPGGSLRLSCAASGWAFGNYGMAWFRQAPGKEREFVSRLAWGGGSTDYVESVKGRFTISRDNAKNTLYLQMSSLRAEDTAVYYCARQRSYSRYDIRTPQTYDYWGQGTLVTVKP

SEQ ID NO: 192 - protein, 126 aa, synthetic construct
EVQLLESGGGEVQPGGSLRLSCAASGWAFSNYGMAWFRQAPGKEREFVSRLAWGGGSTDYVESVKGRFTISRDNAKNTLYLQMSSLRAEDTAVYYCARQRSYSRYDIRTPQTYDYWGQGTLVTVKP

SEQ ID NO: 193 - protein, 126 aa, synthetic construct
EVQLLESGGGEVQPGGSLRLSCAASGWAFGNYGMAWFRQAPGKEREFVSRLAWSGGSTDYVESVKGRFTISRDNAKNTLYLQMSSLRAEDTAVYYCARQRSYSRYDIRTPQTYDYWGQGTLVTVKP

SEQ ID NO: 194 - protein, 126 aa, synthetic construct
EVQLLESGGGEVQPGGSLRLSCAASGWAFSNYGMAWFRQAPGKEREFVSRLAWSGGSTDYVESVKGRFTISRDNAKNTLYLQMSSLRAEDTAVYYCARQRSYSRYDIRTPQTYDYWGQGTLVTVKP

SEQ ID NO: 195 - protein, 126 aa, synthetic construct
EVQLLESGGGEVQPGGSLRLSCAASGWAFGNYGMAWFRQAPGKEREFVSRLAWGGGSTDYVESVKGRFTISRDNAKNTLYLQMSSLRAEDTAVYYCARQRSYSGYDIRTPQTYDYWGQGTLVTVKP

SEQ ID NO: 196 - protein, 126 aa, synthetic construct
EVQLLESGGGEVQPGGSLRLSCAASGWAFGNYGMAWFRQAPGKEREFVSRLAWGGGSTDYVESVKGRFTISRDNAKNTLYLQMSSLRAEDTAVYYCARQRSYSRYGIRTPQTYDYWGQGTLVTVKP

SEQ ID NO: 197 - protein, 126 aa, synthetic construct
EVQLLESGGGEVQPGGSLRLSCAASGWAFGNYGMAWFRQAPGKEREFVSRLAWGGGSTDYVESVKGRFTISRDNAKNTLYLQMSSLRAEDTAVYYCARQRSYSGYGIRTPQTYDYWGQGTLVTVKP

SEQ ID NO: 198 - protein, 121 aa, synthetic construct
EVQLLESGGGEVQPGGSLRLSCAASGFSFSINAMGWYRQAPGKGLEWVAAIDSGRNTVYAESVKGRFTISRDNAKNTLYLQMSSLRAEDTAVYYCGLLKGNRVVSPSVAYWGQGTLVTVKP

SEQ ID NO: 199 - protein, 121 aa, synthetic construct
EVQLLESGGGEVQPGGSLRLSCAASGFSFSINAMGWYRQAPGKRREFVAAIESGRNTVYAESVKGRFTISRDNAKNTVYLQMSSLRAEDTAVYYCGLLKGNRVVSPSVAYWGQGTLVTVKP

SEQ ID NO: 200 - protein, 121 aa, synthetic construct
EVQLLESGGGEVQPGGSLRLSCAASGFSFSINAMGWYRQAPGKRREFVAAIYSGRNTVYAESVKGRFTISRDNAKNTVYLQMSSLRAEDTAVYYCGLLKGNRVVSPSVAYWGQGTLVTVKP

SEQ ID NO: 201 - protein, 121 aa, synthetic construct
EVQLLESGGGEVQPGGSLRLSCAASGFTFSINAMGWYRQAPGKRREFVAAIESGRNTVYAESVKGRFTISRDNAKNTVYLQMSSLRAEDTAVYYCGLLKGNRVVSPSVAYWGQGTLVTVKP

SEQ ID NO: 202 - protein, 121 aa, synthetic construct
EVQLLESGGGEVQPGGSLRLSCAASGFSFSINAMSWYRQAPGKRREFVAAIESGRNTVYAESVKGRFTISRDNAKNTVYLQMSSLRAEDTAVYYCGLLKGNRVVSPSVAYWGQGTLVTVKP

SEQ ID NO: 203 - protein, 121 aa, synthetic construct
EVQLLESGGGEVQPGGSLRLSCAASGFTFSSNAMGWYRQAPGKRREFVAAIESGRNTVYAESVKGRFTISRDNAKNTVYLQMSSLRAEDTAVYYCGLLKGNRVVSPSVAYWGQGTLVTVKP

SEQ ID NO: 204 - protein, 121 aa, synthetic construct
EVQLLESGGGEVQPGGSLRLSCAASGFSFSINAMGWYRQAPGKRREFVAAIESSRNTVYAESVKGRFTISRDNAKNTVYLQMSSLRAEDTAVYYCGLLKGNRVVSPSVAYWGQGTLVTVKP

SEQ ID NO: 205 - protein, 121 aa, synthetic construct
EVQLLESGGGEVQPGGSLRLSCAASGFSFSINAMGWYRQAPGKRREFVAAIESGSNTVYAESVKGRFTISRDNAKNTVYLQMSSLRAEDTAVYYCGLLKGNRVVSPSVAYWGQGTLVTVKP

SEQ ID NO: 206 - protein, 121 aa, synthetic construct
EVQLLESGGGEVQPGGSLRLSCAASGFSFSINAMGWYRQAPGKRREFVAAIESGRSTVYAESVKGRFTISRDNAKNTVYLQMSSLRAEDTAVYYCGLLKGNRVVSPSVAYWGQGTLVTVKP

SEQ ID NO: 207 - protein, 121 aa, synthetic construct
EVQLLESGGGEVQPGGSLRLSCAASGFSFSINAMGWYRQAPGKRREFVAAIESGRNTYYAESVKGRFTISRDNAKNTVYLQMSSLRAEDTAVYYCGLLKGNRVVSPSVAYWGQGTLVTVKP

SEQ ID NO: 208 - protein, 121 aa, synthetic construct
EVQLLESGGGEVQPGGSLRLSCAASGFSFSINAMGWYRQAPGKRREFVAAIYSGSSTVYAESVKGRFTISRDNAKNTVYLQMSSLRAEDTAVYYCGLLKGNRVVSPSVAYWGQGTLVTVKP

SEQ ID NO: 209 - protein, 121 aa, synthetic construct
EVQLLESGGGEVQPGGSLRLSCAVSGFSFSINAMGWYRQAPGKRREFVAAIESGRNTVYAESVKGRFTISRDNAKNTVYLQMSSLRAEDTAVYYCGLLKGNRVVSPSVAYWGQGTLVTVKP

SEQ ID NO: 210 - protein, 121 aa, synthetic construct
EVQLLESGGGEVQPGGSLRLSCAASGFSFSINAMGWYRQAPGKRREFVAAIESGRNTVYAVSVKGRFTISRDNAKNTVYLQMSSLRAEDTAVYYCGLLKGNRVVSPSVAYWGQGTLVTVKP

SEQ ID NO: 211 - protein, 121 aa, synthetic construct
EVQLLESGGGEVQPGGSLRLSCAASGFSFSINAMGWYRQAPGKGREFVAAIESGRNTVYAESVKGRFTISRDNAKNTVYLQMSSLRAEDTAVYYCGLLKGNRVVSPSVAYWGQGTLVTVKP

SEQ ID NO: 212 - protein, 121 aa, synthetic construct
EVQLLESGGGEVQPGGSLRLSCAASGFSFSINAMGWYRQAPGKRREFLAAIESGRNTVYAESVKGRFTISRDNAKNTVYLQMSSLRAEDTAVYYCGLLKGNRVVSPSVAYWGQGTLVTVKP

SEQ ID NO: 213 - protein, 122 aa, synthetic construct
QVQLEESGGGSVQAGETLRLSCTASGSTFGDSDMGWYRQAPGNACELVSIISSDGRTYYVDSVKGRFTISQDNAVSTVYLQMNSLKPEDTGVYYCAADLRQYCRDGRCCGYWGQGTQVTVSS

SEQ ID NO: 214 - protein, 122 aa, synthetic construct
QIQLVESGGGSVQAGETLRLSCTASGSTFGDSDMGWYRQAPGNACELVSIISSDGRTYYVDSVKGRFTISQDNAVSTVYLQMNSLNPEDTGVYYCAADLRLNCRDGRCCGYWGQGTQVTVSS

SEQ ID NO: 215 - protein, 123 aa, synthetic construct
QVHLMESGGGSVQAGETLRLSCTASGFTFANSDMGWYRQAPGNACELVSIISSHGGTTYYVDSVKGRFTISRHNAENTVYLRMTSLKPEDTALYYCVADPRSNCRGGYCCGYWGPGTQVTVSS

SEQ ID NO: 216 - protein, 124 aa, synthetic construct
EVQLVASGGGSVQAGETLRLSCTASGFTFADSAMGWYRKGPGNVCDLVAIIRTDGTTYYGDSAKGRFTISRDNAKSTLYLQMNSLKPEDTAVYFCAADRETSFIGGSWCVAKYWDQGTQVTVSS

SEQ ID NO: 217 - protein, 122 aa, synthetic construct
EVQLVESGGGSVQAGGSLRLSCTASRFTFDGPDMAWYRQAPGNACELVSIISADGRTYYTDSVKGRFTISRDNAKNTVFLYLNSLQPEDTAVYYCAPDPRRNCRGGYCCGNWGPGTQVTVSS

SEQ ID NO: 218 - protein, 122 aa, synthetic construct
QMQLVESGGGSVQAGETLRLSCTTSAFTFDGPDMAWYRQAPGNECVLVSIISADGRTYYADSVKGRFTISRDNAKNTVFLNLNSLQPEDTAVYYCALDPRKNCRGGYCCANWGPGTQVTVSS

SEQ ID NO: 219 - protein, 119 aa, synthetic construct
QVQLVESGGGSVQAGGSLRLSCTASGYIFCMGWFRQAPGKAREGIATIYTGGDSTYYDDSVKGRFTISRDNAKNTVYLQMNSLKPEDTAMYYCAAGGQECYLTNWVSYWGQGTQVTVSS

SEQ ID NO: 220 - protein, 123 aa, synthetic construct
QVKLVESGGGSVQAGGSLRLSCAASGYIYSSNCMGWFRQAPGKEREWVARIHTGSGSTYYADSVKGRFTISQDNAKNTVYLQMNSLRPEDTAMYDCAAGRVVLGAVVCTNEYWGQGTQVTVSS

SEQ ID NO: 221 - protein, 122 aa, synthetic construct
EVQLVESGGGLVQPGGSLRLSCTASGFTDDGPDMAWYRRAPGNECELVSIISADGRTYYTDSVKGRFTISRDNAKNTVFLYLNSLQPEDTAVYYCAPDPRRNCRGGDCCGNWGPGTQVTVSS

SEQ ID NO: 222 - protein, 121 aa, synthetic construct
QVQLVESGGGSVQAGETLRLSCTASGFTFDGPDMAWYRQAPGNECELVSIISADGRTYYTDSVKGRFTASQDNAKNTVSLYLKSLQPEDTAVYYCAADPRRNCRGNCCGNWGPGTQVTVSS

SEQ ID NO: 223 - protein, 122 aa, synthetic construct
EVQLVESGGGLVQPGGSLRLSCAASGSTFGDSDMGWYRQAPGKGCELVSIISSDGRTYYVDSVKGRFTISQDNSKNTLYLQMNSLRAEDTAVYYCAADLRQYCRDGRCCGYWGQGTLVTVSS

SEQ ID NO: 224 - protein, 122 aa, synthetic construct
EVQLVESGGGLVQPGGSLRLSCAASGSTFGDSDMGWYRQAPGKGCELVSIISSDGRTYYVDSVKGRFTISQDNSKNTVYLQMNSLRAEDTAVYYCAADLRQYCRDGRCCGYWGQGTLVTVSS

SEQ ID NO: 225 - protein, 122 aa, synthetic construct
EVQLVESGGGLVQPGGSLRLSCAASGSTFGDSDMGWYRQAPGKGCELVSIISSDGRTYYVDSVKGRFTISQDNAKNTLYLQMNSLRPEDTAVYYCAADLRQYCRDGRCCGYWGQGTLVTVSS

SEQ ID NO: 226 - protein, 122 aa, synthetic construct
EVQLVESGGGLVQPGGSLRLSCAASGSTFGDSDMGWYRQAPGKGCELVSIISSDGRTYYVDSVKGRFTISQDNAKNTVYLQMNSLRPEDTAVYYCAADLRQYCRDGRCCGYWGQGTLVTVSS

SEQ ID NO: 227 - protein, 122 aa, synthetic construct
EVQLVESGGGLVQPGGSLRLSCAASGSTFGDSDMGWYRQAPGKGCELVSIISSDGRTYYVDSVKGRFTISQDNSKNTLYLQMNSLRAEDTAVYYCAADLRLNCRDGRCCGYWGQGTLVTVSS

SEQ ID NO: 228 - protein, 122 aa, synthetic construct
EVQLVESGGGLVQPGGSLRLSCAASGSTFGDSDMGWYRQAPGKGCELVSIISSDGRTYYVDSVKGRFTISQDNSKNTVYLQMNSLRAEDTAVYYCAADLRLNCRDGRCCGYWGQGTLVTVSS

SEQ ID NO: 229 - protein, 122 aa, synthetic construct
EVQLVESGGGLVQPGGSLRLSCAASGSTFGDSDMGWYRQAPGKGCELVSIISSDGRTYYVDSVKGRFTISQDNAKNTLYLQMNSLRPEDTAVYYCAADLRLNCRDGRCCGYWGQGTLVTVSS

SEQ ID NO: 230 - protein, 122 aa, synthetic construct
EVQLVESGGGLVQPGGSLRLSCAASGSTFGDSDMGWYRQAPGKGCELVSIISSDGRTYYVDSVKGRFTISQDNAKNTVYLQMNSLRPEDTAVYYCAADLRLNCRDGRCCGYWGQGTLVTVSS

SEQ ID NO: 231 - protein, 123 aa, synthetic construct
EVQLLESGGGLVQPGGSLRLSCAASGFTFANSDMGWYRQAPGKGCELVSIISSHGGTTYYVDSVKGRFTISRDNSKNTLYLQMNSLRAEDTAVYYCVADPRSNCRGGYCCGYWGQGTLVTVSS

SEQ ID NO: 232 - protein, 123 aa, synthetic construct
EVQLLESGGGLVQPGGSLRLSCAASGFTFANSDMGWYRQAPGKGCELVSIISSHGGTTYYVDSVKGRFTISRDNAKNTLYLQMNSLRPEDTAVYYCVADPRSNCRGGYCCGYWGQGTLVTVSS

SEQ ID NO: 233 - protein, 124 aa, synthetic construct
QVQLVESGGGVVQPGRSLRLSCAASGFTFADSAMGWYRQAPGKGCELVAIIRTDGTTYYGDSAKGRFTISRDNSKNTLYLQMNSLRAEDTAVYFCAADRETSFIGGSWCVAKYWDQGTLVTVSS

SEQ ID NO: 234 - protein, 124 aa, synthetic construct
QVQLVESGGGVVQPGRSLRLSCAASGFTFADSAMGWYRQAPGKVCELVAIIRTDGTTYYGDSAKGRFTISRDNSKNTLYLQMNSLRAEDTAVYFCAADRETSFIGGSWCVAKYWDQGTLVTVSS

SEQ ID NO: 235 - protein, 124 aa, synthetic construct
QVQLVESGGGVVQPGRSLRLSCAASGFTFADSAMGWYRQAPGKGCELVAIIRTDGTTYYGDSAKGRFTISRDNAKNTLYLQMNSLRPEDTAVYFCAADRETSFIGGSWCVAKYWDQGTLVTVSS

SEQ ID NO: 236 - protein, 124 aa, synthetic construct
QVQLVESGGGVVQPGRSLRLSCAASGFTFADSAMGWYRQAPGKVCELVAIIRTDGTTYYGDSAKGRFTISRDNAKNTLYLQMNSLRPEDTAVYFCAADRETSFIGGSWCVAKYWDQGTLVTVSS

SEQ ID NO: 237 - protein, 122 aa, synthetic construct
EVQLVESGGGLVQPGGSLRLSCAASRFTFDGPDMAWYRQAPGKGCELVSIISADGRTYYTDSVKGRFTISRDNSKNTLYLQMNSLRAEDTAVYYCAPDPRRNCRGGYCCGNWGQGTTVTVSS

SEQ ID NO: 238 - protein, 122 aa, synthetic construct
EVQLVESGGGLVQPGGSLRLSCAASRFTFDGPDMAWYRQAPGKGCELVSIISADGRTYYTDSVKGRFTISRDNSKNTVYLQMNSLRAEDTAVYYCAPDPRRNCRGGYCCGNWGQGTTVTVSS

SEQ ID NO: 239 - protein, 122 aa, synthetic construct
EVQLVESGGGLVQPGGSLRLSCAASRFTFDGPDMAWYRQAPGKGCELVSIISADGRTYYTDSVKGRFTISRDNAKNTLYLQMNSLRPEDTAVYYCAPDPRRNCRGGYCCGNWGQGTTVTVSS

SEQ ID NO: 240 - protein, 122 aa, synthetic construct
EVQLVESGGGLVQPGGSLRLSCAASRFTFDGPDMAWYRQAPGKGCELVSIISADGRTYYTDSVKGRFTISRDNAKNTVYLQMNSLRPEDTAVYYCAPDPRRNCRGGYCCGNWGQGTTVTVSS

SEQ ID NO: 241 - protein, 122 aa, synthetic construct
EVQLVESGGGLVQPGGSLRLSCATSAFTFDGPDMAWYRQAPGKGCELVSIISADGRTYYADSVKGRFTISRDNSKNTVYLQMNSLRAEDTAVYYCALDPRKNCRGGYCCANWGQGTLVTVSS

SEQ ID NO: 242 - protein, 122 aa, synthetic construct
EVQLVESGGGLVQPGGSLRLSCATSAFTFDGPDMAWYRQAPGKGCELVSIISADGRTYYADSVKGRFTISRDNAKNTVYLQMNSLRPEDTAVYYCALDPRKNCRGGYCCANWGQGTLVTVSS

SEQ ID NO: 243 - protein, 119 aa, synthetic construct
QVQLVESGGGVVQPGGSLRLSCAASGYIFCMGWFRQAPGKGLEGIATIYTGGDSTYYDDSVKGRFTISRDNSKNTLYLQMNSLRAEDTAVYYCAAGGQECYLTNWVSYWGQGTLVTVSS

SEQ ID NO: 244 - protein, 119 aa, synthetic construct
QVQLVESGGGVVQPGGSLRLSCAASGYIFCMGWFRQAPGKGREGIATIYTGGDSTYYDDSVKGRFTISRDNAKNTLYLQMNSLRPEDTAVYYCAAGGQECYLTNWVSYWGQGTLVTVSS

SEQ ID NO: 245 - protein, 123 aa, synthetic construct
EVQLVESGGGLVQPGGSLRLSCAASGYIYSSNCMGWFRQAPGKGLEWVSRIHTGSGSTYYADSVKGRFTISRDNSKNTLYLQMNSLRAEDTAVYYCAAGRVVLGAVVCTNEYWGQGTLVTVSS

SEQ ID NO: 246 - protein, 123 aa, synthetic construct
EVQLVESGGGLVQPGGSLRLSCAASGYIYSSNCMGWFRQAPGKGLEWVSRIHTGSGSTYYADSVKGRFTISQDNSKNTLYLQMNSLRAEDTAVYYCAAGRVVLGAVVCTNEYWGQGTLVTVSS

SEQ ID NO: 247 - protein, 123 aa, synthetic construct
EVQLVESGGGLVQPGGSLRLSCAASGYIYSSNCMGWFRQAPGKGLEWVARIHTGSGSTYYADSVKGRFTISQDNSKNTLYLQMNSLRAEDTAVYYCAAGRVVLGAVVCTNEYWGQGTLVTVSS

SEQ ID NO: 248 - protein, 123 aa, synthetic construct
EVQLVESGGGLVQPGGSLRLSCAASGYIYSSNCMGWFRQAPGKGREWVSRIHTGSGSTYYADSVKGRFTISRDNAKNTLYLQMNSLRPEDTAVYYCAAGRVVLGAVVCTNEYWGQGTLVTVSS

SEQ ID NO: 249 - protein, 123 aa, synthetic construct
EVQLVESGGGLVQPGGSLRLSCAASGYIYSSNCMGWFRQAPGKGREWVSRIHTGSGSTYYADSVKGRFTISQDNAKNTLYLQMNSLRPEDTAVYYCAAGRVVLGAVVCTNEYWGQGTLVTVSS

SEQ ID NO: 250 - protein, 123 aa, synthetic construct
EVQLVESGGGLVQPGGSLRLSCAASGYIYSSNCMGWFRQAPGKGREWVARIHTGSGSTYYADSVKGRFTISQDNAKNTLYLQMNSLRPEDTAVYYCAAGRVVLGAVVCTNEYWGQGTLVTVSS

SEQ ID NO: 251 - protein, 122 aa, synthetic construct
EVQLVESGGGLVQPGGSLRLSCAASGFTDDGPDMAWYRQAPGKGCELVSIISADGRTYYTDSVKGRFTISRDNSKNTLYLQMNSLRAEDTAVYYCAPDPRRNCRGGDCCGNWGQGTTVTVSS

SEQ ID NO: 252 - protein, 122 aa, synthetic construct
EVQLVESGGGLVQPGGSLRLSCAASGFTDDGPDMAWYRQAPGKGCELVSIISADGRTYYTDSVKGRFTISRDNSKNTVYLQMNSLRAEDTAVYYCAPDPRRNCRGGDCCGNWGQGTTVTVSS

SEQ ID NO: 253 - protein, 122 aa, synthetic construct
EVQLVESGGGLVQPGGSLRLSCAASGFTDDGPDMAWYRQAPGKGCELVSIISADGRTYYTDSVKGRFTISRDNAKNTLYLQMNSLRPEDTAVYYCAPDPRRNCRGGDCCGNWGQGTTVTVSS

SEQ ID NO: 254 - protein, 122 aa, synthetic construct
EVQLVESGGGLVQPGGSLRLSCAASGFTDDGPDMAWYRQAPGKGCELVSIISADGRTYYTDSVKGRFTISRDNAKNTVYLQMNSLRPEDTAVYYCAPDPRRNCRGGDCCGNWGQGTTVTVSS

SEQ ID NO: 255 - protein, 121 aa, synthetic construct
EVQLVESGGGLVQPGGSLRLSCAASGFTFDGPDMAWYRQAPGKGCELVSIISADGRTYYTDSVKGRFTASQDNSKNTLYLQMNSLRAEDTAVYYCAADPRRNCRGNCCGNWGQGTLVTVSS

SEQ ID NO: 256 - protein, 121 aa, synthetic construct
EVQLVESGGGLVQPGGSLRLSCAASGFTFDGPDMAWYRQAPGKGCELVSIISADGRTYYTDSVKGRFTASQDNSKNTVYLQMNSLRAEDTAVYYCAADPRRNCRGNCCGNWGQGTLVTVSS

SEQ ID NO: 257 - protein, 121 aa, synthetic construct
EVQLVESGGGLVQPGGSLRLSCAASGFTFDGPDMAWYRQAPGKGCELVSIISADGRTYYTDSVKGRFTASQDNAKNTLYLQMNSLRPEDTAVYYCAADPRRNCRGNCCGNWGQGTLVTVSS

SEQ ID NO: 258 - protein, 121 aa, synthetic construct
EVQLVESGGGLVQPGGSLRLSCAASGFTFDGPDMAWYRQAPGKGCELVSIISADGRTYYTDSVKGRFTASQDNAKNTVYLQMNSLRPEDTAVYYCAADPRRNCRGNCCGNWGQGTLVTVSS

SEQ ID NO: 259 - protein, 115 aa, synthetic construct
QVQLVESGGGLVQPGGSLRLSCAASGFTFSTHAMSWYRQAPGKERELVAHISSTGGSTNYADSVKGRFTISRDNAKNTVYLQMNSLKPEDTAVYRCNARDWYEYWGRGTQVTVSS

SEQ ID NO: 260 - protein, 117 aa, synthetic construct
QVQLVESGGGLVQPGGSLRLSCVASGRLFDTYTMAWYRLPPGKQRELVADISRTGFTNYADSVKGRFTISRDNAKNTVYLQMNSLKPDDTAHYRCKVREPATMYEYWGQGTQVTVSS

SEQ ID NO: 261 - protein, 115 aa, synthetic construct
QVQLVESGGGSVQPGGSLRLSCAASGSIGSIHRWDWYRLCPGKQREWVATLNSEGGPTYADSVEGRFTISRDNAKNMVYLQMNSLKPEDTAVYRCSARTVPFEYWGRGTQVTVSS

SEQ ID NO: 262 - protein, 118 aa, synthetic construct
QVQLVESGGGLVQPGGSLRLSCAAAESMLSPYSMGWYRLPPGKQRELVASLGSGGRTTYADSVKGRFTISRDNAKNTAYLQMNSLKPEDTAVYRCNIRELLGRRYEYWSQGTQVTVSS

SEQ ID NO: 263 - protein, 119 aa, synthetic construct
QVQLVESGGGLVQPGGSLRLSCAASGFTFSSSHMTWVRQAPGKGLEWVSLINSSGRTAYVDSVAGRFTISRDNAKNTLYLQMTNLKPEDTAVYYCSKGGWDGLPSSSIRGQGTQVTVSS

SEQ ID NO: 264 - protein, 112 aa, synthetic construct
QVQLVESGGGLVPIGGSLRLSCAVSGLAFDRYTFNWYRQAPGKGREWVASIDSTSAVIDYEDTVKGRFTISRDNTKNTVYLQMNSLKPEDTAVYFCGRGGYYGQGTQVTVST

SEQ ID NO: 265 - protein, 117 aa, synthetic construct
QVQLVESGGGLVQPGGSLKLSCVASGFTDKPTYWSWYRQPPGKDRELVARMHTGGLGTAYPDSVAGRFTISTVNDKNTVYLQMNSLKPEDTAVYYCNAEISGGPDYWGQGTQVTVSS

SEQ ID NO: 266 - protein, 124 aa, synthetic construct
QVKLEESGGGLVQAGGSLRLSCAASGRTIDNYAMAWSRQAPGKDREFVATIDWGDGGARYANSVKGRFTISRDNAKGTMYLQMNNLEPEDTAVYSCAMARQSRVNLDVARYDYWGQGTQVTVSS

SEQ ID NO: 267 - protein, 124 aa, synthetic construct
EVQLVESGGGLVQPGGSLRLSCAASGRTIDNYAMAWVRQAPGKGLEWVSTIDWGDGGTRYANSVKGRFTISRDNSKNTLYLQMNSLRAEDTAVYYCAMARQSRVNLDVARYDYWGQGTLVTVSS

SEQ ID NO: 268 - protein, 124 aa, synthetic construct
QVQLVESGGGLVQPGGSLRLSCAASGRTIDNYAMAWVRQAPGKGLEWVATIDWGDGGTRYANSVKGRFTISRDNSKNTMYLQMNSLRAEDTAVYYCAMARQSRVNLDVARYDYWGQGTLVTVSS

SEQ ID NO: 269 - protein, 124 aa, synthetic construct
QVQLVESGGGLVQPGGSLRLSCAASGRTIDNYAMAWSRQAPGKGLEFVATIDWGDGGTRYANSVKGRFTISRDNSKNTMYLQMNSLRAEDTAVYYCAMARQSRVNLDVARYDYWGQGTLVTVSS

SEQ ID NO: 270 - protein, 124 aa, synthetic construct
QVQLVESGGGLVQPGGSLRLSCAASGRTIDNYAMAWSRQAPGKDREFVATIDWGDGGTRYANSVKGRFTISRDNSKGTMYLQMNSLRAEDTAVYSCAMARQSRVNLDVARYDYWGQGTLVTVSS

SEQ ID NO: 271 - protein, 124 aa, synthetic construct
EVQLVESGGGLVQPGGSLRLSCAASGRTIDNYAMAWSRQAPGKDREFVSTIDWGDGGTRYANSVKGRFTISRDNSKNTLYLQMNSLRAEDTAVYYCAMARQSRVNLDVARYDYWGQGTLVTVSS

SEQ ID NO: 272 - protein, 114 aa, synthetic construct
EVQLVESGGGLVQPGGSLRLSCAASGSIFTFNAMGWYRQAPGKQRELVAVIISGGSTHYVDSVKGRFTISRDNAKKMVYLQMNSLKPEDTAVYYCNVKKFGDYWGQGTQVTVSS

SEQ ID NO: 273 - protein, 116 aa, synthetic construct
DVQLVESGGGLVQAGGSLRLSCAASESISTINVMAWYRQAPGKQRELVAEITRSGRTNYVDSVKGRFTISRDNAKNTMYLQMNSLNLEDTAVYYCRTIDGSWREYWGQGTQVTVSS

SEQ ID NO: 274 - protein, 125 aa, synthetic construct
QVKLEESGGGLVQPGGSLRLSCVASGRTFSNYAMGWFRQAPGQEREFVAAINWNSRSTYYADSVKGRFTISRLNARNTVYLQMNRLKPEDTAVYDCAASHDSDYGGTNANLYDYWGQGTQVTVSS

SEQ ID NO: 275 - protein, 124 aa, synthetic construct
QVKLEESGGGLVQAGGSLRLSCVASGRTFSRTAMAWFRQAPGKEREFVATITWNSGTTRYADSVKGRFFISKDSAKNTIYLEMNSLEPEDTAVYYCAATAAAVITPTRGYYNYWGQGTQVTVSS

SEQ ID NO: 276 - protein, 116 aa, synthetic construct
EVQLVQSGAEVKKPGASVKVSCKTSGYTFTNYGINWVRQAPGQGLEWIGYIYIGAGDTDYSEKFKGRATITSDTSASTVYMELSSLRSEDTAVYYCAGTGTRFVYWGQGTLVTVSS

SEQ ID NO: 277 - protein, 116 aa, synthetic construct
EVQLVQSGAEVKKPGASVKVSCKTSGYTFTNYGINWVRQAPGQCLEWIGYIYIGAGDTDYSEKFKGRATITSDTSASTVYMELSSLRSEDTAVYYCAGTGTRFVYWGQGTLVTVSS

SEQ ID NO: 278 - protein, 113 aa, synthetic construct
VQLVQSGAEVKKPGASVKVSCKTSTFTNYGINWVRQAPGQGLEWIGYIYIGAGDTDYSEKFKGRATITSDTSASTVYMELSSLRSEDTAVYYCAGTGTRFVYWGQCTLVTVSS

SEQ ID NO: 279 - protein, 113 aa, synthetic construct
VQLVQSGAEVKKPGASVKVSCKTSTFTNYGINWVRQAPGQGLEWIGYIYIGAGDTDYSEKFKGRATITSDTSASTVYMELSSLRSEDTAVYYCAGTGTRFVYWGCGTLVTVSS

SEQ ID NO: 280 - protein, 116 aa, synthetic construct
EVQLVQSGAEVKKPGASVKVSCKTSGYTFTNYGINWVRQAPGQGCEWIGYIYIGAGDTDYSEKFKGRATITSDTSASTVYMELSSLRSEDTAVYYCAGTGTRFVYWGQGTLVTVSS

SEQ ID NO: 281 - protein, 113 aa, synthetic construct
VQLVQSGAEVKKPGASVKVSCKTSTFTNYGINWVRQAPGQGLEWIGYIYIGAGDTDYSEKFKGRATITSDTSASTVYMELSSLRSEDTAVYYCAGTGTRFVYCGQGTLVTVSS

SEQ ID NO: 282 - protein, 116 aa, synthetic construct
EVQLVQSGAEVKKPGASVKVSCKTSGYTFTNYGINWVRQAPGQGLEWIGYIYIGAGDTDYSEKFKGRATITSDTSASTVYMELSSLRSEDTAVYYCAGTGTCFVYWGQGTLVTVSS

SEQ ID NO: 283 - protein, 113 aa, synthetic construct
VQLVQSGAEVKKPGASVKVSCKTSTFTNYGINWVRQAPGQGLEWIGCIYIGAGDTDYSEKFKGRATITSDTSASTVYMELSSLRSEDTAVYYCAGTGTRFVYWGQGTLVTVSS

SEQ ID NO: 284 - protein, 13 aa, synthetic construct
WVRQAPGKCREWV

SEQ ID NO: 285 - protein, 13 aa, synthetic construct
WVRQAPGKECEWV

SEQ ID NO: 286 - protein, 11 aa, synthetic construct
RGCGTLVTVKP

SEQ ID NO: 287 - protein, 11 aa, synthetic construct
CGQGTLVTVKP

(19) United States
(12) Patent Application Publication
(10) Pub. No.: US 2026/0167735 A1
(43) Pub. Date: Jun. 18, 2026

(54) ENGINEERED SINGLE DOMAIN ANTIBODY CONSTRUCTS AND METHODS OF USE THEREOF

(71) Applicant: Regeneron Pharmaceuticals, Inc., Tarrytown, NY (US)

(72) Inventors: Vivian YEONG, Hillsdale, NJ (US); Yang SHEN, Scarsdale, NY (US); Chia-Yang LIN, Scarsdale, NY (US); Andrew J. MURPHY, Croton-on-Hudson, NY (US)

(73) Assignee: Regeneron Pharmaceuticals, Inc., Tarrytown, NY (US)

(21) Appl. No.: 19/420,477

(22) Filed: Dec. 15, 2025

Related U.S. Application Data
(60) Provisional application No. 63/734,412, filed on Dec. 16, 2024.

(51) Int. Cl. C07K 16/28

(52) U.S. Cl. CPC C07K 16/2878; C07K 16/2863; C07K 16/2866; C07K 16/2869; C07K 2317/524; C07K 2317/526; C07K 2317/53; C07K 2317/567; C07K 2317/569; C07K 2317/71; C07K 2317/75; C07K 2317/92

(57) ABSTRACT

The present disclosure relates to engineered antigen-binding molecules that include engineered single domain antibody variable domains having substituted cysteine residues. The engineered single domain antibody variable domains may form covalent or non-covalent inter-chain associations. Engineered antigen-binding molecules may include single domain antibody variable domains that target and are capable of agonizing a cell surface receptor and may be used, for example, in methods of agonizing certain receptors.

### 1. CROSS-REFERENCE TO RELATED APPLICATIONS

This application claims the priority benefit of U.S. provisional application No. 63/734,412, filed on Dec. 16, 2024, the contents of which are incorporated herein in their entirety by reference thereto.

### 2. SEQUENCE LISTING

The instant application contains a Sequence Listing which has been submitted electronically and is hereby incorporated by reference in its entirety. Said copy, created on Dec. 15, 2025 is named RGN-049US_SL.xml and is 2,871,213 bytes in size.

### 3. BACKGROUND

Functional antibodies and antibody-based constructs are an important therapeutic option for treatment of a wide variety of diseases. The binding of antigen-binding domains of some antibody constructs to a receptor may directly inhibit or activate the receptor, thus regulating cell signaling. Such antibody constructs may include those that employ multiple single domain antibodies (sdAbs) as antigen-binding domains to engage receptor subunits.

Antibody constructs that are agonists of multimeric cell membrane receptors exert their agonistic effect in some instances by bringing subunits of the receptor in close proximity to one another, for example when multiple antigen-binding domains of an antibody construct bind to receptor subunits. In many antibody constructs that have sdAb domains in place of Fab domains, the sdAb domains are flexibly connected to the antibody construct. This flexibility can allow receptor subunits bound to the two sdAb domains to remain farther apart than would be optimal to achieve the antibody construct's agonistic effect.

Thus, there exists a need in the art for novel approaches to promote associations between sdAb arms of antibody constructs, which may in some embodiments improve the agonistic activity of antibody constructs that target cell surface receptors.

### 4. SUMMARY

The present disclosure provides engineered antigen-binding molecules comprising sdAb domains with amino acid substitutions that cause the sdAbs to dimerize with one another, which may be via covalent or non-covalent associations.

Such engineered antigen-binding molecules can have decreased distance between sdAb domains relative to reference constructs without the amino acid substitutions. Without being bound by theory, the inventors believe that antigen-binding molecules that have the sdAb-sdAb dimerization have improved agonistic activity when compared to a parental antibody without the dimerization due to the capability of sdAb domains of the engineered antigen-binding molecules to bring bound receptor subunits closer to each other than receptor subunits bound by parental antigen-binding molecules. In addition to the distance between sdAb domains, the ability to achieve this improved agonistic activity may in some embodiments be affected by the binding geometry of the sdAb domains and target receptor subunits. Experimental results disclosed herein show that variant pairs of two different sdAb formats—VHH and sdVH—can effectively dimerize within an antigen-binding molecule construct, resulting in a desirable arrangement of binding arms which may in some embodiments enhance agonism or provide other advantageous properties. Exemplary engineered antigen-binding molecules are disclosed in Section 6.2.5 and numbered embodiments 1 to 120. Exemplary targeting moieties that can be included in engineered antigen-binding molecules of the disclosure are disclosed in Section 6.2.3.

The disclosure further provides nucleic acids encoding the engineered antigen-binding molecules of the disclosure and their components. The nucleic acids can be in the form of a single nucleic acid (e.g., a vector encoding all components of the engineered antigen-binding molecules) or a plurality of nucleic acids (e.g., two or more vectors encoding individual polypeptide chains). The disclosure further provides host cells and cell lines engineered to express the nucleic acids and the engineered antigen-binding molecules of the disclosure. The disclosure further provides methods of producing an engineered antigen-binding molecule of the disclosure. Exemplary nucleic acids, host cells, cell lines, and methods of producing engineered antigen-binding molecules are described in Section 6.3 and numbered embodiments 121 to 123 and 142 to 176.

The disclosure further provides pharmaceutical compositions comprising the engineered antigen-binding molecules of the disclosure. Exemplary pharmaceutical compositions are described in Section 6.4 and numbered embodiment 124.

Further provided herein are methods of using the engineered antigen-binding molecules, e.g., for agonizing a target molecule, activating an immune response, or treating cancer. Exemplary methods are described in Sections 6.5 and 6.6 and numbered embodiments 125 to 141.

### 5. BRIEF DESCRIPTION OF THE FIGURES

FIG. 1 is a cartoon illustration depicting a stapled single domain antibody (sdAb) of the disclosure comprising two polypeptides, each comprising, from N-terminus to C-terminus, a single domain antibody from variable heavy chain, such as a variable heavy domain of heavy chain (VHH) from camelid family or a single domain variable heavy chain (sdVH) from humans, an optional linker, a CH2 domain, and a CH3 domain. Each sdAb (e.g., VHH) comprises two amino acid substitutions which facilitate association of the two sdAbs (e.g., VHHs) into a “stapled” configuration, e.g., by formation of a disulfide bond or by noncovalent association.

FIGS. 2A-2E depict the result of an in silico screen for the positions to install disulfide bond between human VHs of a homodimer (PDB:7KQY). Sequence alignment (FIG. 2A) was performed for three solutions SS1 (FIG. 2B), SS3 (FIG. 2D) and SS4 (FIG. 2E) found by MOE software and one more solution, SS2 (FIG. 2B), based on previously reported disulfide bond for scFv stabilization. HCDR3s are outlined and Cysteine residues at the VH-VH dimer interface are shown as stick models in FIGS. 2B-2E. FIG. 2A discloses SEQ ID NOS: 276, 276, 277, 278, 277, 279, 280, 281, 282, and 283, respectively, in order of appearance.

FIGS. 3A-3D illustrate exemplary anti-OX40 sdAb structures. FIG. 3A shows the structure of a parental anti-OX40 sdAb that comprises unmodified VHH domains. FIG. 3B shows the structure of an exemplary anti-OX40 stapled sdAb, OX40-VHH SS2, which comprises Q108C and E44C substitutions in both VHH domains (numberings according to Kabat EU index) and an inter-chain disulfide bond between the VHH domains (represented by a line between the VHH domains). FIG. 3C shows the structure of an exemplary anti-OX40 stapled sdAb, OX40-VHH SS3, which comprises R106C and R45C substitutions in both VHH domains (numberings according to Kabat EU index) and an inter-chain noncovalent association between the VHH domains (represented by a line between the VHH domains). FIG. 3D shows the sequence alignment of sections of Framework 2 and Framework 4 between a reference human VH (PDB 7KQY), parental OX40 VHH, and anti-OX40 stapled sdAbs OX40-VHH SS2 and OX40-VHH SS3. Residues enclosed by boxes represent the Cys substitutions in the anti-OX40 stapled sdAbs. FIG. 3D discloses SEQ ID NOS: 41, 39, 284, 285, 42, 40, 286, and 287, respectively, in order of appearance.

FIGS. 4A-4B are graphs that show NFκB-Luc reporter activity associated with stapled sdAbs and control molecules. FIG. 4A shows the anchorage independent induction of NFκB-Luc reporter activity from Jurkat/NFκB-Luc/hOX40 in the presence of HEK293/hCD20 by stapled sdAbs and control molecules. FIG. 4B shows the anchorage dependent induction of NFκB-Luc reporter activity from Jurkat/NFκB-Luc/hOX40 in the presence of HEK293/anti-hFc by stapled sdAbs and control molecules.

FIGS. 5A-5C show nSEC-FLR-MS elution profiles of sdAbs following LysC digestion. FIG. 5A shows the nSEC-FLR-MS elution profile of the parental anti-OX40 sdAb. FIG. 5B shows the nSEC-FLR-MS elution profile of the anti-OX40 stapled sdAb, OX40-VHH SS2. FIG. 5C shows the nSEC-FLR-MS elution profile of the anti-OX40 stapled sdAb, OX40-VHH SS3.

FIGS. 6A-6H show in silico structures depicting VHH dimerization and the orientations of the two VHH domains of the control constructs and stapled sdAb constructs OX40-VHH SS2 and OX40-VHH SS3 derived from cryoEM imaging. FIG. 6A shows the structure of a conventional VH-VL pair. FIG. 6B shows inter-VHH (trans-disulfide) crosslinking of SS2 VHH domains. FIG. 6C shows intra-VHH (cis-disulfide) crosslinking and extension of FR4. FIG. 6D shows the alignment of the structures in FIGS. 6A-6C. FIG. 6E shows a conventional IgG antibody structure. FIG. 6F shows the structural model of a non-stapled VHH antibody. FIG. 6G shows the structural model of OX40-VHH SS2 and FIG. 6H shows the structure of OX40-VHH SS3.

FIGS. 7A-7F show nSEC-UV/MS elution profiles of unstapled, SS2-stapled, and SS3-stapled anti-Receptor A sdAb constructs before and after LysC digestion. FIG. 7A shows the nSEC-UV/MS elution profile of the undigested parental unstapled anti-Receptor A sdAb construct AF01. FIG. 7B shows the nSEC-UV/MS elution profile of the undigested SS2-stapled anti-Receptor A sdAb construct, AF15. FIG. 7C shows the nSEC-UV/MS elution profile of the undigested SS3-stapled anti-Receptor A sdAb construct, AF41. FIG. 7D shows the nSEC-UV/MS elution profile of the parental unstapled anti-Receptor A sdAb construct AF01 after LysC digestion. FIG. 7E shows the nSEC-UV/MS elution profile of the SS2-stapled anti-Receptor A sdAb construct, AF15, after LysC digestion. FIG. 7F shows the nSEC-UV/MS elution profile of the SS3-stapled anti-Receptor A sdAb construct, AF41, after LysC digestion.

FIGS. 8A-8H show nSEC-UV/MS elution profiles of four anti-Receptor A SS2-stapled sdVH constructs before and after LysC digestion. FIG. 8A shows the nSEC-UV/MS elution profile of the undigested anti-Receptor A sdAb construct, AF15. FIG. 8B shows the nSEC-UV/MS elution profile of the undigested anti-Receptor A sdAb construct, AF16. FIG. 8C shows the nSEC-UV/MS elution profile of the undigested anti-Receptor A sdAb construct, AF20. FIG. 8D shows the nSEC-UV/MS elution profile of the undigested anti-Receptor A sdAb construct, AF22. FIG. 8E shows the nSEC-UV/MS elution profile of the anti-Receptor A sdAb construct AF15 after LysC digestion. FIG. 8F shows the nSEC-UV/MS elution profile of the anti-Receptor A sdAb construct AF16 after LysC digestion. FIG. 8G shows the nSEC-UV/MS elution profile of the anti-Receptor A sdAb construct, AF20 after LysC digestion. FIG. 8H shows the nSEC-UV/MS elution profile of the anti-Receptor A sdAb construct AF22 after LysC digestion.

FIGS. 9A-9L show results of intact mass analysis of four anti-Receptor A SS2-stapled sdVH constructs before and after LysC digestion. FIG. 9A shows the intact mass analysis of the undigested anti-Receptor A sdAb construct, AF15. FIG. 9B shows the intact mass analysis of the undigested anti-Receptor A sdAb construct, AF16. FIG. 9C shows the intact mass analysis of the undigested anti-Receptor A sdAb construct, AF20. FIG. 9D shows the intact mass analysis of the undigested anti-Receptor A sdAb construct, AF22. FIG. 9E shows the intact mass analysis of the Fc portion of anti-Receptor A sdAb construct AF15. FIG. 9F shows the intact mass analysis of the Fc portion of anti-Receptor A sdAb construct AF16. FIG. 9G shows the intact mass analysis of the Fc portion of anti-Receptor A sdAb construct AF20. FIG. 9H shows the intact mass analysis of the Fc portion of anti-Receptor A sdAb construct AF22. FIG. 9I shows the intact mass analysis of the sdVH dimer portion of anti-Receptor A sdAb construct AF15. FIG. 9J shows the intact mass analysis of the sdVH dimer portion of anti-Receptor A sdAb construct AF16. FIG. 9K shows the intact mass analysis of the sdVH dimer portion of anti-Receptor A sdAb construct AF20. FIG. 9L shows the intact mass analysis of the sdVH dimer portion of anti-Receptor A sdAb construct AF22.

### 6. DETAILED DESCRIPTION

### 6.1. Definitions

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

Agonistic: The term “agonistic” as used herein with reference to a polypeptide, antibody, or binding molecule refers to the ability to increase signaling, activation, or activity of a protein to which the polypeptide, antibody, or binding molecule is bound.

And, or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelized antibodies, chimeric antibodies, bispecific or multispecific antibodies and anti-idiotypic (anti-id) antibodies. The antibodies can be of any isotype/class (e.g., IgG, IgE, IgM, IgD, IgA and IgY) or subclass (e.g., IgG1, IgG2, IgG3, IgG4, IgA1 and IgA2). Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding domain or amino-terminus of the antibody. The N-terminus is a variable region and at the C-terminus is a constant region; the CH3 and CL domains represent the carboxy-terminus of the heavy and light chain, respectively, of natural antibodies. For convenience, and unless the context dictates otherwise, the reference to an antibody also refers to antibody fragments as well as engineered antibodies that include non-naturally occurring antigen-binding domains and/or antigen-binding domains having non-native configurations.

Antigen-binding Domain: The term “antigen-binding domain” or “ABD” as used herein refers to the portion of a targeting moiety that is capable of specific, non-covalent, and reversible binding to a target molecule.

Associated: The term “associated” in the context of a binding molecule refers to a functional relationship between two or more polypeptide chains or portions of a polypeptide chain. In particular, the term “associated” means that two or more polypeptides are associated with one another, e.g., non-covalently through molecular interactions or covalently through one or more disulfide bridges or chemical cross-linkages, so as to produce a functional binding molecule. Examples of associations that might be present in a binding molecule of the disclosure include (but are not limited to) associations between homodimeric or heterodimeric Fc domains in an Fc region, associations between VH and VL regions in a Fab or scFv, associations between CH1 and CL in a Fab, and associations between CH3 and CH3 in a domain substituted Fab.

Bivalent: The term “bivalent” as used herein in the context of a binding molecule refers to a binding molecule that has two antigen-binding domains. The domains can be the same or different. Accordingly, a bivalent antigen-binding molecule can be monospecific or bispecific.

Cancer: The term “cancer” refers to a disease characterized by the uncontrolled (and often rapid) growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, adrenal gland cancer, autonomic ganglial cancer, biliary tract cancer, bone cancer, endometrial cancer, eye cancer, fallopian tube cancer, genital tract cancers, large intestinal cancer, cancer of the meninges, oesophageal cancer, peritoneal cancer, pituitary cancer, penile cancer, placental cancer, pleura cancer, salivary gland cancer, small intestinal cancer, stomach cancer, testicular cancer, thymus cancer, thyroid cancer, upper aerodigestive cancers, urinary tract cancer, vaginal cancer, vulva cancer, lymphoma, leukemia, lung cancer and the like.

Complementarity Determining Region or CDR: The terms “complementarity determining region” or “CDR,” as used herein, refer to the sequences of amino acids within antibody variable regions which confer antigen specificity and binding affinity. In general, there are three CDRs in each heavy chain variable region (CDR-H1, CDR-H2, CDR-H3) and three CDRs in each light chain variable region (CDR1-L1, CDR-L2, CDR-L3). Exemplary conventions that can be used to identify the boundaries of CDRs include, e.g., the Kabat definition, the Chothia definition, the ABD definition and the IMGT definition. See, e.g., Kabat, 1991, “Sequences of Proteins of Immunological Interest,” National Institutes of Health, Bethesda, Md. (Kabat numbering scheme); Al-Lazikani et al., 1997, J. Mol. Biol. 273:927-948 (Chothia numbering scheme); Martin et al., 1989, Proc. Natl. Acad. Sci. USA 86:9268-9272 (ABD numbering scheme); and Lefranc et al., 2003, Dev. Comp. Immunol. 27:55-77 (IMGT numbering scheme). For example, for classic formats, under Kabat, the CDR amino acid residues in the heavy chain variable domain (VH) are numbered 31-35 (CDR-H1), 50-65 (CDR-H2), and 95-102 (CDR-H3); and the CDR amino acid residues in the light chain variable domain (VL) are numbered 24-34 (CDR-L1), 50-56 (CDR-L2), and 89-97 (CDR-L3). Under Chothia, the CDR amino acids in the VH are numbered 26-32 (CDR-H1), 52-56 (CDR-H2), and 95-102 (CDR-H3); and the amino acid residues in VL are numbered 26-32 (CDR-L1), 50-52 (CDR-L2), and 91-96 (CDR-L3). By combining the CDR definitions of both Kabat and Chothia, the CDRs consist of amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT the CDR amino acid residues in the VH are numbered approximately 26-35 (CDR-H1), 51-57 (CDR-H2) and 93-102 (CDR-H3), and the CDR amino acid residues in the VL are numbered approximately 27-32 (CDR-L1), 50-52 (CDR-L2), and 89-97 (CDR-L3) (numbering according to “Kabat”). Under IMGT, the CDR regions of an antibody can be determined using the program IMGT/DomainGap Align. Public databases are available for identifying CDR sequences within an antibody.

Dimerization Moiety: The term “dimerization moiety” refers to a polypeptide chain or an amino acid sequence capable of facilitating an association between two polypeptide chains to form a dimer. A first dimerization moiety can associate with an identical second dimerization moiety, or can associate with a second dimerization moiety that is different from the first. In some embodiments, a dimerization moiety is an Fc domain, with the association of two Fc domains forming an Fc region. Thus, the Fc region can be homodimeric or heterodimeric.

EC50: The term “EC50” refers to the half maximal effective concentration of a molecule, such as an antigen-binding molecule, which induces a response halfway between the baseline and maximum after a specified exposure time. The EC50 essentially represents the concentration of a molecule where 50% of its maximal effect is observed. Thus, reduced or weaker binding is observed with an increased EC50, or half maximal effective concentration value.

Epitope: An epitope (or “antigenic determinant”) is a portion of an antigen (e.g., polypeptide antigen) recognized by an antibody or other antigen-binding moiety as described herein. An epitope can be linear or conformational.

Fab: The term “Fab” refers to a pair of polypeptide chains, the first comprising a variable heavy (VH) domain of an antibody N-terminal to a first constant domain (referred to herein as C1), and the second comprising variable light (VL) domain of an antibody N-terminal to a second constant domain (referred to herein as C2) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps that facilitate correct VH and VL pairings. For example, it is possible to replace the CH1 and CL domain pair in a Fab with a CH3-domain pair to facilitate correct modified Fab-chain pairing in heterodimeric molecules. It is also possible to reverse CH1 and CL, so that the CH1 is attached to VL and CL is attached to the VH, a configuration generally known as Crossmab (a type of “domain exchanged” arrangement). Alternatively, or in addition to, the use of substituted or swapped constant domains, correct chain pairing can be achieved by the use of universal light chains that can pair with both variable regions of a heterodimeric binding molecule of the disclosure. The term “Fab” encompasses single chain Fabs.

Fc Domain and Fc Region: The term “Fc domain” refers to a portion of the heavy chain that pairs with the corresponding portion of another heavy chain. The term “Fc region” refers to the region of antibody-based binding molecules formed by association of two heavy chain Fc domains. The two Fc domains within the Fc region may be the same or different from one another. In a native antibody the Fc domains are typically identical, but one or both Fc domains might advantageously be modified to allow for heterodimerization, e.g., via a knob-in-hole interaction and/or for purification, e.g., via star mutations.

Fv: The term “Fv” refers to the minimum antibody fragment derivable from an immunoglobulin that contains a complete target recognition and binding site. This region consists of a dimer of one heavy and one light chain variable domain in a tight, noncovalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target. The reference to a VH-VL dimer herein is not intended to convey any particular configuration. When present on a single polypeptide chain (e.g., a scFv), the VH and be N-terminal or C-terminal to the VL.

Host Cell or Recombinant Host Cell: The terms “host cell” and “recombinant host cell” as used herein refer to a cell that has been genetically engineered, e.g., through introduction of a heterologous nucleic acid. It should be understood that such terms are intended to refer not only to the particular subject cell but to the progeny of such a cell. Because certain modifications can occur in succeeding generations due to either mutation or environmental influences, such progeny may not, in fact, be identical to the parent cell, but are still included within the scope of the term “host cell” as used herein. A host cell can carry the heterologous nucleic acid transiently, e.g., on an extrachromosomal heterologous expression vector, or stably, e.g., through integration of the heterologous nucleic acid into the host cell genome. For purposes of expressing a multispecific binding molecule, a host cell can be a cell line of mammalian origin or mammalian-like characteristics, such as monkey kidney cells (COS, e.g., COS-1, COS-7), HEK293, baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells, or derivatives and/or engineered variants thereof. The engineered variants include, e.g., glycan profile modified and/or site-specific integration site derivatives.

Immune Response: The term “immune response” refers to an integrated bodily response to an antigen and preferably refers to a cellular immune response or a cellular as well as a humoral immune response. The immune response may be protective (also “preventive” or “prophylactic”) and/or therapeutic. The terms “inducing an immune response,” “eliciting an immune response,” and the like, as used herein, can indicate that there was no immune response against a particular antigen before administration of a particular composition (e.g., an MBM of the disclosure), but it may also indicate that there was a certain level of immune response against a particular antigen before such administration, and that after induction the immune response is enhanced. Thus, “inducing an immune response” also includes “enhancing an immune response”. Preferably, after inducing an immune response in a subject, said subject is protected from developing a disease (e.g., cancer) or the disease condition is ameliorated (e.g., tumor reduction, reduction in cancer cell number, etc.) by inducing an immune response. For example, an immune response against a tumor antigen may be induced in a patient having cancer or in a subject at risk of developing a cancer. Inducing an immune response in this case may mean that the disease condition of the subject is ameliorated, that the subject does not develop metastases, or that the subject at risk of developing cancer does not develop cancer.

Multivalent: The term “multivalent” as used herein refers to a binding molecule comprising two or more ABDs, on one, two or more polypeptide chains. A multivalent molecule may be monospecific, bispecific, or be specific for more than two epitopes, whether on the same antigen or different antigens.

Operably Linked: The term “operably linked” as used herein refers to a functional relationship between two or more regions of a polypeptide chain in which the two or more regions are linked so as to produce a functional polypeptide, or two or more nucleic acid sequences, e.g., to produce an in-frame fusion of two polypeptide components or to link a regulatory sequence to a coding sequence. In the context of a fusion protein or other polypeptide, the term “operably linked” means that two or more amino acid segments are linked so as to produce a functional polypeptide. In the context of a nucleic acid encoding a fusion protein, such as a multispecific binding molecule of the disclosure, “operably linked” means that the two nucleic acids are joined such that the amino acid sequences encoded by the two nucleic acids remain in-frame. In the context of transcriptional regulation, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system.

Polypeptide, Peptide and Protein: The terms “polypeptide”, “peptide” and “protein” are used interchangeably herein to refer to a polymer of amino acid residues.

Single Chain Fab or scFab: The term “single chain Fab” or “scFab” as used herein refers an ABD comprising a VH domain, a CH1 domain, a VL domain, a CL domain and a linker. In some embodiments, the foregoing domains and linker are arranged in one of the following orders in a N-terminal to C-terminal orientation: (a) VH-CH1-linker-VL-CL, (b) VL-CL-linker-VH-CH1, (c) VH-CL-linker-VL-CH1 or (d) VL-CH1-linker-VH-CL. Linkers are suitably noncleavable linkers of at least 30 amino acids, preferably between 32 and 50 amino acids. Single chain Fab fragments are typically stabilized via the natural disulfide bond between the CL domain and the CH1 domain. In addition, these single chain Fab molecules might be further stabilized by generation of interchain disulfide bonds via insertion of cysteine residues (e.g., at position 44 in the VH domain and position 100 in the VL domain according to Kabat numbering).

Single Chain Fv or scFv: The term “single chain Fv” or “scFv” as used herein refers to a polypeptide chain comprising the VH and VL domains of antibody, where these domains are present in a single polypeptide chain. Preferably, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds. (1994), Springer-Verlag, New York, pp. 269-315.

Single Domain Antibody or sdAb: The term “single domain antibody” or “sdAb” as used herein refers to an antibody or antigen binding fragment thereof comprising a single binding domain (e.g., heavy chain variable region) capable of binding a target molecule without pairing with a corresponding CDR-containing polypeptide (e.g., a light chain). An sdAb or sdAb fragment can be derived from a VH, a VHH, or from a non-antibody scaffold protein, for example a designed ankyrin repeat protein (darpin), an avimer, an anticalin/lipocalin, a centyrin or a fynomer. A sdAb typically lacks a CH1 domain and thus cannot associate with a light chain.

Single Domain VH Antibody or sdVH: The term “single domain VH” or “sdVH” as used herein refers to a variable region of an sdAb that is not of camelid or cartilaginous fish origin. An sdVH can be, for example, of human or non-human mammalian origin. A basic sdVH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

Specifically (or Selectively) Binds: The term “specifically (or selectively) binds” to an antigen or an epitope refers to a binding reaction that is determinative of the presence of a cognate antigen or an epitope in a heterogeneous population of proteins and other molecules. The binding reaction can be but need not be mediated by an antibody or antibody fragment. The term “specifically binds” does not exclude cross-species reactivity. For example, an antigen-binding domain (e.g., an antigen-binding fragment of an antibody) that “specifically binds” to an antigen from one species may also “specifically bind” to that antigen in one or more other species. Thus, such cross-species reactivity does not itself alter the classification of an antigen-binding domain as a “specific” binder. In certain embodiments, an antigen-binding domain of the disclosure that specifically binds to a human antigen has cross-species reactivity with one or more non-human mammalian species, e.g., a primate species (including but not limited to one or more of Macaca fascicularis, Macaca mulatta, and Macaca nemestrina) or a rodent species, e.g., Mus musculus.

Subject: The term “subject” includes human and non-human animals. Non-human animals include all vertebrates, e.g., mammals and non-mammals, such as non-human primates, sheep, dog, cow, chickens, amphibians, and reptiles. In certain embodiments, the subject is human. Except when noted, the terms “patient” or “subject” are used herein interchangeably.

T Cell Antigen: The term “T cell antigen” (also “TCA”) as used herein refers to any biological molecule (e.g., protein, carbohydrate, lipid or combination thereof) that is present on and/or expressed by a T cell. In some embodiments, at least a portion of a T cell antigen is extracellular (e.g., is a cell surface protein or transmembrane protein having at least one extracellular domain). Particular T cell antigens contemplated herein include, but are not limited to, CD3 and CD28. In some embodiments, the T cell antigen is CD3.

Targeting Moiety: The term “targeting moiety” as used herein refers to any molecule or binding portion thereof that can specifically bind to an antigen. Exemplary targeting moieties include, but are not limited to, antibodies and antigen binding portions thereof (e.g., Fab, scFv, sdAb, etc.). A targeting moiety may be described with reference to the antigen to which it specifically binds. Thus, for example, a “T cell antigen targeting moiety” (or “TCA targeting moiety”) refers to a molecule or binding portion thereof that can specifically bind to a T cell antigen. The TCA targeting moiety can also have a functional activity in addition to binding a T cell antigen. For example, a TCA targeting moiety that is a CD3 targeting moiety (e.g., an anti-CD3 antibody or an antigen binding portion thereof) may facilitate clustering and activation of CD3 on a surface of a T cell, while a TCA targeting moiety that is a CD28 targeting moiety (e.g., an anti-CD28 antibody or an antigen binding portion thereof) may activate CD28 signaling in a T cell. Similarly a “TCR targeting moiety” refers to a molecule or binding portion thereof that can specifically bind to a T cell receptor.

Tetravalent: The term “tetravalent” as used herein refers to a binding molecule that has four antigen-binding domains. In certain embodiments, all four of the antigen-binding domains bind to the same epitope. In some embodiments, three of the antigen-binding domains bind to the same epitope and the other antigen-binding domain binds to a different epitope, whether of the same target molecule or different target molecules. In other embodiments, two of the antigen-binding domains bind to the same epitope and the other two antigen-binding domains bind to a different epitope, whether of the same target molecule or different target molecules. In further embodiments, all four of the antigen-binding domains bind to different epitopes, whether on the same target molecule or on any combination of two or more different target molecules. Accordingly, a tetravalent binding molecule may be monospecific, bispecific, trispecific, or tetraspecific.

Tumor: The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

Tumor-Associated Antigen: The term “tumor-associated antigen” (also “tumor associated antigen”) or “TAA” refers to a molecule (typically a protein, carbohydrate, lipid or some combination thereof) that is expressed on the surface of a cancer cell, either entirely or as a fragment (e.g., as a peptide presented by an MHC molecule), and which is useful for the preferential targeting of a pharmacological agent to the cancer cell. In some embodiments, a TAA is a marker expressed by both normal cells and cancer cells, e.g., a lineage marker, e.g., CD19 on B cells. In some embodiments, a TAA is a cell surface molecule that is overexpressed in a cancer cell in comparison to a normal cell, for instance, 1-fold over expression, 2-fold overexpression, 3-fold overexpression or more in comparison to a normal cell. In some embodiments, a TAA is a cell surface molecule that is inappropriately synthesized in the cancer cell, for instance, a molecule that contains deletions, additions or mutations in comparison to the molecule expressed on a normal cell. In some embodiments, a TAA will be expressed exclusively on the cell surface of a cancer cell, entirely or as a fragment (e.g., as a peptide presented by an MHC molecule), and not synthesized or expressed on the surface of a normal cell. Accordingly, the term “TAA” encompasses antigens that are specific to cancer cells, sometimes known in the art as tumor-specific antigens (“TSAs”).

Universal Light Chain, ULC: The term “universal light chain” or “ULC” as used herein in the context of an antigen-biding domain refers to a light chain polypeptide capable of pairing with the heavy chain region of the antigen-biding domain and also capable of pairing with other heavy chain regions. Universal light chains are also known as “common light chains.”

VHH: The term “VHH” refers to a variable region of an sdAb of camelid or cartilaginous fish origin. A VHH variable region can bind to a target molecule in the absence of a light chain. A basic VHH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

VH: The term “VH” refers to the variable region of an immunoglobulin heavy chain of an antibody, including the heavy chain of an Fv, scFv, dsFv or Fab.

VL: The term “VL” refers to the variable region of an immunoglobulin light chain, including the light chain of an Fv, scFv, dsFv or Fab.

### 6.2. Engineered Antigen-Binding Molecules

The present disclosure provides engineered antigen-binding molecules that include two sdAb variable domains (which may be referred to herein as a first sdAb variable domain and a second sdAb variable domain) that include one or more engineered cysteine residues. Such antigen-binding molecules may comprise, for example, two polypeptide chains, each one of which comprises a sdAb variable domain. In some embodiments, an engineered cysteine residue in a first sdAb variable domain forms an inter-chain covalent bond (e.g., via a disulfide linkage) with an engineered cysteine residue in a second sdAb variable domain. In some embodiments, a pair of engineered cysteine residues in a first sdAb variable domain forms an intra-chain covalent bond within the first sdAb variable domain, and a pair of engineered cysteine residues in a second sdAb variable domain also forms an intra-chain covalent bond within the second sdAb variable domain. Without wishing to be bound by theory, the inventors believe that such intra-chain covalent bonds between engineered cysteine residues may promote association of the first and second sdAb variable domains with each other, thereby shortening the distance between the first and second sdAb variable domains within the antigen-binding molecule. This promotion of association could result from a conformational change in the sdAb variable domains resulting from the intra-chain covalent bond that makes a portion of each sdAb variable domain associate non-covalently with the other sdAb variable domain.

The inter-chain associations resulting from the presence of engineered cysteine residues in the sdAb variable domains may reduce the distance between sdAbs in antigen-binding constructs of the disclosure as compared to a corresponding antigen-binding construct lacking the engineered cysteine residues. Incorporating engineered cysteine residues as described herein may also improve the activity of an engineered antigen-binding molecule as compared to a corresponding antigen-binding molecule lacking the engineered cysteine residues, e.g., having native framework region sequences. Antigen-binding molecules capable of stimulating signaling through a cell membrane receptor may become capable of stimulating signaling to a greater degree by including engineered cysteine residues as described herein.

6.2.1. Engineered sdAb Variable Regions

Embodiments of engineered antigen-binding molecules of the disclosure may comprise two polypeptide chains, each of which comprises an sdAb that comprises one or more engineered cysteine residues (e.g., a cysteine residue that is a substitution for a non-cysteine residue in a reference sequence, such as a naturally-occurring framework region sequence). In some embodiments, the sdAbs associate with one another more strongly than in a reference antigen-binding molecule that lacks the engineered cysteine residues but is otherwise identical. Such association may be due to an inter-chain disulfide bond between engineered cysteine residues in the sdAb variable domains. Such association may also be due to a non-covalent association of a portion of one sdAb variable domain with the other, and vice-versa. A strengthened non-covalent association may be evidenced by a greater affinity (e.g., a lower binding constant Kd) between the two sdAbs than between a reference pair of sdAbs that lack the engineered cysteine residues but are otherwise identical to the two sdAbs. Without wishing to be bound by theory, the inventors believe that an intra-chain disulfide bond between engineered cysteine residues in each of the sdAb variable domains may result in a conformational change in the sdAb variable domains that increases the affinity of the sdAb variable domains for each other. This may involve the FR4 region of each sdAb variable domain associating with the other sdAb variable domain, which may be referred to as FR4 domain swapping and may exhibit conformational fluidity. Embodiments in which sdAb variable domains form an association as described herein, whether covalent or non-covalent, are referred to herein in some instances as being “stapled.”

In some embodiments, a pair of sdAb variable domains form an inter-chain association in the engineered antigen-binding molecule such that at least a portion of sdAb variable domains elute as dimers in a size-exclusion chromatography (SEC) column after sdAb variable domains are proteolytically cleaved from an antigen-binding molecule construct. For example, an engineered antigen-binding molecule may comprise two polypeptide chains, each of which includes a dimerization moiety (e.g., an Fc domain) and an sdAb variable domain separated by a protease cleavage site, wherein the dimerization moieties of the two polypeptide chains associate with one another to form a bivalent antigen-binding molecule. In versions of such constructs in which the sdAb variable domains are not “stapled,” protease cleavage followed by SEC would result in a peak corresponding to the size of dimerized dimerization moieties and a peak corresponding to the size of sdAb variable domain monomers, but would not result in a peak corresponding to the size of sdAb variable domain dimers. In contrast, in versions of antigen-binding molecule constructs in which the sdAb variable domains are stapled (e.g., due to covalent or non-covalent associations resulting from the presence of the engineered cysteine residues), protease cleavage followed by SEC would result in a peak corresponding to the size of dimerized dimerization moieties and a peak corresponding to the size of dimerized sdAb variable domains. Other peaks could also be present in such SEC assays. For example, in embodiments in which sdAb variable domains are stapled via a non-covalent association, a peak corresponding to the size of sdAb variable domain monomers could be present in addition to a peak corresponding to the size of sdAb variable domain dimers. In some embodiments, the ratio of dimerized sdAb variable domains to monomeric sdAb variable domains is at least 0.1:1, 0.25:1, 0.5:1, 0.75:1, 1:1, 2:1, 3:1, 4:1, 5:1, 6:1, 7:1, 8:1, 9:1, or 10:1. In some embodiments, the Kd between two sdAbs in an engineered antigen-binding molecule of the disclosure is less than 1×10⁻³ M, 1×10⁻⁴ M, 1×10⁻⁵ M, 1×10⁻⁶ M, 1×10⁻⁷ M, 1×10⁻⁸ M, or 1×10⁻⁹ M.

The locations of engineered cysteine residues in the sdAb variable domains may be chosen in order to effectuate an association between the two sdAb variable domains, such as via a disulfide bond between engineered cysteine residues. This can be accomplished by, for example, examining a structural model of the sdAbs in an engineered antigen-binding molecule and identifying residues in a position and orientation that would result in cysteine residues substituted in those positions being in close enough proximity to form an inter-chain disulfide bond. As another example, residues to be substituted with cysteine residues can be chosen based on position and proximity such that cysteine residues substituted in those positions would form an intra-chain disulfide bond. In some embodiments, an intra-chain sulfide bond is formed between an engineered cysteine residue in FR2 and an engineered cysteine residue in FR4 of a first sdAb variable domain. In some embodiments, the intra-chain disulfide bond results in the FR4 region adopting an extended orientation such that it can associate with a second sdAb in the engineered antigen-binding molecule having the same engineered cysteine residues and disulfide bond. In some embodiments, an inter-chain disulfide bond is formed between an engineered cysteine residue in FR2 of a first sdAb and an engineered cysteine residue in FR4 of a second sdAb.

In some embodiments, an engineered cysteine residue is a cysteine residue that is a substitution of cysteine for a non-cysteine residue in a reference sequence. A reference sequence may be, for example, a framework region (e.g., FR1, FR2, FR3, FR4) sequence present in a naturally-occurring VHH or VH (e.g., an sdVH). Such framework region sequences may be identified according to methods known in the art, such as the Kabat, Chothia, or IMGT numbering schemes. In some embodiments, the reference sequence is a native VHH FR2 sequence WVRQAPGKEREWV (SEQ ID NO:39) or a native VHH FR4 sequence RGQGTLVTVKP (SEQ ID NO:40). In some embodiments, the reference sequence is a native VH FR2 sequence WVRQAPGQGLEWI (SEQ ID NO:41) or a native VH FR4 sequence WGQGTLVTVSS (SEQ ID NO:42).

In some embodiments, an engineered cysteine residue is a cysteine residue that is not present in any of SEQ ID NOs: 39-42.

In some embodiments, an sdAb in an engineered antigen-binding molecule of the disclosure comprises a cysteine at a position corresponding to one or more of the following positions of a human VH, numbering according to Kabat: G44, L45, W106, or Q108. In some embodiments, the sdAb comprises a cysteine at a position corresponding to position G44 of a human VH, numbering according to Kabat. In some embodiments, the sdAb comprises a cysteine at a position corresponding to position L45 of a human VH, numbering according to Kabat. In some embodiments, the sdAb comprises a cysteine at a position corresponding to position W106 of a human VH, numbering according to Kabat. In some embodiments, the sdAb comprises a cysteine at a position corresponding to position Q108 of a human VH, numbering according to Kabat. In some embodiments, the sdAb comprises a cysteine at a position corresponding to position G44 of a human VH and a position corresponding to position Q108 of a human VH, numbering according to Kabat. In some embodiments, the sdAb comprises a cysteine at a position corresponding to position L45 of a human VH and a position corresponding to position W106 of a human VH, numbering according to Kabat. As used herein, a position corresponding to a given position of a human VH is identified using sequence alignment tools known in the art. A particularly preferred sequence alignment tool is EMBOSS Needle Pairwise Sequence Alignment software tool based on the Needleman and Wunsch algorithm (Needleman & Wunsch, 1970, J. Mol. Biol. 48(3):443-53) (available on the World Wide Web at ebi.ac.uk/Tools/psa/emboss_needle/). An example of a position corresponding to position G44 of a human VH is position E44 of a camelid VHH. An example of a position corresponding to position L45 of a human VH is position R45 of a camelid VHH. An example of a position corresponding to position W106 of a human VH is position R106 of a camelid VHH. An example of a position corresponding to position Q108 of a human VH is position Q108 of a camelid VHH. It will be understood by those skilled in the art that other corresponding positions may be determined for other heavy chain-based sdAbs in a similar fashion.

In some embodiments, an sdAb in an engineered antigen-binding molecule of the disclosure is a VHH and comprises a cysteine at one or more of the following positions, numbering according to Kabat: 44, 45, 106, or 108. In some embodiments, the VHH comprises a cysteine at position 44, numbering according to Kabat. In some embodiments, the VHH comprises a cysteine at position 45, numbering according to Kabat. In some embodiments, the VHH comprises a cysteine at position 106, numbering according to Kabat. In some embodiments, the VHH comprises a cysteine at position 108, numbering according to Kabat. In some embodiments, the VHH comprises a cysteine at position 44 and position 108, numbering according to Kabat. In some embodiments, a cysteine at position 44 of a first VHH in an engineered antigen-binding molecule forms an inter-chain disulfide bond with a cysteine at position 108 of a second VHH, numbering according to Kabat. In some embodiments, the VHH comprises a cysteine at position 45 and position 106, numbering according to Kabat. In some embodiments, a cysteine at position 45 of a first VHH of an engineered antigen-binding molecule forms an intra-chain disulfide bond with a cysteine at position 106 of the first VHH, and a cysteine at position 45 of a second VHH forms an intra-chain disulfide bond with a cysteine at position 106 of the second VHH, numbering according to Kabat. In some embodiments, the cysteine at position 44 is an E44C substitution, numbering according to Kabat. In some embodiments, the cysteine at position 45 is an R45C substitution, numbering according to Kabat. In some embodiments, the cysteine at position 106 is an R106C substitution, numbering according to Kabat. In some embodiments, the cysteine at position 108 is a Q108C substitution, numbering according to Kabat.

In some embodiments, an sdAb in an engineered antigen-binding molecule of the disclosure is sdVH and comprises a cysteine at one or more of the following positions, numbering according to Kabat: 44, 45, 106, or 108. In some embodiments, the sdVH comprises a cysteine at position 44, numbering according to Kabat. In some embodiments, the sdVH comprises a cysteine at position 45, numbering according to Kabat. In some embodiments, the sdVH comprises a cysteine at position 106, numbering according to Kabat. In some embodiments, the sdVH comprises a cysteine at position 108, numbering according to Kabat. In some embodiments, the sdVH comprises a cysteine at position 44 and position 108, numbering according to Kabat. In some embodiments, a cysteine at position 44 of a first sdVH in an engineered antigen-binding molecule forms an inter-chain disulfide bond with a cysteine at position 108 of a second sdVH, numbering according to Kabat. In some embodiments, the sdVH comprises a cysteine at position 45 and position 106, numbering according to Kabat. In some embodiments, a cysteine at position 45 of a first sdVH of an engineered antigen-binding molecule forms an intra-chain disulfide bond with a cysteine at position 106 of the first sdVH, and a cysteine at position 45 of a second sdVH forms an intra-chain disulfide bond with a cysteine at position 106 of the second sdVH, numbering according to Kabat. In some embodiments, the cysteine at position 44 is an E44C substitution, numbering according to Kabat. In some embodiments, the cysteine at position 44 is a G44C substitution, numbering according to Kabat. In some embodiments, the cysteine at position 45 is an L45C substitution, numbering according to Kabat. In some embodiments, the cysteine at position 106 is a W106C substitution, numbering according to Kabat. In some embodiments, the cysteine at position 108 is a Q108C substitution, numbering according to Kabat.

In some embodiments a first sdAb variable domain and a second sdAb variable domain of an engineered antigen-binding molecule of the invention have identical amino acid sequences. In some embodiments, both the first sdAb variable domain and the second sdAb variable domain have cysteine residues at positions corresponding to position G44 and Q108 of a human VH, numbering according to Kabat. In some embodiments, the cysteine at a position corresponding to human VH position G44 of a first sdAb variable domain forms a disulfide bond with the cysteine at a position corresponding to human VH position Q108 of a second sdAb, numbering according to Kabat. In some embodiments in which the first sdAb variable domain and the second sdAb variable domain are VHHs, both the first VHH and the second VHH have cysteine residues at positions 44 and 108, numbering according to Kabat. In some embodiments, the cysteine residue at position 44 of a first VHH forms a disulfide bond with the cysteine residue at position 108 of the second VHH. In some embodiments in which the first sdAb variable domain and the second sdAb variable domain are sdVHs, both the first sdVHs and the second sdVHs have cysteine residues at positions 44 and 108, numbering according to Kabat. In some embodiments, the cysteine residue at position 44 of a first sdVH forms a disulfide bond with the cysteine residue at position 108 of the second sdVH. In some embodiments, both the first sdAb variable domain and the second sdAb variable domain have cysteine residues at positions corresponding to L45 and W106 of a human VH, numbering according to Kabat. In some embodiments in which the first sdAb variable domain and the second sdAb variable domain are VHHs, both the first VHH and the second VHH have cysteine residues at positions 45 and 106, numbering according to Kabat. In some embodiments the cysteine residue at position 45 of the first VHH forms a disulfide bond with the cysteine residue at position 106 of the first VHH, and the cysteine residue at position 45 of the second VHH forms a disulfide bond with the cysteine residue at position 106 of the second VHH. In some embodiments in which the first sdAb variable domain and the second sdAb variable domain are sdVHs, both the first sdVH and the second sdVH have cysteine residues at positions 45 and 106, numbering according to Kabat. In some embodiments the cysteine residue at position 45 of the first sdVH forms a disulfide bond with the cysteine residue at position 106 of the first sdVH, and the cysteine residue at position 45 of the second sdVH forms a disulfide bond with the cysteine residue at position 106 of the second sdVH.

In some embodiments, a first sdAb variable domain and a second sdAb variable domain of an engineered antigen-binding molecule do not have identical amino acid sequences. In some embodiments, a first sdAb variable domain has a cysteine residue at a position corresponding to position G44 of a human VH and does not have a cysteine residue at a position corresponding to position Q108 of a human VH, and a second sdAb variable domain has cysteine residue at a position corresponding to position Q108 of a human VH and does not have a cysteine residue at a position corresponding to position G44 of a human VH, numbering according to Kabat. In some embodiments in which the first sdAb variable domain and the second sdAb variable domain are VHHs a first VHH has a cysteine residue at position 44 and does not have a cysteine residue at position 108, and a second VHH has cysteine residue at position 108 and does not have a cysteine residue at position 44 of a human VH, numbering according to Kabat. In some embodiments, the cysteine residue at position 44 of the first VHH forms a disulfide bond with the cysteine residue at position 108 of the second VHH. In some embodiments in which the first sdAb variable domain and the second sdAb variable domain are sdVHs a first sdVH has a cysteine residue at position 44 and does not have a cysteine residue at position 108, and a second sdVH has cysteine residue at position 108 and does not have a cysteine residue at position 44 of a human VH, numbering according to Kabat. In some embodiments, the cysteine residue at position 44 of the first sdVH forms a disulfide bond with the cysteine residue at position 108 of the second sdVH.

In some embodiments, an engineered antigen-binding molecule comprises a first polypeptide chain and a second polypeptide chain, wherein the first polypeptide chain comprises a first sdAb variable domain (e.g., a first VHH or a first sdVH) and a first dimerization moiety and the second polypeptide chain comprises a second sdAb variable domain (e.g., a second VHH or a second sdVH) and a second dimerization moiety. In some embodiments, the engineered antigen-binding molecule comprises an inter-chain disulfide bond between the first sdAb variable domain and the second sdAb variable domain and does not comprise any additional inter-chain disulfide bonds. In some embodiments, the first dimerization moiety and the second dimerization moiety are Fc domains comprising IgG hinge regions, and the engineered antigen-binding molecule comprises an inter-chain disulfide bond between the first sdAb variable domain and the second sdAb variable domain and one or more inter-chain disulfide bonds between the hinge regions.

In some embodiments, an engineered antigen-binding molecule comprises a first polypeptide chain and a second polypeptide chain, wherein the first polypeptide chain comprises a first sdAb variable domain (e.g., a first VHH or a first sdVH) and a first dimerization moiety and the second polypeptide chain comprises a second sdAb variable domain (e.g., a second VHH or a second sdVH) and a second dimerization moiety. In some embodiments, the first dimerization moiety and the second dimerization moiety do not comprise Fab domains, CH1 domains, or light chain domains.

### 6.2.2. IgG Constant Domains

Embodiments of engineered antigen-binding molecules disclosed herein may include IgG constant domains, such as, for example, IgG Fc domains. In some embodiments, an engineered antigen-binding molecule comprises a first polypeptide chain and a second polypeptide chain, wherein the first polypeptide chain comprises a first sdAb variable domain (e.g., a first VHH or a first sdVH) and a first dimerization moiety and the second polypeptide chain comprises a second sdAb variable domain (e.g., a second VHH or a second sdVH) and a second dimerization moiety. A dimerization moiety describes any polypeptide chain or amino acid sequence capable of facilitating an association between two polypeptide chains to form a dimer. In some embodiments, the first and/or second dimerization moiety is an Fc domain.

In native antibodies, the heavy chain Fc domain of IgG is composed of two heavy chain constant domains (CH2 and CH3). These dimerize to create an Fc region. The Fc domains that can be incorporated into engineered antigen-binding molecules of the present disclosure can be derived from any IgG subclass (i.e., IgG1, IgG2, IgG3 and IgG4). In some embodiments, one or both pairs of Fc domains are derived from IgG1. In some embodiments, one or both pairs of Fc domains are derived from IgG2, IgG3, or IgG4. In some embodiments, the Fc region comprises CH2 and CH3 domains derived from IgG1. In some embodiments, the Fc region comprises CH2 and CH3 domains derived from IgG2. In some embodiments, the Fc region comprises CH2 and CH3 domains derived from IgG3. In some embodiments, the Fc region comprises CH2 and CH3 domains derived from IgG4.

The two Fc domains within the Fc region of an engineered antigen-binding molecule can be the same or different from one another. In a native antibody the Fc domains are typically identical, but for the purpose of producing molecules with different binding domains or other asymmetries, the Fc domains might advantageously be different to allow for heterodimerization, as described in Section 6.2.2.2 below.

Native IgG molecules have hinge domains between CH1 and CH2 domains. Exemplary hinge domain sequences are set forth in Table A below.

TABLE A
IgG Hinge Domain Sequences

| | | SEQ ID |
|---|---|---|
| Hinge Domain | Sequence | NO |
| hIgG1 Hinge | EPKSCDKTHTCPPCP | 3 |
| hIgG2 Hinge | ERKCCVECPPCP | 4 |
| hIgG4 Hinge | ESKYGPPCPSCP | 5 |

It will be appreciated that the heavy chain constant domains for use in producing an Fc region for the engineered antigen-binding molecules of the present disclosure may include variants of the naturally occurring constant domains described above. Such variants may comprise one or more amino acid variations compared to wild type constant domains. In one example the Fc region of the present disclosure comprises at least one constant domain that varies in sequence from the wild type constant domain. It will be appreciated that the variant constant domains may be longer or shorter than the wild type constant domain. Preferably the variant constant domains are at least 60% identical or similar to a wild type constant domain. In another example the variant constant domains are at least 70% identical or similar. In another example the variant constant domains are at least 80% identical or similar. In another example the variant constant domains are at least 90% identical or similar. In another example the variant constant domains are at least 95% identical or similar.

The Fc domains that are incorporated into the engineered antigen-binding molecules of the present disclosure may comprise one or more modifications that alter the functional properties of the proteins, for example, binding to Fc-receptors such as FcRn or leukocyte receptors, binding to complement, modified disulfide bond architecture, or altered glycosylation patterns. Exemplary Fc modifications that alter effector function are described in Section 6.2.2.1.

The Fc domains can also be altered to include modifications that improve manufacturability of engineered antigen-binding molecules, for example by allowing heterodimerization, which is the preferential pairing of non-identical Fc domains over identical Fc domains. Heterodimerization permits the production of multivalent engineered antigen-binding molecules in which different polypeptide components are connected to one another by an Fc region containing Fc domains that differ in sequence. Examples of heterodimerization strategies are exemplified in Section 6.2.2.2.

It will be appreciated that any of the modifications mentioned above can be combined in any suitable manner to achieve the desired functional properties and/or combined with other modifications to alter the properties of the engineered antigen-binding molecules.

Example Fc domain sequences are provided in Table F-1, below. In some embodiments, an Fc domain comprises an amino acid sequence having at least 90%, at least 95%, at least 98%, at least 99%, or 100% sequence identity with an amino acid sequence set forth in Table F-1.

TABLE F-1
Fc Sequences

| | | SEQ |
|---|---|---|
| Fc | Sequence | ID NO |
| hIgG1 Fc | EPKSCDKTHTCPPCPAPELLGGPSVFLFPPKPKDTLMISRTPEVTCVVVDVS | 6 |
| (amino acids 99- | HEDPEVKFNWYVDGVEVHNAKTKPREEQYNSTYRVVSVLTVLHQDWLNGKEY | |
| 330 of UniprotKB | KCKVSNKALPAPIEKTISKAKGQPREPQVYTLPPSRDELTKNQVSLTCLVKG | |
| P01857-1) | FYPSDIAVEWESNGQPENNYKTTPPVLDSDGSFFLYSKLTVDKSRWQQGNVF | |
| | SCSVMHEALHNHYTQKSLSLSPGK | |
| hIgG2 Fc | ERKCCVECPPCPAPPVAGPSVFLFPPKPKDTLMISRTPEVTCVVVDVSHEDP | 7 |
| (amino acids 99- | EVQFNWYVDGVEVHNAKTKPREEQFNSTFRVVSVLTVVHQDWLNGKEYKCKV | |
| 326 of UniprotKB | SNKGLPAPIEKTISKTKGQPREPQVYTLPPSREEMTKNQVSLTCLVKGFYPS | |
| P01859-1) | DISVEWESNGQPENNYKTTPPMLDSDGSFFLYSKLTVDKSRWQQGNVFSCSV | |
| | MHEALHNHYTQKSLSLSPGK | |
| hIgG3 Fc | ELKTPLGDTTHTCPRCPEPKSCDTPPPCPRCPEPKSCDTPPPCPRCPEPKSC | 8 |
| (amino acids 99- | DTPPPCPRCPAPELLGGPSVFLFPPKPKDTLMISRTPEVTCVVVDVSHEDPE | |
| 377 of UniprotKB | VQFKWYVDGVEVHNAKTKPREEQYNSTFRVVSVLTVLHQDWLNGKEYKCKVS | |
| P01860-1) | NKALPAPIEKTISKTKGQPREPQVYTLPPSREEMTKNQVSLTCLVKGFYPSD | |
| | IAVEWESSGQPENNYNTTPPMLDSDGSFFLYSKLTVDKSRWQQGNIFSCSVM | |
| | HEALHNRFTQKSLSLSPGK | |
| hIgG4 Fc | ESKYGPPCPSCPAPEFLGGPSVFLFPPKPKDTLMISRTPEVTCVVVDVSQED | 9 |
| (amino acids 99- | PEVQFNWYVDGVEVHNAKTKPREEQFNSTYRVVSVLTVLHQDWLNGKEYKCK | |
| 327 of UniprotKB | VSNKGLPSSIEKTISKAKGQPREPQVYTLPPSQEEMTKNQVSLTCLVKGFYP | |
| P01861-1) | SDIAVEWESNGQPENNYKTTPPVLDSDGSFFLYSRLTVDKSRWQEGNVFSCS | |
| | VMHEALHNHYTQKSLSLSLGK | |
| hIgG4s Fc | ESKYGPPCPPCPAPPVAGPSVFLFPPKPKDTLMISRTPEVTCVVVDVSQEDP | 10 |
| | EVQFNWYVDGVEVHNAKTKPREEQFNSTYRVVSVLTVLHQDWLNGKEYKCKV | |
| | SNKGLPSSIEKTISKAKGQPREPQVYTLPPSQEEMTKNQVSLTCLVKGFYPS | |
| | DIAVEWESNGQPENNYKTTPPVLDSDGSFFLYSRLTVDKSRWQEGNVFSCSV | |
| | MHEALHNHYTQKSLSLSLGK | |
| hIgG1 PVA Fc | EPKSCDKTHTCPPCPAPPVAGPSVFLFPPKPKDTLMISRTPEVTCVVVDVSH | 11 |
| | EDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCLVKGF | |
| | YPSDIAVEWESNGQPENNYKTTPPVLDSDGSFFLYSKLTVDKSRWQQGNVFS | |
| | CSVMHEALHNHYTQKSLSLSPGK | |
| hIgG1 PVA/P329A | EPKSCDKTHTCPPCPAPPVAGPSVFLFPPKPKDTLMISRTPEVTCVVVDVSH | 12 |
| Fc | EDPEVKFNWYVDGVEVHNAKTKPREEQYNSTYRVVSVLTVLHQDWLNGKEYK | |
| | CKVSNKALAAPIEKTISKAKGQPREPQVYTLPPSRDELTKNQVSLTCLVKGF | |
| | YPSDIAVEWESNGQPENNYKTTPPVLDSDGSFFLYSKLTVDKSRWQQGNVFS | |
| | CSVMHEALHNHYTQKSLSLSPGK | |
| hIgG1 LALAPG Fc | EPKSCDKTHTCPPCPAPEAAGGPSVFLFPPKPKDTLMISRTPEVTCVVVDVS | 13 |
| | HEDPEVKFNWYVDGVEVHNAKTKPREEQYNSTYRVVSVLTVLHQDWLNGKEY | |
| | KCKVSNKALGAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQGNVF | |
| | SCSVMHEALHNHYTQKSLSLSPGK | |
| hIgG1 PVA star | EPKSCDKTHTCPPCPAPPVAGPSVFLFPPKPKDTLMISRTPEVTCVVVDVSH | 14 |
| | EDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCLVKGF | |
| | YPSDIAVEWESNGQPENNYKTTPPVLDSDGSFFLYSKLTVDKSRWQQGNVFS | |
| | CSVMHEALHNRFTQKSLSLSPGK | |
| hIgG1s | DKKVEPKSCDKTHTCPPCPAPPVAGPSVFLFPPKPKDTLMISRTPEVTCVVV | 15 |
| | DVSQEDPEVQFNWYVDGVEVHNAKTKPREEQFNSTYRVVSVLTVLHQDWLNG | |
| | KEYKCKVSNKGLPSSIEKTISKAKGQPREPQVYTLPPSRDELTKNQVSLTCL | |
| | VKGFYPSDIAVEWESNGQPENNYKTTPPVLDSDGSFFLYSKLTVDKSRWQQG | |
| | NVFSCSVMHEALHNHYTQKSLSLSPGK | |
| hIgG1 N180G, | EPKSCDKTHTCPPCPAPELLGGPSVFLFPPKPKDTLMISRTPEVTCVVVDVS | 16 |
| also referred | HEDPEVKFNWYVDGVEVHNAKTKPREEQYGSTYRVVSVLTVLHQDWLNGKEY | |
| to as | KCKVSNKALPAPIEKTISKAKGQPREPQVYTLPPSRDELTKNQVSLTCLVKG | |
| N297G | FYPSDIAVEWESNGQPENNYKTTPPVLDSDGSFFLYSKLTVDKSRWQQGNVF | |
| | SCSVMHEALHNHYTQKSLSLSPGK | |
| hIgG2 variant | ERKCCVECPPCPAPPVAGPSVFLFPPKPKDTLMISRTPEVTCVVVDVSHEDP | 17 |
| | EVQFNWYVDGVEVHNAKTKPREEQFNSTFRVVSVLTVVHQDWLNGKEYKCKV | |
| | SNKGLPAPIEKTISKTKGQPREPQVYTLPPSREEMTKNQVSLTCLVKGFYPS | |
| | DIAVEWESNGQPENNYKTTPPMLDSDGSFFLYSKLTVDKSRWQQGNVFSCSV | |
| | MHEALHNHYTQKSLSLSPGK | |
| hIgG4 S108P | ESKYGPPCPPCPAPEFLGGPSVFLFPPKPKDTLMISRTPEVTCVVVDVSQED | 18 |
| | PEVQFNWYVDGVEVHNAKTKPREEQFNSTYRVVSVLTVLHQDWLNGKEYKCK | |
| | VSNKGLPSSIEKTISKAKGQPREPQVYTLPPSQEEMTKNQVSLTCLVKGFYP | |
| | SDIAVEWESNGQPENNYKTTPPVLDSDGSFFLYSRLTVDKSRWQEGNVFSCS | |
| | VMHEALHNHYTQKSLSLSLGK | |
| IgG1PVA_hinge- | EPKSCDKTHTCPPCPAPPVAGPSVFLFPPKPKDTLMISRTPEVTCVVVDVSH | 19 |
| Fc knob | EDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLWCLVKGF | |
| | YPSDIAVEWESNGQPENNYKTTPPVLDSDGSFFLYSKLTVDKSRWQQGNVFS | |
| | CSVMHEALHNHYTQKSLSLSPGK | |
| IgG1PVA_hinge- | EPKSCDKTHTCPPCPAPPVAGPSVFLFPPKPKDTLMISRTPEVTCVVVDVSH | 20 |
| Fc knob_star | EDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLWCLVKGF | |
| | YPSDIAVEWESNGQPENNYKTTPPVLDSDGSFFLYSKLTVDKSRWQQGNVFS | |
| | CSVMHEALHNRFTQKSLSLSPGK | |
| IgG1PVA_hinge- | EPKSCDKTHTCPPCPAPPVAGPSVFLFPPKPKDTLMISRTPEVTCVVVDVSH | 21 |
| Fc knob_Cys | EDPEVKFNWYVDGVEVHNAKTKPREEQYNSTYRVVSVLTVLHQDWLNGKEYK | |
| | CKVSNKALPAPIEKTISKAKGQPREPQVYTLPPCRDELTKNQVSLWCLVKGF | |
| | YPSDIAVEWESNGQPENNYKTTPPVLDSDGSFFLYSKLTVDKSRWQQGNVFS | |
| | CSVMHEALHNHYTQKSLSLSPGK | |
| IgG1PVA_hinge- | EPKSCDKTHTCPPCPAPPVAGPSVFLFPPKPKDTLMISRTPEVTCVVVDVSH | 22 |
| Fc knob_Cys_star | EDPEVKFNWYVDGVEVHNAKTKPREEQYNSTYRVVSVLTVLHQDWLNGKEYK | |
| | CKVSNKALPAPIEKTISKAKGQPREPQVYTLPPCRDELTKNQVSLWCLVKGF | |
| | YPSDIAVEWESNGQPENNYKTTPPVLDSDGSFFLYSKLTVDKSRWQQGNVFS | |
| | CSVMHEALHNRFTQKSLSLSPGK | |
| IgG1PVA_hinge- | EPKSCDKTHTCPPCPAPPVAGPSVFLFPPKPKDTLMISRTPEVTCVVVDVSH | 23 |
| Fc Hole | EDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLSCAVKGF | |
| | YPSDIAVEWESNGQPENNYKTTPPVLDSDGSFFLVSKLTVDKSRWQQGNVFS | |
| | CSVMHEALHNHYTQKSLSLSPGK | |
| IgG1PVA_hinge- | EPKSCDKTHTCPPCPAPPVAGPSVFLFPPKPKDTLMISRTPEVTCVVVDVSH | 24 |
| Fc Hole_star | EDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLSCAVKGF | |
| | YPSDIAVEWESNGQPENNYKTTPPVLDSDGSFFLVSKLTVDKSRWQQGNVFS | |
| | CSVMHEALHNRFTQKSLSLSPGK | |
| IgG1PVA_hinge- | EPKSCDKTHTCPPCPAPPVAGPSVFLFPPKPKDTLMISRTPEVTCVVVDVSH | 25 |
| Fc Hole_Cys | EDPEVKFNWYVDGVEVHNAKTKPREEQYNSTYRVVSVLTVLHQDWLNGKEYK | |
| | CKVSNKALPAPIEKTISKAKGQPREPQVCTLPPSRDELTKNQVSLSCAVKGF | |
| | YPSDIAVEWESNGQPENNYKTTPPVLDSDGSFFLVSKLTVDKSRWQQGNVFS | |
| | CSVMHEALHNHYTQKSLSLSPGK | |
| IgG1PVA_hinge- | EPKSCDKTHTCPPCPAPPVAGPSVFLFPPKPKDTLMISRTPEVTCVVVDVSH | 26 |
| Fc Hole_Cys_star | EDPEVKFNWYVDGVEVHNAKTKPREEQYNSTYRVVSVLTVLHQDWLNGKEYK | |
| | CKVSNKALPAPIEKTISKAKGQPREPQVCTLPPSRDELTKNQVSLSCAVKGF | |
| | YPSDIAVEWESNGQPENNYKTTPPVLDSDGSFFLVSKLTVDKSRWQQGNVFS | |
| | CSVMHEALHNRFTQKSLSLSPGK | |

6.2.2.1. Fc Domains with Altered Effector Function

In some embodiments, the Fc domain comprises one or more amino acid substitutions that reduce binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the Fc domain or the Fc region (e.g., one or both Fc domains of an engineered antigen-binding molecule that can associate to form an Fc region) comprises an amino acid substitution at a position selected from the group of E233, L234, L235, N297, P331 and P329 (numberings according to Kabat EU index). In a more specific embodiment, the Fc domain or the Fc region comprises an amino acid substitution at a position selected from the group of L234, L235 and P329 (numberings according to Kabat EU index). In some embodiments, the Fc domain or the Fc region comprises the amino acid substitutions L234A and L235A (numberings according to Kabat EU index). In one such embodiment, the Fc domain or region is an Igd Fc domain or region, particularly a human Igd Fc domain or region. In one embodiment, the Fc domain or the Fc region comprises an amino acid substitution at position P329. In a more specific embodiment, the amino acid substitution is P329A or P329G, particularly P329G (numberings according to Kabat EU index). In one embodiment, the Fc domain or the Fc region comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain or the Fc region comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”).

Typically, the same one or more amino acid substitution is present in each of the two Fc domains of an Fc region. Thus, in a particular embodiment, each Fc domain of the Fc region comprises the amino acid substitutions L234A, L235A and P329G (Kabat EU index numbering), i.e. in each of the first and the second Fc domains in the Fc region the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one embodiment, the Fc domain is an IgG1 Fc domain, particularly a human IgG1 Fc domain. In some embodiments, the IgG1 Fc domain is a variant IgG1 comprising D265A, N297A mutations (EU numbering) to reduce effector function.

In some embodiments, the Fc domain has one or more modifications that increase binding to FcγRIIB. In some embodiments, one or both Fc regions have S239D and 1332E amino acid substitutions (Kabat EU index numbering). In some embodiments, one or both Fc regions have S239D, A330L, and 1332E amino acid substitutions (Kabat EU index numbering). In some embodiments, one or both Fc regions have G236A amino acid substitutions (Kabat EU index numbering). In some embodiments, one or both Fc regions have G236A, S239D, and 1332E amino acid substitutions (Kabat EU index numbering). In some embodiments, one or both Fc regions have G236A, S239D, A330L, and 1332E amino acid substitutions (Kabat EU index numbering). In some embodiments, one or both Fc regions have F243L, R292P, Y300L, V305I, and P396L amino acid substitutions (Kabat EU index numbering). In some embodiments, one or both Fc regions have S239D and 1332E amino acid substitutions (Kabat EU index numbering).

In another embodiment, the Fc domain is an IgG4 Fc domain with reduced binding to Fc receptors. Exemplary IgG4 Fc domains with reduced binding to Fc receptors may comprise an amino acid sequence selected from Table C below. In some embodiments, the Fc domain includes only the bolded portion of the sequences shown below:

TABLE C

| | | SEQ |
|---|---|---|
| Fc Domain | Sequence | ID NO |
| SEQ ID NO: 1 of | DKRVESKYGP PCPPCPAPPV AGPSVFLFPP KPKDTLMISR | 27 |
| WO2014/121087 | TPEVTCVVVD VSQEDPEVQF NWYVDGVEVH NAKTKPREEQ | |
| | FNSTYRVVSV LTVLHQDWLN GKEYKCKVSN KGLPSSIEKT | |
| | ISKAKGQPRE PQVYTLPPSQ EEMTKNQVSL TCLVKGFYPS | |
| | DIAVEWESNG QPENNYKTTP PVLDSDGSFF LYSRLTVDKS | |
| | RWQEGNVFSC SVMHEALHNH YTQKSLSLSL GK | |
| SEQ ID NO: 2 of | DKKVEPKSCD KTHTCPPCPA PPVAGPSVFL FPPKPKDTLM | 28 |
| WO2014/121087 | ISRTPEVTCV VVDVSQEDPE VQFNWYVDGV EVHNAKTKPR | |
| | EEQFNSTYRV VSVLTVLHQD WLNGKEYKCK VSNKGLPSSI | |
| | EKTISKAKGQ PREPQVYTLP PSRDELTKNQ VSLTCLVKGF | |
| | YPSDIAVEWE SNGQPENNYK TTPPVLDSDG SFFLYSKLTV | |
| | DKSRWQQGNV FSCSVMHEAL HNHYTQKSLS LSPGK | |
| | ASTKGPSVFP LAPSSKSTSG GTAALGCLVK DYFPEPVTVS | |
| SEQ ID NO: 30 | WNSGALTSGV HTFPAVLQSS GLYSLSSVVT VPSSSLGTQT | 29 |
| of | YICNVNHKPS NTKVDKKVEP KSCDKTHTCP PCPAPPVAGP | |
| WO2014/121087 | SVFLFPPKPK DTLMISRTPE VTCVVVDVSQ EDPEVQFNWY | |
| | VDGVEVHNAK TKPREEQFNS TYRVVSVLTV LHQDWLNGKE | |
| | YKCKVSNKGL PSSIEKTISK AKGQPREPQV YTLPPSRDEL | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPGK | |
| SEQ ID NO: 31 | ASTKGPSVFP LAPCSRSTSE STAALGCLVK DYFPEPVTVS | 30 |
| of | WNSGALTSGV HTFPAVLQSS GLYSLSSVVT VPSSSLGTKT | |
| WO2014/121087 | YTCNVDHKPS NTKVDKRVES KYGPPCPPCP APPVAGPSVF | |
| | LFPPKPKDTL MISRTPEVTC VVVDVSQEDP EVQFNWYVDG | |
| | VEVHNAKTKP REEQFNSTYR VVSVLTVLHQ DWLNGKEYKC | |
| | KVSNKGLPSS IEKTISKAKG QPREPQVYTL PPSQEEMTKN | |
| | QVSLTCLVKG FYPSDIAVEW ESNGQPENNY KTTPPVLDSD | |
| | GSFFLYSRLT VDKSRWQEGN VFSCSVMHEA LHNHYTQKSL | |
| | SLSLGK | |
| SEQ ID NO: 37 | ASTKGPSVFP LAPSSKSTSG GTAALGCLVK DYFPEPVTVS | 31 |
| of | WNSGALTSGV HTFPAVLQSS GLYSLSSVVT VPSSSLGTQT | |
| WO2014/121087 | YICNVNHKPS NTKVDKKVEP KSCDKTHTCP PCPAPPVAGP | |
| | SVFLFPPKPK DTLMISRTPE VTCVVVDVSQ EDPEVQFNWY | |
| | VDGVEVHNAK TKPREEQFNS TYRVVSVLTV LHQDWLNGKE | |
| | YKCKVSNKGL PSSIEKTISK AKGQPREPQV YTLPPSRDEL | |
| | TKNQVSLTCL VKGFYPSDIA VEWESNGQPE NNYKTTPPVL | |
| | DSDGSFFLYS KLTVDKSRWQ QGNVFSCSVM HEALHNRFTQ | |
| | KSLSLSPGK | |
| SEQ ID NO: 38 | ASTKGPSVFP LAPCSRSTSE STAALGCLVK DYFPEPVTVS | 32 |
| of | WNSGALTSGV HTFPAVLQSS GLYSLSSVVT VPSSSLGTKT | |
| WO2014/121087 | YTCNVDHKPS NTKVDKRVES KYGPPCPPCP APPVAGPSVF | |
| | LFPPKPKDTL MISRTPEVTC VVVDVSQEDP EVQFNWYVDG | |
| | VEVHNAKTKP REEQFNSTYR VVSVLTVLHQ DWLNGKEYKC | |
| | KVSNKGLPSS IEKTISKAKG QPREPQVYTL PPSQEEMTKN | |
| | QVSLTCLVKG FYPSDIAVEW ESNGQPENNY KTTPPVLDSD | |
| | GSFFLYSRLT VDKSRWQEGN VFSCSVMHEA LHNRFTQKSL | |
| | SLSLGK | |

In a particular embodiment, the IgG4 with reduced effector function comprises the bolded portion of the amino acid sequence of SEQ ID NO:30 (SEQ ID NO:31 of WO2014/121087), sometimes referred to herein as IgG4s or hIgG4s.

For heterodimeric Fc regions, it is possible to incorporate a combination of the variant IgG4 Fc sequences set forth above, for example an Fc region comprising an Fc domain comprising the amino acid sequence of SEQ ID NO:29 (SEQ ID NO:30 of WO2014/121087) (or the bolded portion thereof) and an Fc domain comprising the amino acid sequence of SEQ ID NO:31 (SEQ ID NO:37 of WO2014/121087) (or the bolded portion thereof) or an Fc region comprising an Fc domain comprising the amino acid sequence of SEQ ID NO:30 (SEQ ID NO:31 of WO2014/121087) (or the bolded portion thereof) and an Fc domain comprising the amino acid sequence of SEQ ID NO:32 (SEQ ID NO:38 of WO2014/121087) (or the bolded portion thereof).

### 6.2.2.2. Fc Heterodimerization Variants

Certain engineered antigen-binding molecules entail dimerization between two Fc domains that, unlike a native immunoglobulin, are operably linked to non-identical N-terminal regions, e.g., one Fc domain connected to a first targeting moiety and the other Fc domain connected to a second, different, targeting moiety. Inadequate heterodimerization of two Fc domains to form an Fc region can be an obstacle for increasing the yield of desired heterodimeric molecules and represents challenges for purification. A variety of approaches available in the art can be used in for enhancing dimerization of Fc domains that might be present in the engineered antigen-binding molecules of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO 2009/089004A1.

The present disclosure provides engineered molecules comprising Fc heterodimers, i.e., Fc regions comprising heterologous, non-identical Fc domains. Typically, each Fc domain in the Fc heterodimer comprises a CH3 domain of an antibody. The CH3 domains are derived from the constant region of an antibody of any IgG subclass, as described above.

Heterodimerization of the two different heavy chains at CH3 domains give rise to the desired engineered antigen-binding molecule, while homodimerization of identical heavy chains will reduce yield of the desired engineered antigen-binding molecule. Thus, in some embodiments, the polypeptides that associate to form an engineered antigen-binding molecule of the disclosure will contain CH3 domains with modifications that favor heterodimeric association relative to unmodified Fc domains.

In a specific embodiment said modification promoting the formation of Fc heterodimers is a so-called “knob-into-hole” or “knob-in-hole” modification, comprising a “knob” modification in one of the Fc domains and a “hole” modification in the other Fc domain. The knob-into-hole technology is described e.g., in U.S. Pat. Nos. 5,731,168; 7,695,936; Ridgway et al., 1996, Prot Eng 9:617-621, and Carter, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine(S), threonine (T), and valine (V). The protuberance and cavity can be made by altering the nucleic acid encoding the polypeptides, e.g., by site-specific mutagenesis, or by peptide synthesis. An exemplary substitution is Y470T.

In a specific such embodiment, in the first Fc domain the threonine residue at position 366 is replaced with a tryptophan residue (T366W), and in the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second Fc domain additionally the tyrosine residue at position 349 is replaced by a cysteine residue (Y349C) (numbering according to Kabat EU index). In a particular embodiment, the first Fc domain comprises the amino acid substitutions S354C and T366W, and the second Fc domain comprises the amino acid substitutions Y349C, T366S, L368A and Y407V (numbering according to Kabat EU index).

In some embodiments, electrostatic steering (e.g., as described in Gunasekaran et al., 2010, J Biol Chem 285(25): 19637-46) can be used to promote the association of the first and the second Fc domains of the Fc region.

As an alternative, or in addition, to the use of Fc domains that are modified to promote heterodimerization, an Fc domain can be modified to allow a purification strategy that enables selections of Fc heterodimers. In one such embodiment, one polypeptide comprises a modified Fc domain that abrogates its binding to Protein A, thus enabling a purification method that yields a heterodimeric protein. See, for example, U.S. Pat. No. 8,586,713. As such, engineered antigen-binding molecule may comprise a first CH3 domain and a second Ig CH3 domain, wherein the first and second Ig CH3 domains differ from one another by at least one amino acid, and wherein at least one amino acid difference reduces binding of the engineered antigen-binding to Protein A as compared to a corresponding engineered antigen-binding protein lacking the amino acid difference. In one embodiment, the first CH3 domain binds Protein A and the second CH3 domain contains a mutation/modification that reduces or abolishes Protein A binding such as an H95R modification (by IMGT exon numbering; H435R by EU numbering). The second CH3 may further comprise a Y96F modification (by IMGT; Y436F by EU). This class of modifications is referred to herein as “star” mutations.

In some embodiments, the Fc can contain one or more mutations (e.g., knob and hole mutations) to facilitate heterodimerization as well as star mutations to facilitate purification.

### 6.2.2.3. Hinge Domains

The engineered antigen-binding molecules of the disclosure can comprise an Fc domain comprising a hinge domain at its N-terminus. The hinge region can be a native or a modified hinge region. Hinge regions are typically found at the N-termini of Fc regions. The term “hinge domain”, unless the context dictates otherwise, refers to a naturally or non-naturally occurring hinge sequence that in the context of a single or monomeric polypeptide chain is a monomeric hinge domain and in the context of a dimeric polypeptide (e.g., a homodimeric or heterodimeric multispecific binding molecule formed by the association of two Fc domains) can comprise two associated hinge sequences on separate polypeptide chains. Sometimes, the two associated hinge sequences are referred to as a “hinge region”. In certain embodiments of engineered antigen-binding molecules of the disclosure, additional iterations of hinge regions may be incorporated into the polypeptide sequence.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions may comprise a complete hinge region derived from an antibody of a different class or subclass from that of the heavy chain Fc domain or Fc region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO 99/15549, WO 2005/003170, WO 2005/003169, WO 2005/003170, WO 98/25971 and WO 2005/003171 and these are incorporated herein by reference.

In one embodiment, an engineered antigen-binding molecule of the disclosure comprises an Fc region in which one or both Fc domains possesses an intact hinge domain at its N-terminus.

In various embodiments, positions 233-236 within a hinge region may be G, G, G and unoccupied; G, G, unoccupied, and unoccupied; G, unoccupied, unoccupied, and unoccupied; or all unoccupied, with positions numbered by EU numbering.

In some embodiments, the engineered antigen-binding molecules of the disclosure comprise a modified hinge region that reduces binding affinity for an Fcγ receptor relative to a wild-type hinge region of the same isotype (e.g., human IgG1 or human IgG4).

In one embodiment, the engineered antigen-binding molecules of the disclosure comprise an Fc region in which each Fc domain possesses an intact hinge domain at its N-terminus, where each Fc domain and hinge domain is derived from IgG4 and each hinge domain comprises the modified sequence CPPC (SEQ ID NO:43). The core hinge region of human IgG4 contains the sequence CPSC (SEQ ID NO:44) compared to IgG1 that contains the sequence CPPC (SEQ ID NO:43). The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angel et al., 1993, Mol Immunol 30(1):105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

### 6.2.2.3.1 Chimeric Hinge Sequences

The hinge domain can be a chimeric hinge domain (also “chimeric hinge region”).

For example, a chimeric hinge domain may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region.

In particular embodiments, a chimeric hinge region comprises the amino acid sequence EPKSCDKTHTCPPCPAPPVA (SEQ ID NO:45) (previously disclosed as SEQ ID NO: 8 of WO2014/121087, which is incorporated by reference in its entirety herein) or ESKYGPPCPPCPAPPVA (SEQ ID NO:46) (previously disclosed as SEQ ID NO:9 of WO2014/121087). Such chimeric hinge sequences can be suitably linked to an IgG4 CH2 region (for example by incorporation into an IgG4 Fc domain, for example a human or murine Fc domain, which can be further modified in the CH2 and/or CH3 domain to reduce effector function, for example as described in Section 6.2.2.3.2).

6.2.2.3.2 Hinge Sequences with Reduced Effector Function

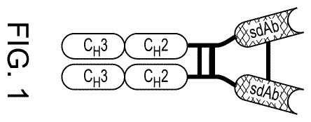

In further embodiments, the hinge region can be modified to reduce effector function, for example as described in WO2016161010A2, which is incorporated by reference in its entirety herein. In various embodiments, the positions 233-236 of the modified hinge region are G, G, G and unoccupied; G, G, unoccupied, and unoccupied; G, unoccupied, unoccupied, and unoccupied; or all unoccupied, with positions numbered by EU numbering (as shown in FIG. 1 of WO2016161010A2). These segments can be represented as GGG-, GG--, G--- or ---- with “-” representing an unoccupied position.

Position 236 is unoccupied in canonical human IgG2 but is occupied by in other canonical human IgG isotypes. Positions 233-235 are occupied by residues other than G in all four human isotypes (as shown in FIG. 1 of WO2016161010A2).

The hinge modification within positions 233-236 can be combined with position 228 being occupied by P. Position 228 is naturally occupied by P in human IgG1 and IgG2 but is occupied by S in human IgG4 and R in human IgG3. An S228P mutation in an IgG4 antibody is advantageous in stabilizing an IgG4 antibody and reducing exchange of heavy chain light chain pairs between exogenous and endogenous antibodies. Preferably positions 226-229 are occupied by C, P, P and C respectively.

Exemplary hinge regions have residues 226-236, sometimes referred to as middle (or core) and lower hinge, occupied by the modified hinge sequences designated GGG-(233-236), GG-(233-236), G---(233-236) and no G (233-236). Optionally, the hinge domain amino acid sequence comprises CPPCPAPGGG-GPSVF (SEQ ID NO:47) (previously disclosed as SEQ ID NO:1 of WO2016161010A2), CPPCPAPGG-GPSVF (SEQ ID NO: 438) (previously disclosed as SEQ ID NO:2 of WO2016161010A2), CPPCPAPG---GPSVF (SEQ ID NO:49) (previously disclosed as SEQ ID NO:3 of WO2016161010A2), or CPPCPAP----GPSVF (SEQ ID NO:50) (previously disclosed as SEQ ID NO:4 of WO2016161010A2).

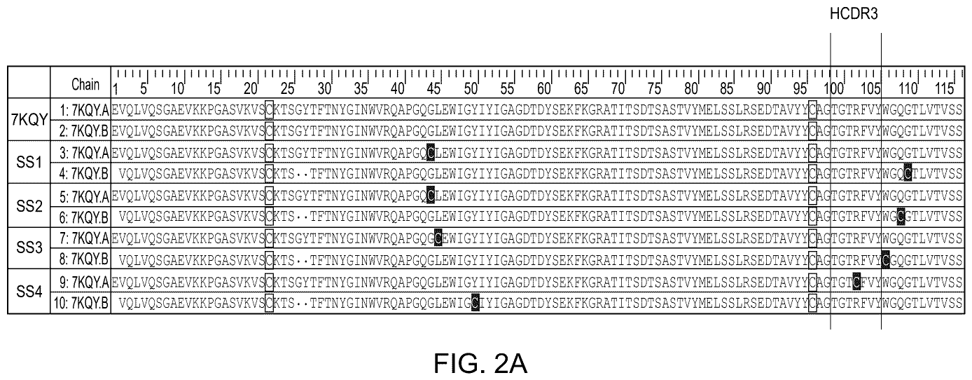

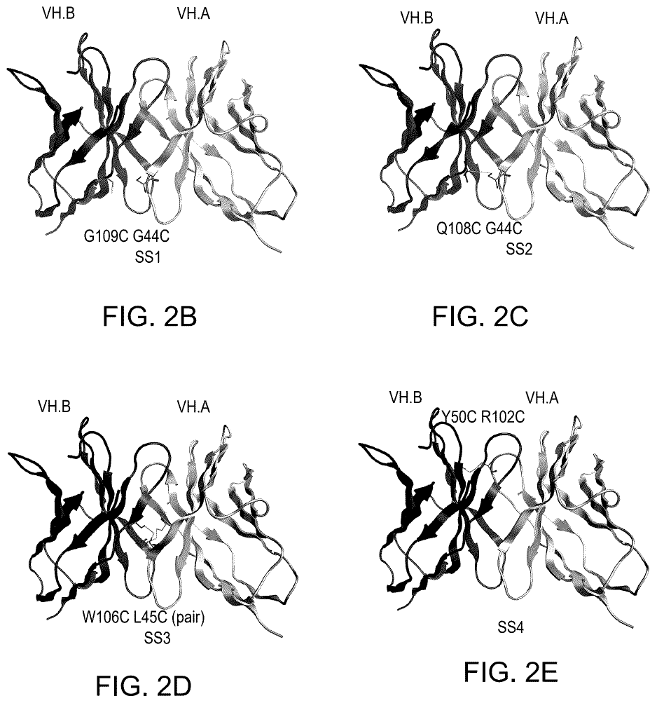

The modified hinge regions described above can be incorporated into a heavy chain constant region, which typically includes CH2 and CH3 domains, and which may have an additional hinge segment (e.g., an upper hinge) flanking the designated region. Such additional constant region segments present are typically of the same isotype, preferably a human isotype, although can be hybrids of different isotypes. The isotype of such additional human constant regions segments is preferably human IgG4 but can also be human IgG1, IgG2, or IgG3 or hybrids thereof in which domains are of different isotypes. Exemplary sequences of human IgG1, IgG2 and IgG4 are shown in FIGS. 2-4 of WO2016161010A2.

In specific embodiments, the modified hinge sequences can be linked to an IgG4 CH2 region (for example by incorporation into an IgG4 Fc domain, for example a human or murine Fc domain, which can be further modified in the CH2 and/or CH3 domain to reduce effector function, for example as described in Section 6.2.2.1).

### 6.2.3. Targeting Moieties

Engineered antigen-binding molecules described herein may include two or more binding moieties. The one or more binding moieties may include, for example, two or more sdAb domains.

Targeting moieties included in the engineered antigen-binding molecules of the present disclosure may specifically bind to one or more target molecules. Target molecules may include, for example, cell surface receptors that can be agonized by an antibody having targeting moieties that bind to the target molecule. For example, the one or more target molecules may be CD40, OX40, GITR, 4-1BB, CD27, HVEM, CD30, leptin receptor (LEPR), insulin-like growth factor 1 receptor (IGF1R), erythropoietin (EPO) receptor, G-CSF receptor, leptin receptor (LEPR), thrombopoietin receptor (TPOR), growth hormone receptor (GHR), or prolactin receptor (PRLR).

In some embodiments, one or more targeting moieties of an engineered antigen-binding molecule of the present disclosure selectively binds a member of the TNF receptor superfamily, also referred to herein as TNF family receptors. In some embodiments, the TNF family receptor is CD40, OX40, glucocorticoid-induced TNFR-related protein (GITR), 4-1BB, CD27, herpesvirus entry mediator (HVEM), or CD30. Activation of a TNF family receptor may be useful in methods of activating immune cells and treating diseases such as cancer.

In some embodiments, one or more targeting moieties of an engineered antigen-binding molecule of the present disclosure selectively binds a homodimeric class I cytokine receptor. In some embodiments, the homodimeric class I cytokine receptor is EPO receptor, G-CSF receptor, leptin receptor (LEPR), thrombopoietin receptor (TPOR), growth hormone receptor (GHR), or prolactin receptor (PRLR). Embodiments of engineered antigen-binding molecules that bind to an EPO receptor target molecule may include targeting moieties that bind one or more epitopes of the EpoR protein. Embodiments of engineered antigen-binding molecules that bind to a G-CSF receptor target molecule (a homodimer receptor) may include targeting moieties that bind one or more epitopes of the G-CSFR protein. Embodiments of engineered antigen-binding molecules that bind to a leptin receptor target molecule (a homodimer receptor) may include targeting moieties that bind one or more epitopes of the LEPR protein. Embodiments of engineered antigen-binding molecules that bind to a thrombopoietin receptor target molecule (a homodimer receptor) may include targeting moieties that bind one or more epitopes of the TPOR protein. Embodiments of engineered antigen-binding molecules that bind to a growth hormone receptor target molecule (a homodimer receptor) may include targeting moieties that bind one or more epitopes of the GHR protein. Embodiments of engineered antigen-binding molecules that bind to a prolactin receptor target molecule (a homodimer receptor) may include targeting moieties that bind one or more epitopes of the PRLR protein.

Exemplary targeting moieties that may be used in embodiments of engineered antigen-binding molecules include the targeting moieties from the antibodies set forth in Table B below.

TABLE B
Exemplary Single Domain Antibody (sdAb) Amino Acid Sequences

| Target | Reference | Sequence |
|---|---|---|
| OX40 | 1A06 sdAb: | EVQLVQSGGGLVQPGGSLTLSCVASGIILSHNEMRWYRQN |
| | SEQ ID NO: 16 of PCT | PGKPRDLVAGITSAAYTYYGDFVKGRFTISRDNARNTAYL |
| | Publication No. WO | QMNSLNPGDTGNYYCEVSDGDNQYWGQGTQATVSS (SEQ |
| | 2017/123673 | ID NO: 51) |
| OX40 | 2H06 sdAb: | QLQLQESGGGLVQPGGSLTLSCVASGIILSHNEMRWYRQN |
| | SEQ ID NO: 17 of PCT | PGEPRDLVAGITSAAYTYYGDFVKGRFTISRDNAKNTAYL |
| | Publication No. WO | QMNRLSPEDTGNYYCEVSDGDNQYWGQGTQVTVSS (SEQ |
| | 2017/123673 | ID NO: 52) |
| OX40 | 2E4 sdAb: | QVQLQQSGGGLVQPGGSLSLSCVASGIILSHNEMRWYRQN |
| | SEQ ID NO: 18 of PCT | PGKPRDLVAGITSAAYTYYGDFVKGRFTISRDNAKNTAYL |
| | Publication No. WO | QMDRLNPEDTGNYYCEVSDGDNRYWGQGTQATV (SEQ |
| | 2017/123673 | ID NO: 53) |
| OX40 | 2C09 sdAb: | QVQLQQSGGGLVQPGGSLTLSCVASGIILSHNEMRWYRQN |
| | SEQ ID NO: 19 of PCT | PGKPRDLVAGITSAAYTYYGDFFKGRFTISRDNAKNTAYL |
| | Publication No. WO | QMNRLNPEDTGNYYCEVSDGDIQYWGQGTQATVSS (SEQ |
| | 2017/123673 | ID NO: 54) |
| OX40 | 2E10 sdAb: | QVQLVQSGGGLVQPGGSLTLSCAASGIILSHNEMRWYRQN |
| | SEQ ID NO: 20 of PCT | PGKPRDLVAGITGLDYTYYGDFVKGRFTISRDNAMNTAYL |
| | Publication No. WO | QMDSLNPEDTGNYYCEVSDGDNQYWGQGTQVTVSS (SEQ |
| | 2017/123673 | ID NO: 55) |
| OX40 | 13-5 sdAb: | QVQLQESGGGLVQPGGSLRLSCAASGFTFSDAFMYWVRQA |
| | SEQ ID NO: 21 of PCT | PGKELEWVSSISNRGLKTAYAESVKGRLTISRDNTKNTLY |
| | Publication No. WO | LEMNSLKPEDTGMYYCSRDVGGDLRGQGTQVTVSS (SEQ |
| | 2017/123673 | ID NO: 56) |
| OX40 | 1D10 sdAb: | QVQLQESGGGLVQPGGSLRLSCAASGFTFSDAFMYWVRQA |
| | SEQ ID NO: 22 of PCT | PGKELEWVSSISNRGLKTAYKESVKGRFTISRDNTKNMLF |
| | Publication No. WO | LEMNRLEPEDTGLYYCSRDVDGDFRGQGTQVTV (SEQ |
| | 2017/123673 | ID NO: 57) |
| OX40 | 3E11 sdAb: | QVQLQESGGGLVQPGGSLRLSCAASGFTFSDAFMYWVRQA |
| | SEQ ID NO: 23 of PCT | PGKELEWVSSISNRGLKTAYAESVKGRFTISRDNTKNTLY |
| | Publication No. WO | LQMSSLKPEDTAVYYCATESIGGNGSPYFDLWGQGTQVTV |
| | 2017/123673 | KP (SEQ ID NO: 58) |
| OX40 | 3G9 sdAb: | QVQLQESGGGLVQPGGSLRLSCAASGFTFSDAFMYWVRQA |
| | SEQ ID NO: 24 of PCT | PGKELEWVSSISNRGLKTAYAESVKGRFTISRDNTKNTLY |
| | Publication No. WO | LQMSSLKPEDTAVYYCATESIGSNGSPYFDLWGQGTQVTV |
| | 2017/123673 | KP (SEQ ID NO: 59) |
| OX40 | G3 sdAb: | QVQLQQSGGGLVQPGGSLRLSCAASGFTFSDAFMYWVRQA |
| | SEQ ID NO: 25 of PCT | PGKELEWVSSISNRGLKTAYKESVKGRFTISRDNTKNMLF |
| | Publication No. WO | LEMNRLEPEDTGLYYCVEGDWNLGPRGQGTQVTVKP |
| | 2017/123673 | (SEQ ID NO: 60) |
| OX40 | hzG3v1 sdAb: | EVQLLESGGGEVQPGGSLRLSCAASGFTFSDAFMYWVRQA |
| | SEQ ID NO: 377 of | PGKGLEWVSSISNRGLKTAYAESVKGRFTISRDNAKNTLY |
| | PCT Publication No. | LQMSSLRAEDTAVYYCVEGDWNLGPRGQGTLVTVKP |
| | WO 2017/123673 | (SEQ ID NO: 61) |
| OX40 | hzG3v2 sdAb: | EVQLLESGGGEVQPGGSLRLSCAASGFTFSDAFMYWVRQA |
| | SEQ ID NO: 378 of | PGKELEWVSSISNRGLKTAYAESVKGRFTISRDNAKNTLY |
| | PCT Publication No. | LQMSSLRAEDTAVYYCVEGDWNLGPRGQGTLVTVKP |
| | WO 2017/123673 | (SEQ ID NO: 62) |
| OX40 | hzG3v3 sdAb: | EVQLLESGGGEVQPGGSLRLSCAASGFTFSDAFMYWVRQA |
| | SEQ ID NO: 379 of | PGKELEWVSSISNRGLKTAYAESVKGRFTISRDNTKNTLY |
| | PCT Publication No. | LQMSSLRAEDTAVYYCVEGDWNLGPRGQGTLVTVKP |
| | WO 2017/123673 | (SEQ ID NO: 63) |
| OX40 | hzG3v4 sdAb: | EVQLLESGGGEVQPGGSLRLSCAASGFTFSDAFMYWVRQA |
| | SEQ ID NO: 380 of | PGKELEWVSSISNRGLKTAYAESVKGRFTISRDNTKNMLF |
| | PCT Publication No. | LEMNRLEPEDTGLYYCVEGDWNLGPRGQGTLVTVKP |
| | WO 2017/123673 | (SEQ ID NO: 64) |
| OX40 | hzG3v5 sdAb: | EVQLLESGGGEVQPGGSLRLSCAASGFTFSDAFMYWVRQA |
| | SEQ ID NO: 381 of | PGKELEWVSSISNRGLKTAYAESVKGRFTISRDNTKNTLF |
| | PCT Publication No. | LEMNRLEPEDTGLYYCVEGDWNLGPRGQGTLVTVKP |
| | WO 2017/123673 | (SEQ ID NO: 65) |
| OX40 | hzG3v6 sdAb: | EVQLLESGGGEVQPGGSLRLSCAASGFTFSDAFMYWVRQA |
| | SEQ ID NO: 382 of | PGKELEWVSSISNRGLKTAYKESVKGRFTISRDNTKNMLF |
| | PCT Publication No. | LEMNRLEPEDTGLYYCVEGDWNLGPRGQGTLVTVKP |
| | WO 2017/123673 | (SEQ ID NO: 66) |
| OX40 | hzG3v7 sdAb: | EVQLLESGGGEVQPGGSLRLSCAASGFTFSDAFMYWVRQA |
| | SEQ ID NO: 383 of | PGKELEWVSSISNRGLKTAYKESVKGRFTISRDNTKNTLY |
| | PCT Publication No. | LQMSSLRAEDTAVYYCVEGDWNLGPRGQGTLVTVKP |
| | WO 2017/123673 | (SEQ ID NO: 67) |
| OX40 | hzG3v8 sdAb: | EVQLLESGGGEVQPGGSLRLSCAASGFTFSDAFMYWVRQA |
| | SEQ ID NO: 384 of | PGKGREWVSSISNRGLKTAYAESVKGRFTISRDNAKNTLY |
| | PCT Publication No. | LQMSSLRAEDTAVYYCVEGDWNLGPRGQGTLVTVKP |
| | WO 2017/123673 | (SEQ ID NO: 68) |
| OX40 | hzG3v9 sdAb: | EVQLLESGGGEVQPGGSLRLSCAASGFTFSDAFMYWVRQA |
| | SEQ ID NO: 385 of | PGKEREWVSSISNRGLKTAYAESVKGRFTISRDNAKNTLY |
| | PCT Publication No. | LQMSSLRAEDTAVYYCVEGDWNLGPRGQGTLVTVKP |
| | WO 2017/123673 | (SEQ ID NO: 69) |
| OX40 | 1G4 sdAb: | QVQLQESGGGLVQPGGSLRLSCAASGFTFSDAFMYWVRQA |
| | SEQ ID NO: 26 of PCT | PGKELEWVSSISNRGLKTAYKESVKGRFTISRDNTKNMLY |
| | Publication No. WO | LEMNRLEPEDTGLYYCSRDVDGGFRGQGTQVTVKP (SEQ |
| | 2017/123673 | ID NO: 70) |
| OX40 | A1 sdAb: | QVQLQESGGGLVQPGGSLRLSCAASGFTFNDAFMYWVRQA |
| | SEQ ID NO: 27 of PCT | PGKELEWVSSISNRGLKTAYKESXKGLFTISRDNTKNMLF |
| | Publication No. WO | LEMNRLEPEDXGLYYCSRDVDGDFWGRGTHVTVKP (SEQ |
| | 2017/123673 | ID NO: 71) |
| OX40 | G1 sdAb: | QVQLQESGGGLVQPGGSLRLSCAASGFTFSDAFMYWVRQA |
| | SEQ ID NO: 28 of PCT | PGKELEWVSSISNRGLKTAYAESVKGRFTISRDNTKNTLY |
| | Publication No. WO | LEMNSLKPEDTGMYYCSRDVGGDLRGQGTQVTVKP (SEQ |
| | 2017/123673 | ID NO: 72) |
| OX40 | hz1D10v1 sdAb: | EVQLLESGGGEVQPGGSLRLSCAASGFTFSDAFMYWVRQA |
| | SEQ ID NO: 29 of PCT | PGKGLEWVSSISNRGLKTAYAESVKGRFTISRDNAKNTLY |
| | Publication No. WO | LQMSSLRAEDTAVYYCSRDVDGDFRGQGTLVTVKP (SEQ |
| | 2017/123673 | ID NO: 73) |
| OX40 | 1D11 sdAb: | EVQLLESGGGEVQPGGSLRLSCAASGFTFSDAFMYWVRQA |
| | SEQ ID NO: 386 of | PGKGLEWVSSISNRGLKTAYAESVKGRFTISRDNAKNTLY |
| | PCT Publication No. | LQMSSLRAEDTAVYYCSIDVDGDFRGQGTLVTVKP (SEQ |
| | WO 2017/123673 | ID NO: 74) |
| OX40 | Humanized nanobody | EVQLVESGGGLVQPGGSLRLSCAASGRSFSTYIMGWFRQA |
| | OX40L075: | PGKEREFVATISRSGITTRSADSVKGRFTISRDNSKNTVY |
| | SEQ ID NO: 199 of | LQMNSLRPEDTAVYYCAAGPYVEQTLGLYQTLGPWDYWGQ |
| | PCT Publication No. | GTLVTVSS (SEQ ID NO: 75) |
| | WO 2011/073180 | |
| OX40 | Humanized nanobody | EVQLVESGGGLVQPGGSLRLSCAASGRTFSSIYAKGWFRQ |
| | OX40L024: | APGKEREFVAAISRSGRSTSYADSVKGRFTISRDNAKNTV |
| | SEQ ID NO: 200 of | YLQMNSLRPEDTAVYYCAAVGGATTVTASEWDYWGLGTLV |
| | PCT Publication No. | TVSS (SEQ ID NO: 76) |
| | WO 2011/073180 | |
| OX40 | Humanized nanobody | EVQLVESGGGLVQPGGSLRLSCAASGRTFSSIYAKGWFRQ |
| | OX40L025: | APGKEREFVAAISRSGRSTSYADSVKGRFTISRDNSKNTV |
| | SEQ ID NO: 201 of | YLQMNSLRPEDTAVYYCAAVGGATTVTASEWDYWGLGTLV |
| | PCT Publication No. | TVSS (SEQ ID NO: 77) |
| | WO 2011/073180 | |
| OX40 | Humanized nanobody | EVQLVESGGGLVQPGGSLRLSCAASGRTFSSIYAKGWFRQ |
| | OX40L026: | APGKEREFVAAISRSGRSTSYADSVKGRFTISRDNAKNTV |
| | SEQ ID NO: 202 of | YLQMNSLRPEDTAVYYCAAVGGATTVTASEWDYWGQGTLV |
| | PCT Publication No. | TVSS (SEQ ID NO: 78) |
| | WO 2011/073180 | |
| OX40 | Humanized nanobody | EVQLVESGGGLVQPGGSLRLSCAASGRTFSSIYAKGWFRQ |
| | OX40L027: | APGKEREFVAAISRSGRSTSYADSVKGRFTISRDNSKNTV |
| | SEQ ID NO: 203 of | YLQMNSLRPEDTAVYYCAAVGGATTVTASEWDYWGQGTLV |
| | PCT Publication No. | TVSS (SEQ ID NO: 79) |
| | WO 2011/073180 | |
| OX40 | Humanized nanobody | DVQLVESGGGLVQPGGSLRLSCAASGRTFSSIYAKGWFRQ |
| | OX40L028: | APGKEREFVAAISRSGRSTSYADSVKGRFTISRDNAKNTV |
| | SEQ ID NO: 204 of | YLQMNSLRPEDTAVYYCAAVGGATTVTASEWDYWGLGTLV |
| | PCT Publication No. | TVSS (SEQ ID NO: 80) |
| | WO 2011/073180 | |
| OX40 | Humanized nanobody | DVQLVESGGGLVQPGGSLRLSCAASGRTFSSIYAKGWFRQ |
| | OX40L039: | APGKEREFVAAISRSGRSTSYADSVKGRFTISRDNSKNTV |
| | SEQ ID NO: 205 of | YLQMNSLRPEDTAVYYCAAVGGATTVTASEWDYWGQGTLV |
| | PCT Publication No. | TVSS (SEQ ID NO: 81) |
| | WO 2011/073180 | |
| OX40 | Humanized nanobody | DVQLVESGGGLVQAGGSLRLSCAASRSIGRLDRMGWYRHR |
| | OX40L030: | TGEPRELVATITGGSSINYGDFVKGRFTISIDNAKNTVYL |
| | SEQ ID NO: 206 of | QMNNLKPEDTAVYYCNFNKYVTSRDTWGQGTQVTVSS |
| | PCT Publication No. | (SEQ ID NO: 82) |
| | WO 2011/073180 | |
| OX40 | Humanized nanobody | DVQLVESGGGLVQPGGSLRLSCAASRSIGRLDRMGWYRHR |
| | OX40L040: | TGEPRELVATITGGSSINYGDFVKGRFTISRDNSKNTVYL |
| | SEQ ID NO: 207 of | QMNSLRPEDTAVYYCNFNKYVTSRDTWGQGTLVTVSS |
| | PCT Publication No. | (SEQ ID NO: 83) |
| | WO 2011/073180 | |
| OX40 | Humanized nanobody | DVQLVESGGGLVQPGGSLRLSCAASRSIGRLDRMGWYRHA |
| | OX40L041: | TGEPRELVATITGGSSINYGDFVKGRFTISRDNSKNTVYL |
| | SEQ ID NO: 208 of | QMNSLRPEDTAVYYCNFNKYVTSRDTWGQGTLVTVSS |
| | PCT Publication No. | (SEQ ID NO: 84) |
| | WO 2011/073180 | |
| OX40 | Humanized nanobody | DVQLVESGGGLVQPGGSLRLSCAASRSIGRLDRMGWYRHR |
| | OX40L042: | PGEPRELVATITGGSSINYGDFVKGRFTISRDNSKNTVYL |
| | SEQ ID NO: 209 of | QMNSLRPEDTAVYYCNFNKYVTSRDTWGQGTLVTVSS |
| | PCT Publication No. | (SEQ ID NO: 85) |
| | WO 2011/073180 | |
| OX40 | Humanized nanobody | DVQLVESGGGLVQPGGSLRLSCAASRSIGRLDRMGWYRHR |
| | OX40L043: | TGKPRELVATITGGSSINYGDFVKGRFTISRDNSKNTVYL |
| | SEQ ID NO: 210 of | QMNSLRPEDTAVYYCNFNKYVTSRDTWGQGTLVTVSS |
| | PCT Publication No. | (SEQ ID NO: 86) |
| | WO 2011/073180 | |
| OX40 | Humanized nanobody | DVQLVESGGGLVQPGGSLRLSCAASRSIGRLDRMGWYRHA |
| | OX40L044: | PGEPRELVATITGGSSINYGDFVKGRFTISRDNSKNTVYL |
| | SEQ ID NO: 211 of | QMNSLRPEDTAVYYCNFNKYVTSRDTWGQGTLVTVSS |
| | PCT Publication No. | (SEQ ID NO: 87) |
| | WO 2011/073180 | |
| OX40 | Humanized nanobody | DVQLVESGGGLVQPGGSLRLSCAASRSIGRLDRMGWYRHA |
| | OX40L045: | TGKPRELVATITGGSSINYGDFVKGRFTISRDNSKNTVYL |
| | SEQ ID NO: 212 of | QMNSLRPEDTAVYYCNFNKYVTSRDTWGQGTLVTVSS |
| | PCT Publication No. | (SEQ ID NO: 88) |
| | WO 2011/073180 | |
| OX40 | Humanized nanobody | DVQLVESGGGLVQPGGSLRLSCAASRSIGRLDRMGWYRHR |
| | OX40L046: | PGKPRELVATITGGSSINYGDFVKGRFTISRDNSKNTVYL |
| | SEQ ID NO: 213 of | QMNSLRPEDTAVYYCNFNKYVTSRDTWGQGTLVTVSS |
| | PCT Publication No. | (SEQ ID NO: 89) |
| | WO 2011/073180 | |
| OX40 | Humanized nanobody | DVQLVESGGGLVQPGGSLRLSCAASRSIGRLDRMGWYRHA |
| | OX40L047: | PGKPRELVATITGGSSINYGDFVKGRFTISRDNSKNTVYL |
| | SEQ ID NO: 214 of | QMNSLRPEDTAVYYCNFNKYVTSRDTWGQGTLVTVSS |
| | PCT Publication No. | (SEQ ID NO: 90) |
| | WO 2011/073180 | |
| OX40 | Humanized nanobody | DVQLVESGGGLVQPGGSLRLSCAASRSIGRLDRMGWYRHR |
| | OX40L048: | TGEPRELVATITGGSSINYADFVKGRFTISRDNSKNTVYL |
| | SEQ ID NO: 215 of | QMNSLRPEDTAVYYCNFNKYVTSRDTWGQGTLVTVSS |
| | PCT Publication No. | (SEQ ID NO: 91) |
| | WO 2011/073180 | |
| OX40 | Humanized nanobody | DVQLVESGGGLVQPGGSLRLSCAASRSIGRLDRMGWYRHR |
| | OX40L049: | TGEPRELVATITGGSSINYGDSVKGRFTISRDNSKNTVYL |
| | SEQ ID NO: 216 of | QMNSLRPEDTAVYYCNFNKYVTSRDTWGQGTLVTVSS |
| | PCT Publication No. | (SEQ ID NO: 92) |
| | WO 2011/073180 | |
| OX40 | Humanized nanobody | DVQLVESGGGLVQPGGSLRLSCAASRSIGRLDRMGWYRHR |
| | OX40L050: | TGEPRELVATITGGSSINYADSVKGRFTISRDNSKNTVYL |
| | SEQ ID NO: 217 of | QMNSLRPEDTAVYYCNFNKYVTSRDTWGQGTLVTVSS |
| | PCT Publication No. | (SEQ ID NO: 93) |
| | WO 2011/073180 | |
| OX40 | Humanized nanobody | DVQLVESGGGLVQPGGSLRLSCAASRSIGRLDRMGWYRHR |
| | OX40L053: | TGEPRELVATITGGSSINYGDFVKGRFTISIDNSKNTVYL |
| | SEQ ID NO: 218 of | QMNSLRPEDTAVYYCNFNKYVTSRDTWGQGTLVTVSS |
| | PCT Publication No. | (SEQ ID NO: 94) |
| | WO 2011/073180 | |
| OX40 | Humanized nanobody | DVQLVESGGGLVQPGGSLRLSCAASRSIGRLDRMGWYRHR |
| | OX40L054: | TGEPRELVATITGGSSINYGDFVKGRFTISRDNAKNTVYL |
| | SEQ ID NO: 219 of | QMNSLRPEDTAVYYCNFNKYVTSRDTWGQGTLVTVSS |
| | PCT Publication No. | (SEQ ID NO: 95) |
| | WO 2011/073180 | |
| OX40 | Humanized nanobody | DVQLVESGGGLVQPGGSLRLSCAASRSIGRLDRMGWYRHR |
| | OX40L055: | TGEPRELVATITGGSSINYGDFVKGRFTISRDNSKNTVYL |
| | SEQ ID NO: 220 of | QMNNLRPEDTAVYYCNFNKYVTSRDTWGQGTLVTVSS |
| | PCT Publication No. | (SEQ ID NO: 96) |
| | WO 2011/073180 | |
| OX40 | Humanized nanobody | DVQLVESGGGLVQPGGSLRLSCAASRSIGRLDRMGWYRHR |
| | OX40L056: | PGKPRELVATITGGSSINYADSVKGRFTISRDNSKNTVYL |
| | SEQ ID NO: 221 of | QMNSLRPEDTAVYYCNFNKYVTSRDTWGQGTLVTVSS |
| | PCT Publication No. | (SEQ ID NO: 97) |
| | WO 2011/073180 | |
| OX40 | Humanized nanobody | DVQLVESGGGLVQPGGSLRLSCAASRSIGRLDRMGWYRHR |
| | OX40L069: | PGKPRELVATITGGSSINYADSVKGRFTISIDNSKNTVYL |
| | SEQ ID NO: 222 of | QMNSLRPEDTAVYYCNFNKYVTSRDTWGQGTLVTVSS |
| | PCT Publication No. | (SEQ ID NO: 98) |
| | WO 2011/073180 | |
| OX40 | Humanized nanobody | DVQLVESGGGLVQPGGSLRLSCAASRSIGRLDRMGWYRHR |
| | OX40L070: | PGKPRELVATITGGSSINYADSVKGRFTISRDNSKNTVYL |
| | SEQ ID NO: 223 of | QMNNLRPEDTAVYYCNFNKYVTSRDTWGQGTLVTVSS |
| | PCT Publication No. | (SEQ ID NO: 99) |
| | WO 2011/073180 | |
| OX40 | Humanized nanobody | DVQLVESGGGLVQPGGSLRLSCAASRSIGRLDRMGWYRHR |
| | OX40L071: | PGKPRELVATITGGSSINYADSVKGRFTISIDNSKNTVYL |
| | SEQ ID NO: 224 of | QMNNLRPEDTAVYYCNFNKYVTSRDTWGQGTLVTVSS |
| | PCT Publication No. | (SEQ ID NO: 100) |
| | WO 2011/073180 | |
| OX40 | Humanized nanobody | EVQLVESGGGLVQPGGSLRLSCAASRSIGRLDRMGWYRHR |
| | OX40L082: | PGEPRELVATITGGSSINYGDSVKGRFTISIDNSKNTVYL |
| | SEQ ID NO: 225 of | QMNSLRPEDTAVYYCNFNKYVTSRDTWGQGTLVTVSS |
| | PCT Publication No. | (SEQ ID NO: 101) |
| | WO 2011/073180 | |
| OX40 | Humanized nanobody | EVQLVESGGGLVQPGGSLRLSCAASRSIGRLDRMGWYRHR |
| | OX40L083: | PGKPRELVATITGGSSINYGDSVKGRFTISIDNSKNTVYL |
| | SEQ ID NO: 226 of | QMNSLRPEDTAVYYCNFNKYVTSRDTWGQGTLVTVSS |
| | PCT Publication No. | (SEQ ID NO: 102) |
| | WO 2011/073180 | |
| GITR | B09 sdAb: | QVQLQESGGXLVQSGGSLRLSCAASGSVFSIDAMGWYRLA |
| | SEQ ID NO: 19 of U.S. | PGKQRELVAVMSSGSPKYADSVKGRFTISRGSARGTVYLQ |
| | Pat. No. 10,093,742 | MDSLKPEDTAVYYCYADVATGWGRDASAYWGQGTQVTVSS |
| | | (SEQ ID NO: 103) |
| GITR | H09 sdAb: | QVQLQQSGGGLVRAGGSLRLSCVAAGSTFSVNSMAWYRQA |
| | SEQ ID NO: 20 of U.S. | PGKERELVAAFTGGSTMNYASSVKGRFTISRGNAAHTVLL |
| | Pat. No. 10,093,742 | QMTNLKPEDTAVYYCNAEVNEGWNADYHDYWGQGTQVTVS |
| | | S (SEQ ID NO: 104) |
| GITR | F05 sdAb: | QVQLVQSGGGLVQAGGSLRLSCTASGSIFSINHMAWYRQA |
| | SEQ ID NO: 21 of U.S. | PGKQREMVAHITGGASTKYADSVKGRFTISRDSALNTVSL |
| | Pat. No. 10,093,742 | RMNSLKPEDTAVYYCNAEVNEGWNADYYDVWGQGTQVTVS |
| | | S (SEQ ID NO: 105) |
| GITR | C06 sdAb: | QVQLQESGGGLVQAGGSLRLSCAASGSVFSIDAMGWYRLA |
| | SEQ ID NO: 22 of U.S. | PGQQRELVAVLNGISSAKYADSVKGRFTISGDSAKNAVYL |
| | Pat. No. 10,093,742 | QMDGLKPEDTAVYYCYADVSTGWGRDAHGYWGQGTQVTVS |
| | | S (SEQ ID NO: 106) |
| GITR | 2A1 sdAb: | EVQLVQSGGGLVQPGGSLRLSCAASGNIFSIDAMGWYRQA |
| | SEQ ID NO: 23 of U.S. | PGRQRELVAQIPGGPTDSVKGRFTVSGNSAKNTGYLQMNT |
| | Pat. No. 10,093,742 | LKPEDTAVYYCNIVASTSWGSPSKVYWGQGTQATVSS |
| | | (SEQ ID NO: 107) |
| GITR | E2 sdAb: | QVQLQESGGGLVQPGGSLRLSCAASGSVFSIDSMSWFRQA |
| | SEQ ID NO: 24 of U.S. | PGNERELVALITGGRTTTYADSVKGRFTISRASAPNTVYL |
| | Pat. No. 10,093,742 | QMNSLKPEDTAVYYCNAVVSTGWGRNADDYWGQGTQVTVS |
| | | (SEQ ID NO: 108) |
| GITR | B12 sdAb: | QVQLQQSGGGLVQAGGSLRLSCAASGSIFSIDAMGWYRLA |
| | SEQ ID NO: 25 of U.S. | PGKQRELVAVIDGVSPNYADSVKGRFTISSDIAKNTVYLQ |
| | Pat. No. 10,093,742 | MHSPKPEDTAVYYCNADVSTGWGRPADHYWGQGTQVTVS |
| | | (SEQ ID NO: 109) |
| GITR | B2 sdAb: | QVQLQESGGGLVQPGGSLRLSCAASGSVFSIDSMSWFRQA |
| | SEQ ID NO: 26 of U.S. | PGNERELVALITGGHTTTYGDSVKGRFTISRASAPNTVHL |
| | Pat. No. 10,093,742 | QMNSLQPEDTAVYYCNAAVSTGWGRNADDYWGQGTQVTVS |
| | | (SEQ ID NO: 110) |
| GITR | F2 sdAb: | QLVQSGGGLVQPGESLRLSCAASGSVFSIDSVSWFRQGPG |
| | SEQ ID NO: 27 of U.S. | NERELVALITGGRTTTYADSVKGRFTISRANAPNTVHLRM |
| | Pat. No. 10,093,742 | NSLKPEDTAVYYCNAAVSTGWGRNADDYWGQGTQVTVS |
| | | (SEQ ID NO: 111) |
| GITR | B3 sdAb: | QVQLVQSGGGLVQPGGSLRLICAASGSVFSIDSMSWFRQR |
| | SEQ ID NO: 28 of U.S. | PGNERELVALITGGRTTTYSDSVKGRFTISRASALNTVHL |
| | Pat. No. 10,093,742 | QMNSLKPEDTAVYYCNAALSTGWGRDASAYWGQGTQVTVS |
| | | (SEQ ID NO: 112) |
| GITR | E3 sdAb: | QVQLQESGGGLVQAGGSLRLSCTASGSIFSINHMAWYRQA |
| | SEQ ID NO: 29 of U.S. | PGKQREMVAHITGGASTKYADSVKGRFTISRDSALNTVSL |
| | Pat. No. 10,093,742 | RMNSLKPEDTAVYYCNAEVNEGWNADYYDVWGQGTQVTVS |
| | | (SEQ ID NO: 113) |
| GITR | B4 sdAb: | QLQLQESGGGTVQAGGSLRLSCAASRSIASINVMGWYRQA |
| | SEQ ID NO: 30 of U.S. | PGNQHELVAAITSGGSPNYAGSVRGRFIISRDNAKNTVYL |
| | Pat. No. 10,093,742 | QMNDLKPEDTAVYYCAGELRDDSNGYLHYWGQGTQVTVS |
| | | (SEQ ID NO: 114) |
| GITR | B7 sdAb: | QVQLQESGGGLVQPGGSLRLSCAASGSVFSIDSMSWFRQT |
| | SEQ ID NO: 31 of U.S. | PGNERELVAHITGGRTTTYADSVKGRFTISRASAPNTVHL |
| | Pat. No. 10,093,742 | QMNNLKPEDTAVYYCNAAVSTGWGRNADDYWGQGTQVTVS |
| | | (SEQ ID NO: 115) |
| GITR | C7 sdAb: | QVQLQESGGGLVQAGGSLRLSCTASGSIFSIDDMGWYRLA |
| | SEQ ID NO: 32 of U.S. | PGKQRELVAVHSGSSTNYGDSVKGRFTISGDSAKNTVYLQ |
| | Pat. No. 10,093,742 | MHRLEPEDTAVYYCYAAISSGWGRDAEDYWGQGTQVTVS |
| | | (SEQ ID NO: 116) |
| GITR | C4 sdAb: | QVQLVQSGGGLVQPGESLRLSCAASGSVFSIDSMSWFRQG |
| | SEQ ID NO: 33 of U.S. | PGNERELVALITGGRTTTYADSVKGRFTISRANAPNTVHL |
| | Pat. No. 10,093,742 | QMNSLKPEDTAVYYCNAAVSTGWGRSADDYWGQGTQVTVS |
| | | (SEQ ID NO: 117) |
| GITR | B5 sdAb: | QVQLVQSGGGLVQPGESLRLSCAASGSVFSIDSMSWFRQG |
| | SEQ ID NO: 34 of U.S. | PGNERELVALITGGRTTTYADSVKGRFTISRANAPNTVHL |
| | Pat. No. 10,093,742 | QMNSLEPEDTAVYYCNAAVSTGWGRNADDYWGQGTQVTVS |
| | | (SEQ ID NO: 118) |
| GITR | H11 sdAb: | QVQLVQSGGGLVQPGGSLRLSCAASGSVFSIDSMSWFRQA |
| | SEQ ID NO: 35 of U.S. | PGNERELVALITGGRTTTYADSVKGRFTISRASAPNTVHL |
| | Pat. No. 10,093,742 | QMNSLKPEDTAVYYCNAVVSTGWGRNADDYWGQGTQVTVS |
| | | (SEQ ID NO: 119) |
| GITR | H11v420 sdAb: | EVQLLESGGGEVQPGGSLRLSCAASGSVFSIDAMSWFRQA |
| | SEQ ID NO: 36 of U.S. | PGKGLELVSAITGGRTTYYAESVKGRFTISRDNAKNTLYL |
| | Pat. No. 10,093,742 | QMSSLRAEDTAVYYCNAVVSTGWGRNADDYWGQGTLVTVK |
| | | P (SEQ ID NO: 120) |
| GITR | H11v420.1 sdAb: | EVQLLESGGGEVQPGGSLRLSCAASGSVFSIDAMSWFRQA |
| | SEQ ID NO: 37 of U.S. | PGKGLELVCAITGGRTTYYAESVKGRFTCSRDNAKNTLYL |
| | Pat. No. 10,093,742 | QMSSLRAEDTAVYYCNAVVSTGWGRNADDYWGQGTLVTVK |
| | | P (SEQ ID NO: 121) |
| GITR | H11v401 sdAb: | EVQLLESGGGEVQPGGSLRLSCAASGSVFSIDSMSWFRQA |
| | SEQ ID NO: 38 of U.S. | PGKGLELVSLITGGRTTYYAESVKGRFTISRDNAKNTLYL |
| | Pat. No. 10,093,742 | QMSSLRAEDTAVYYCNAVVSTGWGRNADDYWGQGTLVTVK |
| | | P (SEQ ID NO: 122) |
| GITR | H11v401.1 sdAb: | EVQLLESGGGEVQPGGSLRLSCAASGSVFSIDSMSWFRQA |
| | SEQ ID NO: 39 of U.S. | PGKGLELVCLITGGRTTYYAESVKGRFTCSRDNAKNTLYL |
| | Pat. No. 10,093,742 | QMSSLRAEDTAVYYCNAVVSTGWGRNADDYWGQGTLVTVK |
| | | P (SEQ ID NO: 123) |
| GITR | H11v421 sdAb: | EVQLLESGGGEVQPGGSLRLSCAASGSVFSIDAMSWFRQA |
| | SEQ ID NO: 40 of U.S. | PGKGLELVSLITGGRTTYYAESVKGRFTISRDNAKNTLYL |
| | Pat. No. 10,093,742 | QMSSLRAEDTAVYYCNAVVSTGWGRNADDYWGQGTLVTVK |
| | | P (SEQ ID NO: 124) |
| GITR | H11v421.1 sdAb: | EVQLLESGGGEVQPGGSLRLSCAASGSVFSIDAMSWFRQA |
| | SEQ ID NO: 41 of U.S. | PGKGLELVCLITGGRTTYYAESVKGRFTCSRDNAKNTLYL |
| | Pat. No. 10,093,742 | QMSSLRAEDTAVYYCNAVVSTGWGRNADDYWGQGTLVTVK |
| | | P (SEQ ID NO: 125) |
| GITR | hzC06v1.1 sdAb: | EVQLLESGGGEVQPGGSLRLSCAASGSVFSIDAMGWYRQA |
| | SEQ ID NO: 42 of U.S. | PGKGLELVSALSGISSATYAESVKGRFTISRDNAKNTLYL |
| | Pat. No. 10,093,742 | QMSSLRAEDTAVYYCYADVSTGWGRDAHGYWGQGTLVTV |
| | | (SEQ ID NO: 126) |
| GITR | hzC06v1.2 sdAb: | EVQLLESGGGEVQPGGSLRLSCAASGSVFSIDAMGWYRQA |
| | SEQ ID NO: 43 of U.S. | PGKGRELVSALSGISSATYAESVKGRFTISRDNAKNTLYL |
| | Pat. No. 10,093,742 | QMSSLRAEDTAVYYCYADVSTGWGRDAHGYWGQGTLVTV |
| | | (SEQ ID NO: 127) |
| GITR | hzC06v1.3 sdAb: | EVQLLESGGGEVQPGGSLRLSCAASGSVFSIDAMGWYRQA |
| | SEQ ID NO: 44 of U.S. | PGKQRELVSALSGISSATYAESVKGRFTISRDNAKNTLYL |
| | Pat. No. 10,093,742 | QMSSLRAEDTAVYYCYADVSTGWGRDAHGYWGQGTLVTV |
| | | (SEQ ID NO: 128) |
| GITR | hzC06v1.4 sdAb: | EVQLLESGGGEVQPGGSLRLSCAASGSVFSIDAMGWYRQA |
| | SEQ ID NO: 45 of U.S. | PGQQRELVSALSGISSATYAESVKGRFTISRDNAKNTLYL |
| | Pat. No. 10,093,742 | QMSSLRAEDTAVYYCYADVSTGWGRDAHGYWGQGTLVTV |
| | | (SEQ ID NO: 129) |
| GITR | hzC06v2.1 sdAb: | EVQLLESGGGEVQPGGSLRLSCAASGSVFSIDAMGWYRQA |
| | SEQ ID NO: 46 of U.S. | PGKGLELVAVLSGISSATYAESVKGRFTISRDNAKNTLYL |
| | Pat. No. 10,093,742 | QMSSLRAEDTAVYYCYADVSTGWGRDAHGYWGQGTLVTV |
| | | (SEQ ID NO: 130) |
| GITR | hzC06v2.2 sdAb: | EVQLLESGGGEVQPGGSLRLSCAASGSVFSIDAMGWYRQA |
| | SEQ ID NO: 47 of U.S. | PGKGRELVAVLSGISSATYAESVKGRFTISRDNAKNTLYL |
| | Pat. No. 10,093,742 | QMSSLRAEDTAVYYCYADVSTGWGRDAHGYWGQGTLVTV |
| | | (SEQ ID NO: 131) |
| GITR | hzC06v2.3 sdAb: | EVQLLESGGGEVQPGGSLRLSCAASGSVFSIDAMGWYRQA |
| | SEQ ID NO: 48 of U.S. | PGKQRELVAVLSGISSATYAESVKGRFTISRDNAKNTLYL |
| | Pat. No. 10,093,742 | QMSSLRAEDTAVYYCYADVSTGWGRDAHGYWGQGTLVTV |
| | | (SEQ ID NO: 132) |
| GITR | hzC06v2.4 sdAb: | EVQLLESGGGEVQPGGSLRLSCAASGSVFSIDAMGWYRQA |
| | SEQ ID NO: 49 of U.S. | PGQQRELVAVLSGISSATYAESVKGRFTISRDNAKNTLYL |
| | Pat. No. 10,093,742 | QMSSLRAEDTAVYYCYADVSTGWGRDAHGYWGQGTLVTV |
| | | (SEQ ID NO: 133) |
| GITR | hzC06v3 sdAb: | EVQLLESGGGEVQPGGSLRLSCAASGSVFSIDAMGWYRQA |
| | SEQ ID NO: 50 of U.S. | PGKQRELVAVLSGISSAKYAESVKGRFTISRDNAKNTLYL |
| | Pat. No. 10,093,742 | QMSSLRAEDTAVYYCYADVSTGWGRDAHGYWGQGTLVTV |
| | | (SEQ ID NO: 134) |
| GITR | hzC06v3.1 sdAb: | EVQLLESGGGEVQPGGSLRLSCAASGSVFSIDAMGWYRLA |
| | SEQ ID NO: 51 of U.S. | PGQQRELVAVLSGISSAKYAESVKGRFTISRDNAKNTLYL |
| | Pat. No. 10,093,742 | QMSSLRAEDTAVYYCYADVSTGWGRDAHGYWGQGTLVTV |
| | | (SEQ ID NO: 135) |
| GITR | hzC06v3.2 sdAb: | EVQLLESGGGEVQPGGSLRLSCAASGSVFSIDAMGWYRQA |
| | SEQ ID NO: 52 of U.S. | PGKQRELVAVLSGISSAKYADSVKGRFTISGDNAKNTLYL |
| | Pat. No. 10,093,742 | QMSSLRAEDTAVYYCYADVSTGWGRDAHGYWGQGTLVTV |
| | | (SEQ ID NO: 136) |
| GITR | hzC06v3.3 sdAb: | EVQLLESGGGEVQPGGSLRLSCAASGSVFSIDAMGWYRQA |
| | SEQ ID NO: 53 of U.S. | PGKQRELVAVLSGISSAKYAESVKGRFTISRDSAKNAVYL |
| | Pat. No. 10,093,742 | QMDGLKPEDTAVYYCYADVSTGWGRDAHGYWGQGTLVTV |
| | | (SEQ ID NO: 137) |
| GITR | hzC06v3.4 sdAb: | EVQLLESGGGEVQPGGSLRLSCAASGSVFSIDAMGWYRQA |
| | SEQ ID NO: 54 of U.S. | PGKQRELVAVLSGISSAKYAESVKGRFTISRDNAKNTVYL |
| | Pat. No. 10,093,742 | QMSSLRAEDTAVYYCYADVSTGWGRDAHGYWGQGTLVTV |
| | | (SEQ ID NO: 138) |
| GITR | hzC06v3.5 sdAb: | EVQLLESGGGEVQPGGSLRLSCAASGSVFSIDAMGWYRQA |
| | SEQ ID NO: 55 of U.S. | PGKQRELVAVLSGISSAKYAESVKGRFTISRASAPNTLYL |
| | Pat. No. 10,093,742 | QMSSLRAEDTAVYYCYADVSTGWGRDAHGYWGQGTLVTV |
| | | (SEQ ID NO: 139) |
| GITR | hzC06v3.6 sdAb: | EVQLLESGGGEVQPGGSLRLSCAASGSVFSIDAMGWYRQA |
| | SEQ ID NO: 56 of U.S. | PGKQRELVAVLSGISSAKYAESVKGRFTISRASAPNTVYL |
| | Pat. No. 10,093,742 | QMSSLRAEDTAVYYCYADVSTGWGRDAHGYWGQGTLVTV |
| | | (SEQ ID NO: 140) |
| GITR | hzC06v3.7 sdAb: | EVQLLESGGGEVQPGGSLRLSCAASGSVFSIDAMGWYRQA |
| | SEQ ID NO: 57 of U.S. | PGKQRELVAVLSGISSAKYAASAPGRFTISRDAVKNTVYL |
| | Pat. No. 10,093,742 | QMSSLRAEDTAVYYCYADVSTGWGRDAHGYWGQGTLVTV |
| | | (SEQ ID NO: 141) |
| GITR | hzC06v3.8 sdAb: | EVQLLESGGGEVQPGGSLRLSCAASGSVFSIDAMGWYRQA |
| | SEQ ID NO: 58 of U.S. | PGKQRELVAVLSGISSAKYAASAPGRFTISRDAVENTVYL |
| | Pat. No. 10,093,742 | QMSSLRAEDTAVYYCYADVSTGWGRDAHGYWGQGTLVTV |
| | | (SEQ ID NO: 142) |
| GITR | hzC06v3.9 sdAb: | EVQLLESGGGEVQPGGSLRLSCAASGSVFSIDAMGWYRQA |
| | SEQ ID NO: 59 of U.S. | PGKQRELVAVLSGISSAKYAASAPGRFTISRDNAKNTVYL |
| | Pat. No. 10,093,742 | QMSSLRAEDTAVYYCYADVSTGWGRDAHGYWGQGTLVTV |
| | | (SEQ ID NO: 143) |
| GITR | hzC06v3.10 sdAb: | EVQLLESGGGEVQPGGSLRLSCAASGSVFSIDAMGWYRQA |
| | SEQ ID NO: 60 of U.S. | PGKQRELVAVLSGISSAKYADAVKGRFTISRASAPNTVYL |
| | Pat. No. 10,093,742 | QMSSLRAEDTAVYYCYADVSTGWGRDAHGYWGQGTLVTV |
| | | (SEQ ID NO: 144) |
| GITR | hzC06v3.11 sdAb: | EVQLLESGGGEVQPGGSLRLSCAASGSVFSIDAMGWYRQA |
| | SEQ ID NO: 61 of U.S. | PGKQRELVAVLSGISSAKYADAVEGRFTISRASAPNTVYL |
| | Pat. No. 10,093,742 | QMSSLRAEDTAVYYCYADVSTGWGRDAHGYWGQGTLVTV |
| | | (SEQ ID NO: 145) |
| GITR | hzC06v3.12 sdAb: | EVQLLESGGGEVQPGGSLRLSCAASGSVFSIDAMGWYRQA |
| | SEQ ID NO: 62 of U.S. | PGKQRELVAVLSGISSAKYAASAPGRFTISRASAPNTVYL |
| | Pat. No. 10,093,742 | QMSSLRAEDTAVYYCYADVSTGWGRDAHGYWGQGTLVTV |
| | | (SEQ ID NO: 146) |
| GITR | hzC04v1 sdAb: | EVQLLESGGGEVQPGGSLRLSCAASGFTFSTHGMDWFRQA |
| | SEQ ID NO: 63 of U.S. | PGKDLEWVSAINNGGSWTSYASSVKGRFTISRDNAKNTLY |
| | Pat. No. 10,093,742 | LQMSSLRAEDTAVYWCQNRVTRGQGTLVTV (SEQ ID |
| | | NO: 147) |
| GITR | hzC04v1.2 sdAb: | EVQLLESGGGEVQPGGSLRLSCAASGFTFSTHGMDWFRQA |
| | SEQ ID NO: 64 of U.S. | PGKDLEWVSAINNGGSWTSYASSVKGRFTISRDNAKNTLY |
| | Pat. No. 10,093,742 | LQMSSLRAEDTAVYYCQNRVTRGQGTLVTV (SEQ ID |
| | | NO: 148) |
| GITR | hzC04v1.2.1 sdAb: | EVQLLESGGGEVQPGGSLRLSCAASGFTFSTHGMDWFRQA |
| | SEQ ID NO: 65 of U.S. | PGKDLEWVSAINQGGSWTSYASSVKGRFTISRDNAKNTLY |
| | Pat. No. 10,093,742 | LQMSSLRAEDTAVYYCQNRVTRGQGTLVTV (SEQ ID |
| | | NO: 149) |
| GITR | hzC04v2 sdAb: | EVQLLESGGGEVQPGGSLRLSCAASGFTFSTHGMDWFRQA |
| | SEQ ID NO: 66 of U.S. | PGKGLEWVSAINNGGSWTSYASSVKGRFTISRDNAKNTLY |
| | Pat. No. 10,093,742 | LQMSSLRAEDTAVYWCQNRVTRGQGTLVTV (SEQ ID |
| | | NO: 150) |
| GITR | hzC04v2.2 sdAb: | EVQLLESGGGEVQPGGSLRLSCAASGFTFSTHGMDWFRQA |
| | SEQ ID NO: 67 of U.S. | PGKGLEWVSAINNGGSWTSYASSVKGRFTISRDNAKNTLY |
| | Pat. No. 10,093,742 | LQMSSLRAEDTAVYYCQNRVTRGQGTLVTV (SEQ ID |
| | | NO: 151) |
| GITR | hzC04v5 sdAb: | EVQLLESGGGEVQPGGSLRLSCAASGFTFSTHGMDWFRQA |
| | SEQ ID NO: 68 of U.S. | PGKDLEWVSAIQSGGSWTSYASSVKGRFTISRDNAKNTLY |
| | Pat. No. 10,093,742 | LQMSSLRAEDTAVYWCQNRVTRGQGTLVTV (SEQ ID |
| | | NO: 152) |
| GITR | hzC04v5.1 sdAb: | EVQLLESGGGEVQPGGSLRLSCAASGFTFSTHGMDWFRQA |
| | SEQ ID NO: 69 of U.S. | PGKDLEWVSAIQSGGSWTSYASSVKGRFTISRDNAKNTLY |
| | Pat. No. 10,093,742 | LEMNNLKPEDTAVYWCQNRVTRGQGTLVTV (SEQ ID |
| | | NO: 153) |
| GITR | hzC04v5.2 sdAb: | EVQLLESGGGEVQPGGSLRLSCAASGFTFSTHGMDWFRQA |
| | SEQ ID NO: 70 of U.S. | PGKDLEWVSAIQSGGSWTSYASSVKGRFTISRDNAKNTLY |
| | Pat. No. 10,093,742 | LQMNNLRAEDTAVYWCQNRVTRGQGTLVTV (SEQ ID |
| | | NO: 154) |
| GITR | hzC04v5.3 sdAb: | EVQLLESGGGEVQPGGSLRLSCAASGFTFSTHGMDWFRQA |
| | SEQ ID NO: 71 of U.S. | PGKDLEWVSAIQSGGSWTSYASSVKGRFTISRDNAKNTLY |
| | Pat. No. 10,093,742 | LEMSSLRAEDTAVYWCQNRVTRGQGTLVTV (SEQ ID |
| | | NO: 155) |
| GITR | hzC04v5.4 sdAb: | EVQLLESGGGEVQPGGSLRLSCAASGFTFSTHGMDWFRQA |
| | SEQ ID NO: 72 of U.S. | PGKDLEWVSAIQSGGSWTSYASSVKGRFTISRDNAKNTLY |
| | Pat. No. 10,093,742 | LQMSSLRPEDTAVYWCQNRVTRGQGTLVTV (SEQ ID |
| | | NO: 156) |
| GITR | hzC04v5.5 sdAb: | EVQLLESGGGEVQPGGSLRLSCAASGFTFSTHGMDWFRQA |
| | SEQ ID NO: 73 of U.S. | PGKDLEWVSAIQSGGSWTSYASSVKGRFTISRDNAKNTLY |
| | Pat. No. 10,093,742 | LQMQQLRAEDTAVYWCQNRVTRGQGTLVTV (SEQ ID |
| | | NO: 157) |
| GITR | hzC04v5.6 sdAb: | EVQLLESGGGEVQPGGSLRLSCAASGFTFSTHGMDWFRQA |
| | SEQ ID NO: 74 of U.S. | PGKDLEWVSAIQSGGSWTSYASSVKGRFTISRDNAKNTLY |
| | Pat. No. 10,093,742 | LQMQNLRAEDTAVYWCQNRVTRGQGTLVTV (SEQ ID |
| | | NO: 158) |
| GITR | hzC04v5.7 sdAb: | EVQLLESGGGEVQPGGSLRLSCAASGFTFSTHGMDWFRQA |
| | SEQ ID NO: 75 of U.S. | PGKDLEWVSAIQSGGSWTSYASSVKGRFTISRDNAKNTLY |
| | Pat. No. 10,093,742 | LQMDNLRAEDTAVYWCQNRVTRGQGTLVTV (SEQ ID |
| | | NO: 159) |
| GITR | hzC04v5.8 sdAb: | EVQLLESGGGEVQPGGSLRLSCAASGFTFSTHGMDWFRQA |
| | SEQ ID NO: 76 of U.S. | PGKDLEWVSAIQSGGSWTSYASSVKGRFTISRDNAKNTLY |
| | Pat. No. 10,093,742 | LQMNDLRAEDTAVYWCQNRVTRGQGTLVTV (SEQ ID |
| | | NO: 160) |
| GITR | hzC04v5.9 sdAb: | EVQLLESGGGEVQPGGSLRLSCAASGFTFSTHGMDWFRQA |
| | SEQ ID NO: 77 of U.S. | PGKDLEWVSAIQSGGSWTSYASSVKGRFTISRDNAKNTLY |
| | Pat. No. 10,093,742 | LQMDDLRAEDTAVYWCQNRVTRGQGTLVTV (SEQ ID |
| | | NO: 161) |
| GITR | hzC04v5.10 sdAb: | EVQLLESGGGEVQPGGSLRLSCAASGFTFSTHGMDWFRQA |
| | SEQ ID NO: 78 of U.S. | PGKDLEWVSAIQSGGSWTSYASSVKGRFTISRDNAKNTLY |
| | Pat. No. 10,093,742 | LQMQDLRAEDTAVYWCQNRVTRGQGTLVTV (SEQ ID |
| | | NO: 162) |
| GITR | hzC04v5.11 sdAb: | EVQLLESGGGEVQPGGSLRLSCAASGFTFSTHGMDWFRQA |
| | SEQ ID NO: 79 of U.S. | PGKDLEWVSAIQSGGSWTSYASSVKGRFTISRDNAKNTLY |
| | Pat. No. 10,093,742 | LQMNQLRAEDTAVYWCQNRVTRGQGTLVTV (SEQ ID |
| | | NO: 163) |
| GITR | hzC04v5.12 sdAb: | EVQLLESGGGEVQPGGSLRLSCAASGFTFSTHGMDWFRQA |
| | SEQ ID NO: 80 of U.S. | PGKDLEWVSAIQSGGSWTSYASSVKGRFTISRDNAKNTLY |
| | Pat. No. 10,093,742 | LQMSNLRAEDTAVYWCQNRVTRGQGTLVTV (SEQ ID |
| | | NO: 164) |
| CD40 | V19 VHH: | QVQLQESGGGLVQAGGSLRLSCAASGRTFGRSAMGWFRQA |
| | SEQ ID NO: 13 of PCT | PGKEREFVAAIGTRGGSTKYADSVKGRFTISTDNASNTVY |
| | Publication No. WO | LQMDSLKPEDTAVYRCAVRGPGYPSAAIFQDEYHYWGQGT |
| | 2020/159368 A1 | QVTVSS (SEQ ID NO: 165) |
| CD40 | V19t VHH: | QVQLQESGGGLVQAGGSLRLSCAASGRTFGRSAMGWFRQA |
| | SEQ ID NO: 15 of PCT | PGKEREFVAAIGTRGGSTKYADSVKGRFTISTDNASNTVY |
| | Publication No. WO | LQMDSLKPEDTAVYRCAVRGPGYPSAAIFQDEYHYWGQGT |
| | 2020/159368 A1 | QVTVSSGLEGHSDHMEQKLISEEDLNRISDHHHHHH |
| | | (SEQ ID NO: 166) |
| CD40 | V15 VHH: | EVQLQESGGGLVQAGGSLRLSCVTSGSAFSSDTMGWFRQA |
| | SEQ ID NO: 14 of PCT | PGKQRELVASISSRGVREYADSVKGRFTISRDNAKNTVYL |
| | Publication No. WO | QMNSLQPEDTAVYYCNRGALGLPGYRPYNNWGQGTQVTVS |
| | 2020/159368 A1 | S (SEQ ID NO: 167) |
| CD40 | V15t VHH: | EVQLQESGGGLVQAGGSLRLSCVTSGSAFSSDTMGWFRQA |
| | SEQ ID NO: 16 of PCT | PGKQRELVASISSRGVREYADSVKGRFTISRDNAKNTVYL |
| | Publication No. WO | QMNSLQPEDTAVYYCNRGALGLPGYRPYNNWGQGTQVTVS |
| | 2020/159368 A1 | SGLEGHSDHMEQKLISEEDLNRISDHHHHHH (SEQ ID |
| | | NO: 168) |
| CD40 | V12 VHH: | QVQLQESGGGLVQAGGSLRLSCAASGLVFKRYSMNWYRQP |
| | SEQ ID NO: 35 of PCT | PGQQRGLVASISDSGVSTNYADSVKGRFTISRDNAKNIGY |
| | Publication No. WO | LQMNSLKPEDTAVYYCNMHTFWGQGTQVTVSS (SEQ ID |
| | 2020/159368 A1 | NO: 169) |
| CD40 | V12t VHH: | QVQLQESGGGLVQAGGSLRLSCAASGLVFKRYSMNWYRQP |
| | SEQ ID NO: 36 of PCT | PGQQRGLVASISDSGVSTNYADSVKGRFTISRDNAKNIGY |
| | Publication No. WO | LQMNSLKPEDTAVYYCNMHTFWGQGTQVTVSSGLEGHSDH |
| | 2020/159368 A1 | MEQKLISEEDLNRISDHHHHHH (SEQ ID NO: 170) |
| 4-1BB | 4H04 sdAb: | QVQLQESGGGLVQAGDSLRLSCAASGWAFDNYGMAWFRQA |
| | SEQ ID IN:16 of U.S. | PGKEREFIGRLAWNGGSTDYADSVKGRFTISRDNPKNTLY |
| | Publication No. | LQMNNLKPEDTAVYYCARQRSYSGYGIRTPQTYDYWGQGT |
| | 2017/0198050 A1 | QVT (SEQ ID NO: 171) |
| 4-1BB | 4E1 sdAb: | QVQLQQSGGGLVQAGDSLRLSCAASGWAFGNYGMAWFRRA |
| | SEQ ID IN:20 of U.S. | PGKEREFIGRLAWNGGSTDYVDSVKGRFTISRDNPKNTLY |
| | Publication No. | LQMNNLKPDDTAVYYCARQRSYSRYDIRTPQTYDYWGQGT |
| | 2017/0198050 A1 | QVT (SEQ ID NO: 172) |
| 4-1BB | 4F5 sdAb: | QVQLVQSGGGLVQPGGSLRLSCAASGWAFDNYGMAWFRQA |
| | SEQ ID IN:23 of U.S. | PGKEREFIGRLAWNGGSTDYADSVKGRFTISRDNPKNTLY |
| | Publication No. | LQMNSLKPEDTAVYYCARQRSYSRYGIRAPQTYDYWGQGT |
| | 2017/0198050 A1 | QVT (SEQ ID NO: 173) |
| 4-1BB | RH3 sdAb: | QVQLQESGGGLVQPGGSLRLSCAVSGFSFSINAMGWYRQA |
| | SEQ ID IN:25 of U.S. | PGKRREFLAAIDSGRNTVYAVSVKGRFTISRDNAKNTVYL |
| | Publication No. | QMNSLKPEDTAIYYCGLLKGNRVVSPSVAYWGQGTQVT |
| | 2017/0198050 A1 | (SEQ ID NO: 174) |
| 4-1BB | D1 sdAb: | EVQPVQSGGGLVQAGESLRLSCAASATIFSNNAMGWYRQA |
| | SEQ ID IN:29 of U.S. | PGKQRELVATITTGGFTNYRDSVKGRFDISRDNAKNTVYL |
| | Publication No. | QMNNLKPEDTAVYYCNVVLRYSRDYSYTTVKEYWGQGTQV |
| | 2017/0198050 A1 | (SEQ ID NO: 175) |
| 4-1BB | 1G3 sdAb: | QVQLVQSGGGLVQPGGSLRLSCAASGFTFSSYAMSWVRQA |
| | SEQ ID IN:432 of U.S. | PGKGLEWVSAIPAGDGSTKYADSVKGRFTISRDNAKNTVY |
| | Publication No. | LQMDSLKPEDTAVYFCAKSRGWSTVDDMDYWGKGTQV |
| | 2017/0198050 A1 | (SEQ ID NO: 176) |
| 4-1BB | 1H4 sdAb: | QVQLVQSGGGLVQPGGSLRLSCVVSGFTFRSYAMSWVRQA |
| | SEQ ID IN:436 of U.S. | PGKGLEWVSTINSGESSTKYADSVKGRFTISRDDAKNTLY |
| | Publication No. | LQMSDLKPEDTAVYFCAKHRGWSTVDDINYWGKGTQV |
| | 2017/0198050 A1 | (SEQ ID NO: 177) |
| 4-1BB | 1H1 sdAb: | QVQLVQSGGGLVQPGGSLRLSCAASGFTFDDHAMSWVRQA |
| | SEQ ID IN:440 of U.S. | PGKGLEWVSAISWNGHYTYYAESMKGRFAISRDNAKNTLY |
| | Publication No. | LQMNSLKSEDTAVYYCVKGWRGSYTRDRPFASWGQGTQV |
| | 2017/0198050 A1 | (SEQ ID NO: 178) |
| 4-1BB | 1H8 sdAb: | EVQLVQSGGGLVQPGGSLRLSCAASGFTFSSYYMSWVRQA |
| | SEQ ID IN:444 of U.S. | PGKGLEWVSTISTNTGGGSTYYAYADSVKGRFTISRDNAK |
| | Publication No. | NTLYLEMNSLKPEDTAQYYCVRTRWEGVYDYWGLGTQV |
| | 2017/0198050 A1 | (SEQ ID NO: 179) |
| 4-1BB | Hz4E1-v1 sdAb: | EVQLLESGGGEVQPGGSLRLSCAASGWAFGNYGMAWFRQA |
| | SEQ ID IN:33 of U.S. | PGKGLEWVARLAWNGGSTDYAESVKGRFTISRDNAKNTLY |
| | Publication No. | LQMSSLRAEDTAVYYCARQRSYSRYDIRTPQTYDYWGQGT |
| | 2017/0198050 A1 | LVTVKP (SEQ ID NO: 180) |
| 4-1BB | Hz4E1-v3 sdAb: | EVQLLESGGGEVQPGGSLRLSCAASGWAFGNYGMAWFRQA |
| | SEQ ID IN:34 of U.S. | PGKGREFVARLAWNGGSTDYAESVKGRFTISRDNAKNTLY |
| | Publication No. | LQMSSLRAEDTAVYYCARQRSYSRYDIRTPQTYDYWGQGT |
| | 2017/0198050 A1 | LVTVKP (SEQ ID NO: 181) |
| 4-1BB | Hz4E01v7-1 sdAb: | EVQLLESGGGEVQPGGSLRLSCAASGWAFGNYGMAWFRQA |
| | SEQ ID IN:35 of U.S. | PGKEREFVSRLAWNGGSTDYVAESVKGRFTISRDNAKNTL |
| | Publication No. | YLQMSSLRAEDTAVYYCARQRSYSRYDIRTPQTYDYWGQG |
| | 2017/0198050 A1 | TLVTVK (SEQ ID NO: 182) |
| 4-1BB | Hz4E01v8 sdAb: | EVQLLESGGGEVQPGGSLRLSCAASGWAFGNYGMAWFRQA |
| | SEQ ID IN:36 of U.S. | PGKEREFIGRLAWNGGSTDYVESVKGRFTISRDNPKNTLY |
| | Publication No. | LQMSSLRAEDTAVYYCARQRSYSRYDIRTPQTYDYWGQGT |
| | 2017/0198050 A1 | LVTVKP (SEQ ID NO: 183) |
| 4-1BB | Hz4E01v9 sdAb: | EVQLLESGGGEVQPGGSLRLSCAASGWAFGNYGMAWFRQA |
| | SEQ ID IN:37 of U.S. | PGKEREFVSRLAWNGGSTDYVESVKGRFTISRDNAKNTLY |
| | Publication No. | LQMSSLRAEDTAVYYCARQRSYSRYDIRTPQTYDYWGQGT |
| | 2017/0198050 A1 | LVTVKP (SEQ ID NO: 184) |
| 4-1BB | Hz4E01v10 sdAb: | EVQLLESGGGEVQPGGSLRLSCAASGWAFGNYGMAWFRQA |
| | SEQ ID IN:38 of U.S. | PGKEREFVSRLAWNGGSTDYVESVKGRFTISRDNPKNTLY |
| | Publication No. | LQMSSLRAEDTAVYYCARQRSYSRYDIRTPQTYDYWGQGT |
| | 2017/0198050 A1 | LVTVKP (SEQ ID NO: 185) |
| 4-1BB | Hz4E01v11 sdAb: | EVQLLESGGGEVQPGGSLRLSCAASGWAFGNYGMAWFRQA |
| | SEQ ID IN:39 of U.S. | PGKEREFIGRLAWNGGSTDYVESVKGRFTISRDNAKNTLY |
| | Publication No. | LQMSSLRAEDTAVYYCARQRSYSRYDIRTPQTYDYWGQGT |
| | 2017/0198050 A1 | LVTVKP (SEQ ID NO: 186) |
| 4-1BB | Hz4E01v12 sdAb: | EVQLLESGGGEVQPGGSLRLSCAASGWAFGNYGMAWFRQA |
| | SEQ ID IN:40 of U.S. | PGKEREFIGRLAWNGGSTDYVESVKGRFTISRDNPKNTLY |
| | Publication No. | LQMSSLRAEDTAVYYCARQRSYSRYDIRTPQTYDYWGQGT |
| | 2017/0198050 A1 | LVTVKP (SEQ ID NO: 183) |
| 4-1BB | Hz4E01v13 sdAb: | EVQLLESGGGEVQPGGSLRLSCAASGWAFGNYGMAWFRQA |
| | SEQ ID IN:41 of U.S. | PGKEREFIGRLAWQGGSTDYVESVKGRFTISRDNPKNTLY |
| | Publication No. | LQMSSLRAEDTAVYYCARQRSYSRYDIRTPQTYDYWGQGT |
| | 2017/0198050 A1 | LVTVKP (SEQ ID NO: 187) |
| 4-1BB | Hz4E01v14 sdAb: | EVQLLESGGGEVQPGGSLRLSCAASGWAFGNYGMAWFRQA |
| | SEQ ID IN:43 of U.S. | PGKEREFIGRLAWNAGSTDYVESVKGRFTISRDNPKNTLY |
| | Publication No. | LQMSSLRAEDTAVYYCARQRSYSRYDIRTPQTYDYWGQGT |
| | 2017/0198050 A1 | LVTVKP (SEQ ID NO: 188) |
| 4-1BB | Hz4E01v16 sdAb: | EVQLLESGGGEVQPGGSLRLSCAASGWAFGNYGMAWFRQA |
| | SEQ ID IN:45 of U.S. | PGKEREFVSRLAWQGGSTDYVESVKGRFTISRDNAKNTLY |
| | Publication No. | LQMSSLRAEDTAVYYCARQRSYSRYDIRTPQTYDYWGQGT |
| | 2017/0198050 A1 | LVTVKP (SEQ ID NO: 189) |
| 4-1BB | Hz4E01v17 sdAb: | EVQLLESGGGEVQPGGSLRLSCAASGWAFGNYGMAWFRQA |
| | SEQ ID IN:46 of U.S. | PGKEREFVSRLAWNAGSTDYVESVKGRFTISRDNAKNTLY |
| | Publication No. | LQMSSLRAEDTAVYYCARQRSYSRYDIRTPQTYDYWGQGT |
| | 2017/0198050 A1 | LVTVKP (SEQ ID NO: 190) |
| 4-1BB | Hz4E01v18 sdAb: | EVQLLESGGGEVQPGGSLRLSCAASGWAFGNYGMAWFRQA |
| | SEQ ID IN:47 of U.S. | PGKEREFVSRLAWGGGSTDYVESVKGRFTISRDNAKNTLY |
| | Publication No. | LQMSSLRAEDTAVYYCARQRSYSRYDIRTPQTYDYWGQGT |
| | 2017/0198050 A1 | LVTVKP (SEQ ID NO: 191) |
| 4-1BB | Hz4E01v21 sdAb: | EVQLLESGGGEVQPGGSLRLSCAASGWAFSNYGMAWFRQA |
| | SEQ ID IN:49 of U.S. | PGKEREFVSRLAWGGGSTDYVESVKGRFTISRDNAKNTLY |
| | Publication No. | LQMSSLRAEDTAVYYCARQRSYSRYDIRTPQTYDYWGQGT |
| | 2017/0198050 A1 | LVTVKP (SEQ ID NO: 192) |
| 4-1BB | Hz4E01v22 sdAb: | EVQLLESGGGEVQPGGSLRLSCAASGWAFGNYGMAWFRQA |
| | SEQ ID IN:51 of U.S. | PGKEREFVSRLAWSGGSTDYVESVKGRFTISRDNAKNTLY |
| | Publication No. | LQMSSLRAEDTAVYYCARQRSYSRYDIRTPQTYDYWGQGT |
| | 2017/0198050 A1 | LVTVKP (SEQ ID NO: 193) |
| 4-1BB | Hz4E01v23 sdAb: | EVQLLESGGGEVQPGGSLRLSCAASGWAFSNYGMAWFRQA |
| | SEQ ID IN:53 of U.S. | PGKEREFVSRLAWSGGSTDYVESVKGRFTISRDNAKNTLY |
| | Publication No. | LQMSSLRAEDTAVYYCARQRSYSRYDIRTPQTYDYWGQGT |
| | 2017/0198050 A1 | LVTVKP (SEQ ID NO: 194) |
| 4-1BB | Hz4E01v24 sdAb: | EVQLLESGGGEVQPGGSLRLSCAASGWAFGNYGMAWFRQA |
| | SEQ ID IN:54 of U.S. | PGKEREFVSRLAWGGGSTDYVESVKGRFTISRDNAKNTLY |
| | Publication No. | LQMSSLRAEDTAVYYCARQRSYSGYDIRTPQTYDYWGQGT |
| | 2017/0198050 A1 | LVTVKP (SEQ ID NO: 195) |
| 4-1BB | Hz4E01v25 sdAb: | EVQLLESGGGEVQPGGSLRLSCAASGWAFGNYGMAWFRQA |
| | SEQ ID IN:56 of U.S. | PGKEREFVSRLAWGGGSTDYVESVKGRFTISRDNAKNTLY |
| | Publication No. | LQMSSLRAEDTAVYYCARQRSYSRYGIRTPQTYDYWGQGT |
| | 2017/0198050 A1 | LVTVKP (SEQ ID NO: 196) |
| 4-1BB | Hz4E01v26 sdAb: | EVQLLESGGGEVQPGGSLRLSCAASGWAFGNYGMAWFRQA |
| | SEQ ID IN:58 of U.S. | PGKEREFVSRLAWGGGSTDYVESVKGRFTISRDNAKNTLY |
| | Publication No. | LQMSSLRAEDTAVYYCARQRSYSGYGIRTPQTYDYWGQGT |
| | 2017/0198050 A1 | LVTVKP (SEQ ID NO: 197) |
| 4-1BB | HzRH3-v1 sdAb: | EVQLLESGGGEVQPGGSLRLSCAASGFSFSINAMGWYRQA |
| | SEQ ID IN:59 of U.S. | PGKGLEWVAAIDSGRNTVYAESVKGRFTISRDNAKNTLYL |
| | Publication No. | QMSSLRAEDTAVYYCGLLKGNRVVSPSVAYWGQGTLVTVK |
| | 2017/0198050 A1 | P (SEQ ID NO: 198) |
| 4-1BB | HzRH3v5-1 sdAb: | EVQLLESGGGEVQPGGSLRLSCAASGFSFSINAMGWYRQA |
| | SEQ ID IN:60 of U.S. | PGKRREFVAAIESGRNTVYAESVKGRFTISRDNAKNTVYL |
| | Publication No. | QMSSLRAEDTAVYYCGLLKGNRVVSPSVAYWGQGTLVTVK |
| | 2017/0198050 A1 | P (SEQ ID NO: 199) |
| 4-1BB | HzRH3v5-2 sdAb: | EVQLLESGGGEVQPGGSLRLSCAASGFSFSINAMGWYRQA |
| | SEQ ID IN:62 of U.S. | PGKRREFVAAIYSGRNTVYAESVKGRFTISRDNAKNTVYL |
| | Publication No. | QMSSLRAEDTAVYYCGLLKGNRVVSPSVAYWGQGTLVTVK |
| | 2017/0198050 A1 | P (SEQ ID NO: 200) |
| 4-1BB | HzRH3v5-3 sdAb: | EVQLLESGGGEVQPGGSLRLSCAASGFTFSINAMGWYRQA |
| | SEQ ID IN:64 of U.S. | PGKRREFVAAIESGRNTVYAESVKGRFTISRDNAKNTVYL |
| | Publication No. | QMSSLRAEDTAVYYCGLLKGNRVVSPSVAYWGQGTLVTVK |
| | 2017/0198050 A1 | P (SEQ ID NO: 201) |
| 4-1BB | HzRH3v5-6 sdAb: | EVQLLESGGGEVQPGGSLRLSCAASGFSFSINAMSWYRQA |
| | SEQ ID IN:66 of U.S. | PGKRREFVAAIESGRNTVYAESVKGRFTISRDNAKNTVYL |
| | Publication No. | QMSSLRAEDTAVYYCGLLKGNRVVSPSVAYWGQGTLVTVK |
| | 2017/0198050 A1 | P (SEQ ID NO: 202) |
| 4-1BB | HzRH3v5-8 sdAb: | EVQLLESGGGEVQPGGSLRLSCAASGFTFSSNAMGWYRQA |
| | SEQ ID IN:68 of U.S. | PGKRREFVAAIESGRNTVYAESVKGRFTISRDNAKNTVYL |
| | Publication No. | QMSSLRAEDTAVYYCGLLKGNRVVSPSVAYWGQGTLVTVK |
| | 2017/0198050 A1 | P (SEQ ID NO: 203) |
| 4-1BB | HzRH3v5-10 sdAb: | EVQLLESGGGEVQPGGSLRLSCAASGFSFSINAMGWYRQA |
| | SEQ ID IN:70 of U.S. | PGKRREFVAAIESSRNTVYAESVKGRFTISRDNAKNTVYL |
| | Publication No. | QMSSLRAEDTAVYYCGLLKGNRVVSPSVAYWGQGTLVTVK |
| | 2017/0198050 A1 | P (SEQ ID NO: 204) |
| 4-1BB | HzRH3v5-12 sdAb: | EVQLLESGGGEVQPGGSLRLSCAASGFSFSINAMGWYRQA |
| | SEQ ID IN:72 of U.S. | PGKRREFVAAIESGSNTVYAESVKGRFTISRDNAKNTVYL |
| | Publication No. | QMSSLRAEDTAVYYCGLLKGNRVVSPSVAYWGQGTLVTVK |
| | 2017/0198050 A1 | P (SEQ ID NO: 205) |
| 4-1BB | HzRH3v5-14 sdAb: | EVQLLESGGGEVQPGGSLRLSCAASGFSFSINAMGWYRQA |
| | SEQ ID IN:74 of U.S. | PGKRREFVAAIESGRSTVYAESVKGRFTISRDNAKNTVYL |
| | Publication No. | QMSSLRAEDTAVYYCGLLKGNRVVSPSVAYWGQGTLVTVK |
| | 2017/0198050 A1 | P (SEQ ID NO: 206) |
| 4-1BB | HzRH3v5-15 sdAb: | EVQLLESGGGEVQPGGSLRLSCAASGFSFSINAMGWYRQA |
| | SEQ ID IN:76 of U.S. | PGKRREFVAAIESGRNTYYAESVKGRFTISRDNAKNTVYL |
| | Publication No. | QMSSLRAEDTAVYYCGLLKGNRVVSPSVAYWGQGTLVTVK |
| | 2017/0198050 A1 | P (SEQ ID NO: 207) |
| 4-1BB | HzRH3v5-16 sdAb: | EVQLLESGGGEVQPGGSLRLSCAASGFSFSINAMGWYRQA |
| | SEQ ID IN:78 of U.S. | PGKRREFVAAIYSGSSTVYAESVKGRFTISRDNAKNTVYL |
| | Publication No. | QMSSLRAEDTAVYYCGLLKGNRVVSPSVAYWGQGTLVTVK |
| | 2017/0198050 A1 | P (SEQ ID NO: 208) |
| 4-1BB | HzRH3v7 sdAb: | EVQLLESGGGEVQPGGSLRLSCAVSGFSFSINAMGWYRQA |
| | SEQ ID IN:80 of U.S. | PGKRREFVAAIESGRNTVYAESVKGRFTISRDNAKNTVYL |
| | Publication No. | QMSSLRAEDTAVYYCGLLKGNRVVSPSVAYWGQGTLVTVK |
| | 2017/0198050 A1 | P (SEQ ID NO: 209) |
| 4-1BB | HzRH3v8 sdAb: | EVQLLESGGGEVQPGGSLRLSCAASGFSFSINAMGWYRQA |
| | SEQ ID IN:81 of U.S. | PGKRREFVAAIESGRNTVYAVSVKGRFTISRDNAKNTVYL |
| | Publication No. | QMSSLRAEDTAVYYCGLLKGNRVVSPSVAYWGQGTLVTVK |
| | 2017/0198050 A1 | P (SEQ ID NO: 210) |
| 4-1BB | HzRH3v9 sdAb: | EVQLLESGGGEVQPGGSLRLSCAASGFSFSINAMGWYRQA |
| | SEQ ID IN:82 of U.S. | PGKGREFVAAIESGRNTVYAESVKGRFTISRDNAKNTVYL |
| | Publication No. | QMSSLRAEDTAVYYCGLLKGNRVVSPSVAYWGQGTLVTVK |
| | 2017/0198050 A1 | P (SEQ ID NO: 211) |
| 4-1BB | HzRH3v13 sdAb: | EVQLLESGGGEVQPGGSLRLSCAASGFSFSINAMGWYRQA |
| | SEQ ID IN:83 of U.S. | PGKRREFLAAIESGRNTVYAESVKGRFTISRDNAKNTVYL |
| | Publication No. | QMSSLRAEDTAVYYCGLLKGNRVVSPSVAYWGQGTLVTVK |
| | 2017/0198050 A1 | P (SEQ ID NO: 212) |
| CD30 | AS47863 sdAb: | QVQLEESGGGSVQAGETLRLSCTASGSTFGDSDMGWYRQA |
| | SEQ ID NO: 9 of U.S. | PGNACELVSIISSDGRTYYVDSVKGRFTISQDNAVSTVYL |
| | Publication No. | QMNSLKPEDTGVYYCAADLRQYCRDGRCCGYWGQGTQVTV |
| | 2022/0144960 A1 | SS (SEQ ID NO: 213) |
| CD30 | AS48433 sdAb: | QIQLVESGGGSVQAGETLRLSCTASGSTFGDSDMGWYRQA |
| | SEQ ID NO: 10 of U.S. | PGNACELVSIISSDGRTYYVDSVKGRFTISQDNAVSTVYL |
| | Publication No. | QMNSLNPEDTGVYYCAADLRLNCRDGRCCGYWGQGTQVTV |
| | 2022/0144960 A1 | SS (SEQ ID NO: 214) |
| CD30 | AS48463 sdAb: | QVHLMESGGGSVQAGETLRLSCTASGFTFANSDMGWYRQA |
| | SEQ ID NO: 11 of U.S. | PGNACELVSIISSHGGTTYYVDSVKGRFTISRHNAENTVY |
| | Publication No. | LRMTSLKPEDTALYYCVADPRSNCRGGYCCGYWGPGTQVT |
| | 2022/0144960 A1 | VSS (SEQ ID NO: 215) |
| CD30 | AS48481 sdAb: | EVQLVASGGGSVQAGETLRLSCTASGFTFADSAMGWYRKG |
| | SEQ ID NO: 12 of U.S. | PGNVCDLVAIIRTDGTTYYGDSAKGRFTISRDNAKSTLYL |
| | Publication No. | QMNSLKPEDTAVYFCAADRETSFIGGSWCVAKYWDQGTQV |
| | 2022/0144960 A1 | TVSS (SEQ ID NO: 216) |
| CD30 | AS48508 sdAb: | EVQLVESGGGSVQAGGSLRLSCTASRFTFDGPDMAWYRQA |
| | SEQ ID NO: 13 of U.S. | PGNACELVSIISADGRTYYTDSVKGRFTISRDNAKNTVFL |
| | Publication No. | YLNSLQPEDTAVYYCAPDPRRNCRGGYCCGNWGPGTQVTV |
| | 2022/0144960 A1 | SS (SEQ ID NO: 217) |
| CD30 | AS48542 sdAb: | QMQLVESGGGSVQAGETLRLSCTTSAFTFDGPDMAWYRQA |
| | SEQ ID NO: 14 of U.S. | PGNECVLVSIISADGRTYYADSVKGRFTISRDNAKNTVFL |
| | Publication No. | NLNSLQPEDTAVYYCALDPRKNCRGGYCCANWGPGTQVTV |
| | 2022/0144960 A1 | SS (SEQ ID NO: 218) |
| CD30 | AS53445 sdAb: | QVQLVESGGGSVQAGGSLRLSCTASGYIFCMGWFRQAPGK |
| | SEQ ID NO: 15 of U.S. | AREGIATIYTGGDSTYYDDSVKGRFTISRDNAKNTVYLQM |
| | Publication No. | NSLKPEDTAMYYCAAGGQECYLTNWVSYWGQGTQVTVSS |
| | 2022/0144960 A1 | (SEQ ID NO: 219) |
| CD30 | AS53574 sdAb: | QVKLVESGGGSVQAGGSLRLSCAASGYIYSSNCMGWFRQA |
| | SEQ ID NO: 16 of U.S. | PGKEREWVARIHTGSGSTYYADSVKGRFTISQDNAKNTVY |
| | Publication No. | LQMNSLRPEDTAMYDCAAGRVVLGAVVCTNEYWGQGTQVT |
| | 2022/0144960 A1 | VSS (SEQ ID NO: 220) |
| CD30 | AS53750 sdAb: | EVQLVESGGGLVQPGGSLRLSCTASGFTDDGPDMAWYRRA |
| | SEQ ID NO: 17 of U.S. | PGNECELVSIISADGRTYYTDSVKGRFTISRDNAKNTVFL |
| | Publication No. | YLNSLQPEDTAVYYCAPDPRRNCRGGDCCGNWGPGTQVTV |
| | 2022/0144960 A1 | SS (SEQ ID NO: 221) |
| CD30 | AS54233 sdAb: | QVQLVESGGGSVQAGETLRLSCTASGFTFDGPDMAWYRQA |
| | SEQ ID NO: 18 of U.S. | PGNECELVSIISADGRTYYTDSVKGRFTASQDNAKNTVSL |
| | Publication No. | YLKSLQPEDTAVYYCAADPRRNCRGNCCGNWGPGTQVTVS |
| | 2022/0144960 A1 | S (SEQ ID NO: 222) |
| CD30 | AS47863VH4 sdAb: | EVQLVESGGGLVQPGGSLRLSCAASGSTFGDSDMGWYRQA |
| | SEQ ID NO: 19 of U.S. | PGKGCELVSIISSDGRTYYVDSVKGRFTISQDNSKNTLYL |
| | Publication No. | QMNSLRAEDTAVYYCAADLRQYCRDGRCCGYWGQGTLVTV |
| | 2022/0144960 A1 | SS (SEQ ID NO: 223) |
| CD30 | AS 47863VH5 sdAb: | EVQLVESGGGLVQPGGSLRLSCAASGSTFGDSDMGWYRQA |
| | SEQ ID NO: 20 of U.S. | PGKGCELVSIISSDGRTYYVDSVKGRFTISQDNSKNTVYL |
| | Publication No. | QMNSLRAEDTAVYYCAADLRQYCRDGRCCGYWGQGTLVTV |
| | 2022/0144960 A1 | SS (SEQ ID NO: 224) |
| CD30 | AS47863VH11 sdAb: | EVQLVESGGGLVQPGGSLRLSCAASGSTFGDSDMGWYRQA |
| | SEQ ID NO: 21 of U.S. | PGKGCELVSIISSDGRTYYVDSVKGRFTISQDNAKNTLYL |
| | Publication No. | QMNSLRPEDTAVYYCAADLRQYCRDGRCCGYWGQGTLVTV |
| | 2022/0144960 A1 | SS (SEQ ID NO: 225) |
| CD30 | AS47863VH12 sdAb: | EVQLVESGGGLVQPGGSLRLSCAASGSTFGDSDMGWYRQA |
| | SEQ ID NO: 22 of U.S. | PGKGCELVSIISSDGRTYYVDSVKGRFTISQDNAKNTVYL |
| | Publication No. | QMNSLRPEDTAVYYCAADLRQYCRDGRCCGYWGQGTLVTV |
| | 2022/0144960 A1 | SS (SEQ ID NO: 226) |
| CD30 | AS48433VH4 sdAb: | EVQLVESGGGLVQPGGSLRLSCAASGSTFGDSDMGWYRQA |
| | SEQ ID NO: 23 of U.S. | PGKGCELVSIISSDGRTYYVDSVKGRFTISQDNSKNTLYL |
| | Publication No. | QMNSLRAEDTAVYYCAADLRLNCRDGRCCGYWGQGTLVTV |
| | 2022/0144960 A1 | SS (SEQ ID NO: 227) |
| CD30 | AS48433VH5 sdAb: | EVQLVESGGGLVQPGGSLRLSCAASGSTFGDSDMGWYRQA |
| | SEQ ID NO: 24 of U.S. | PGKGCELVSIISSDGRTYYVDSVKGRFTISQDNSKNTVYL |
| | Publication No. | QMNSLRAEDTAVYYCAADLRLNCRDGRCCGYWGQGTLVTV |
| | 2022/0144960 A1 | SS (SEQ ID NO: 228) |
| CD30 | AS48433VH11 sdAb: | EVQLVESGGGLVQPGGSLRLSCAASGSTFGDSDMGWYRQA |
| | SEQ ID NO: 25 of U.S. | PGKGCELVSIISSDGRTYYVDSVKGRFTISQDNAKNTLYL |
| | Publication No. | QMNSLRPEDTAVYYCAADLRLNCRDGRCCGYWGQGTLVTV |
| | 2022/0144960 A1 | SS (SEQ ID NO: 229) |
| CD30 | AS48433VH12 sdAb: | EVQLVESGGGLVQPGGSLRLSCAASGSTFGDSDMGWYRQA |
| | SEQ ID NO: 26 of U.S. | PGKGCELVSIISSDGRTYYVDSVKGRFTISQDNAKNTVYL |
| | Publication No. | QMNSLRPEDTAVYYCAADLRLNCRDGRCCGYWGQGTLVTV |
| | 2022/0144960 A1 | SS (SEQ ID NO: 230) |
| CD30 | AS48463VH4 sdAb: | EVQLLESGGGLVQPGGSLRLSCAASGFTFANSDMGWYRQA |
| | SEQ ID NO: 27 of U.S. | PGKGCELVSIISSHGGTTYYVDSVKGRFTISRDNSKNTLY |
| | Publication No. | LQMNSLRAEDTAVYYCVADPRSNCRGGYCCGYWGQGTLVT |
| | 2022/0144960 A1 | VSS (SEQ ID NO: 231) |
| CD30 | AS48463VH11 sdAb: | EVQLLESGGGLVQPGGSLRLSCAASGFTFANSDMGWYRQA |
| | SEQ ID NO: 28 of U.S. | PGKGCELVSIISSHGGTTYYVDSVKGRFTISRDNAKNTLY |
| | Publication No. | LQMNSLRPEDTAVYYCVADPRSNCRGGYCCGYWGQGTLVT |
| | 2022/0144960 A1 | VSS (SEQ ID NO: 232) |
| CD30 | AS48481VH5 sdAb: | QVQLVESGGGVVQPGRSLRLSCAASGFTFADSAMGWYRQA |
| | SEQ ID NO: 29 of U.S. | PGKGCELVAIIRTDGTTYYGDSAKGRFTISRDNSKNTLYL |
| | Publication No. | QMNSLRAEDTAVYFCAADRETSFIGGSWCVAKYWDQGTLV |
| | 2022/0144960 A1 | TVSS (SEQ ID NO: 233) |
| CD30 | AS48481VH6 sdAb: | QVQLVESGGGVVQPGRSLRLSCAASGFTFADSAMGWYRQA |
| | SEQ ID NO: 30 of U.S. | PGKVCELVAIIRTDGTTYYGDSAKGRFTISRDNSKNTLYL |
| | Publication No. | QMNSLRAEDTAVYFCAADRETSFIGGSWCVAKYWDQGTLV |
| | 2022/0144960 A1 | TVSS (SEQ ID NO: 234) |
| CD30 | AS48481VH13 sdAb: | QVQLVESGGGVVQPGRSLRLSCAASGFTFADSAMGWYRQA |
| | SEQ ID NO: 31 of U.S. | PGKGCELVAIIRTDGTTYYGDSAKGRFTISRDNAKNTLYL |
| | Publication No. | QMNSLRPEDTAVYFCAADRETSFIGGSWCVAKYWDQGTLV |
| | 2022/0144960 A1 | TVSS (SEQ ID NO: 235) |
| CD30 | AS48481VH14 sdAb: | QVQLVESGGGVVQPGRSLRLSCAASGFTFADSAMGWYRQA |
| | SEQ ID NO: 32 of U.S. | PGKVCELVAIIRTDGTTYYGDSAKGRFTISRDNAKNTLYL |
| | Publication No. | QMNSLRPEDTAVYFCAADRETSFIGGSWCVAKYWDQGTLV |
| | 2022/0144960 A1 | TVSS (SEQ ID NO: 236) |
| CD30 | AS48508VH4 sdAb: | EVQLVESGGGLVQPGGSLRLSCAASRFTFDGPDMAWYRQA |
| | SEQ ID NO: 33 of U.S. | PGKGCELVSIISADGRTYYTDSVKGRFTISRDNSKNTLYL |
| | Publication No. | QMNSLRAEDTAVYYCAPDPRRNCRGGYCCGNWGQGTTVTV |
| | 2022/0144960 A1 | SS (SEQ ID NO: 237) |
| CD30 | AS48508VH5 sdAb: | EVQLVESGGGLVQPGGSLRLSCAASRFTFDGPDMAWYRQA |
| | SEQ ID NO: 34 of U.S. | PGKGCELVSIISADGRTYYTDSVKGRFTISRDNSKNTVYL |
| | Publication No. | QMNSLRAEDTAVYYCAPDPRRNCRGGYCCGNWGQGTTVTV |
| | 2022/0144960 A1 | SS (SEQ ID NO: 238) |
| CD30 | AS48508VH11 sdAb: | EVQLVESGGGLVQPGGSLRLSCAASRFTFDGPDMAWYRQA |
| | SEQ ID NO: 35 of U.S. | PGKGCELVSIISADGRTYYTDSVKGRFTISRDNAKNTLYL |
| | Publication No. | QMNSLRPEDTAVYYCAPDPRRNCRGGYCCGNWGQGTTVTV |
| | 2022/0144960 A1 | SS (SEQ ID NO: 239) |
| CD30 | AS48508VH12 sdAb: | EVQLVESGGGLVQPGGSLRLSCAASRFTFDGPDMAWYRQA |
| | SEQ ID NO: 36 of U.S. | PGKGCELVSIISADGRTYYTDSVKGRFTISRDNAKNTVYL |
| | Publication No. | QMNSLRPEDTAVYYCAPDPRRNCRGGYCCGNWGQGTTVTV |
| | 2022/0144960 A1 | SS (SEQ ID NO: 240) |
| CD30 | AS48542VH5 sdAb: | EVQLVESGGGLVQPGGSLRLSCATSAFTFDGPDMAWYRQA |
| | SEQ ID NO: 37 of U.S. | PGKGCELVSIISADGRTYYADSVKGRFTISRDNSKNTVYL |
| | Publication No. | QMNSLRAEDTAVYYCALDPRKNCRGGYCCANWGQGTLVTV |
| | 2022/0144960 A1 | SS (SEQ ID NO: 241) |
| CD30 | AS48542VH12 sdAb: | EVQLVESGGGLVQPGGSLRLSCATSAFTFDGPDMAWYRQA |
| | SEQ ID NO: 38 of U.S. | PGKGCELVSIISADGRTYYADSVKGRFTISRDNAKNTVYL |
| | Publication No. | QMNSLRPEDTAVYYCALDPRKNCRGGYCCANWGQGTLVTV |
| | 2022/0144960 A1 | SS (SEQ ID NO: 242) |
| CD30 | AS53445VH4 sdAb: | QVQLVESGGGVVQPGGSLRLSCAASGYIFCMGWFRQAPGK |
| | SEQ ID NO: 39 of U.S. | GLEGIATIYTGGDSTYYDDSVKGRFTISRDNSKNTLYLQM |
| | Publication No. | NSLRAEDTAVYYCAAGGQECYLTNWVSYWGQGTLVTVSS |
| | 2022/0144960 A1 | (SEQ ID NO: 243) |
| CD30 | AS53445VH11 sdAb: | QVQLVESGGGVVQPGGSLRLSCAASGYIFCMGWFRQAPGK |
| | SEQ ID NO: 40 of U.S. | GREGIATIYTGGDSTYYDDSVKGRFTISRDNAKNTLYLQM |
| | Publication No. | NSLRPEDTAVYYCAAGGQECYLTNWVSYWGQGTLVTVSS |
| | 2022/0144960 A1 | (SEQ ID NO: 244) |
| CD30 | AS53574VH4 sdAb: | EVQLVESGGGLVQPGGSLRLSCAASGYIYSSNCMGWFRQA |
| | SEQ ID NO: 41 of U.S. | PGKGLEWVSRIHTGSGSTYYADSVKGRFTISRDNSKNTLY |
| | Publication No. | LQMNSLRAEDTAVYYCAAGRVVLGAVVCTNEYWGQGTLVT |
| | 2022/0144960 A1 | VSS (SEQ ID NO: 245) |
| CD30 | AS53574VH5 sdAb: | EVQLVESGGGLVQPGGSLRLSCAASGYIYSSNCMGWFRQA |
| | SEQ ID NO: 42 of U.S. | PGKGLEWVSRIHTGSGSTYYADSVKGRFTISQDNSKNTLY |
| | Publication No. | LQMNSLRAEDTAVYYCAAGRVVLGAVVCTNEYWGQGTLVT |
| | 2022/0144960 A1 | VSS (SEQ ID NO: 246) |
| CD30 | AS53574VH6 sdAb: | EVQLVESGGGLVQPGGSLRLSCAASGYIYSSNCMGWFRQA |
| | SEQ ID NO: 43 of U.S. | PGKGLEWVARIHTGSGSTYYADSVKGRFTISQDNSKNTLY |
| | Publication No. | LQMNSLRAEDTAVYYCAAGRVVLGAVVCTNEYWGQGTLVT |
| | 2022/0144960 A1 | VSS (SEQ ID NO: 247) |
| CD30 | AS53574VH11 sdAb: | EVQLVESGGGLVQPGGSLRLSCAASGYIYSSNCMGWFRQA |
| | SEQ ID NO: 44 of U.S. | PGKGREWVSRIHTGSGSTYYADSVKGRFTISRDNAKNTLY |
| | Publication No. | LQMNSLRPEDTAVYYCAAGRVVLGAVVCTNEYWGQGTLVT |
| | 2022/0144960 A1 | VSS (SEQ ID NO: 248) |
| CD30 | AS53574VH12 sdAb: | EVQLVESGGGLVQPGGSLRLSCAASGYIYSSNCMGWFRQA |
| | SEQ ID NO: 45 of U.S. | PGKGREWVSRIHTGSGSTYYADSVKGRFTISQDNAKNTLY |
| | Publication No. | LQMNSLRPEDTAVYYCAAGRVVLGAVVCTNEYWGQGTLVT |
| | 2022/0144960 A1 | VSS (SEQ ID NO: 249) |
| CD30 | AS53574VH13 sdAb: | EVQLVESGGGLVQPGGSLRLSCAASGYIYSSNCMGWFRQA |
| | SEQ ID NO: 46 of U.S. | PGKGREWVARIHTGSGSTYYADSVKGRFTISQDNAKNTLY |
| | Publication No. | LQMNSLRPEDTAVYYCAAGRVVLGAVVCTNEYWGQGTLVT |
| | 2022/0144960 A1 | VSS (SEQ ID NO: 250) |
| CD30 | AS53750VH4 sdAb: | EVQLVESGGGLVQPGGSLRLSCAASGFTDDGPDMAWYRQA |
| | SEQ ID NO: 47 of U.S. | PGKGCELVSIISADGRTYYTDSVKGRFTISRDNSKNTLYL |
| | Publication No. | QMNSLRAEDTAVYYCAPDPRRNCRGGDCCGNWGQGTTVTV |
| | 2022/0144960 A1 | SS (SEQ ID NO: 251) |
| CD30 | AS53750VH5 sdAb: | EVQLVESGGGLVQPGGSLRLSCAASGFTDDGPDMAWYRQA |
| | SEQ ID NO: 48 of U.S. | PGKGCELVSIISADGRTYYTDSVKGRFTISRDNSKNTVYL |
| | Publication No. | QMNSLRAEDTAVYYCAPDPRRNCRGGDCCGNWGQGTTVTV |
| | 2022/0144960 A1 | SS (SEQ ID NO: 252) |
| CD30 | AS53750VH11 sdAb: | EVQLVESGGGLVQPGGSLRLSCAASGFTDDGPDMAWYRQA |
| | SEQ ID NO: 49 of U.S. | PGKGCELVSIISADGRTYYTDSVKGRFTISRDNAKNTLYL |
| | Publication No. | QMNSLRPEDTAVYYCAPDPRRNCRGGDCCGNWGQGTTVTV |
| | 2022/0144960 A1 | SS (SEQ ID NO: 253) |
| CD30 | AS53750VH12 sdAb: | EVQLVESGGGLVQPGGSLRLSCAASGFTDDGPDMAWYRQA |
| | SEQ ID NO: 50 of U.S. | PGKGCELVSIISADGRTYYTDSVKGRFTISRDNAKNTVYL |
| | Publication No. | QMNSLRPEDTAVYYCAPDPRRNCRGGDCCGNWGQGTTVTV |
| | 2022/0144960 A1 | SS (SEQ ID NO: 254) |
| CD30 | AS54233VH4 sdAb: | EVQLVESGGGLVQPGGSLRLSCAASGFTFDGPDMAWYRQA |
| | SEQ ID NO: 51 of U.S. | PGKGCELVSIISADGRTYYTDSVKGRFTASQDNSKNTLYL |
| | Publication No. | QMNSLRAEDTAVYYCAADPRRNCRGNCCGNWGQGTLVTVS |
| | 2022/0144960 A1 | S (SEQ ID NO: 255) |
| CD30 | AS54233VH5 sdAb: | EVQLVESGGGLVQPGGSLRLSCAASGFTFDGPDMAWYRQA |
| | SEQ ID NO: 52 of U.S. | PGKGCELVSIISADGRTYYTDSVKGRFTASQDNSKNTVYL |
| | Publication No. | QMNSLRAEDTAVYYCAADPRRNCRGNCCGNWGQGTLVTVS |
| | 2022/0144960 A1 | S (SEQ ID NO: 256) |
| CD30 | AS54233VH11 sdAb: | EVQLVESGGGLVQPGGSLRLSCAASGFTFDGPDMAWYRQA |
| | SEQ ID NO: 53 of U.S. | PGKGCELVSIISADGRTYYTDSVKGRFTASQDNAKNTLYL |
| | Publication No. | QMNSLRPEDTAVYYCAADPRRNCRGNCCGNWGQGTLVTVS |
| | 2022/0144960 A1 | S (SEQ ID NO: 257) |
| CD30 | AS54233VH12 sdAb: | EVQLVESGGGLVQPGGSLRLSCAASGFTFDGPDMAWYRQA |
| | SEQ ID NO: 54 of U.S. | PGKGCELVSIISADGRTYYTDSVKGRFTASQDNAKNTVYL |
| | Publication No. | QMNSLRPEDTAVYYCAADPRRNCRGNCCGNWGQGTLVTVS |
| | 2022/0144960 A1 | S (SEQ ID NO: 258) |
| HVEM | VHH Ab#1: | QVQLVESGGGLVQPGGSLRLSCAASGFTFSTHAMSWYRQA |
| | SEQ ID NO: 29 of U.S. | PGKERELVAHISSTGGSTNYADSVKGRFTISRDNAKNTVY |
| | Publication No. | LQMNSLKPEDTAVYRCNARDWYEYWGRGTQVTVSS (SEQ |
| | 2022/0105122 A1 | ID NO: 259) |
| HVEM | VHH Ab#2: | QVQLVESGGGLVQPGGSLRLSCVASGRLFDTYTMAWYRLP |
| | SEQ ID NO: 30 of U.S. | PGKQRELVADISRTGFTNYADSVKGRFTISRDNAKNTVYL |
| | Publication No. | QMNSLKPDDTAHYRCKVREPATMYEYWGQGTQVTVSS |
| | 2022/0105122 A1 | (SEQ ID NO: 260) |
| HVEM | VHH Ab#3: | QVQLVESGGGSVQPGGSLRLSCAASGSIGSIHRWDWYRLC |
| | SEQ ID NO: 31 of U.S. | PGKQREWVATLNSEGGPTYADSVEGRFTISRDNAKNMVYL |
| | Publication No. | QMNSLKPEDTAVYRCSARTVPFEYWGRGTQVTVSS (SEQ |
| | 2022/0105122 A1 | ID NO: 261) |
| HVEM | VHH Ab#4: | QVQLVESGGGLVQPGGSLRLSCAAAESMLSPYSMGWYRLP |
| | SEQ ID NO: 32 of U.S. | PGKQRELVASLGSGGRTTYADSVKGRFTISRDNAKNTAYL |
| | Publication No. | QMNSLKPEDTAVYRCNIRELLGRRYEYWSQGTQVTVSS |
| | 2022/0105122 A1 | (SEQ ID NO: 262) |
| HVEM | VHH Ab#5: | QVQLVESGGGLVQPGGSLRLSCAASGFTFSSSHMTWVRQA |
| | SEQ ID NO: 33 of U.S. | PGKGLEWVSLINSSGRTAYVDSVAGRFTISRDNAKNTLYL |
| | Publication No. | QMTNLKPEDTAVYYCSKGGWDGLPSSSIRGQGTQVTVSS |
| | 2022/0105122 A1 | (SEQ ID NO: 263) |
| HVEM | VHH Ab#6: | QVQLVESGGGLVPIGGSLRLSCAVSGLAFDRYTFNWYRQA |
| | SEQ ID NO: 34 of U.S. | PGKGREWVASIDSTSAVIDYEDTVKGRFTISRDNTKNTVY |
| | Publication No. | LQMNSLKPEDTAVYFCGRGGYYGQGTQVTVST (SEQ ID |
| | 2022/0105122 A1 | NO: 264) |
| HVEM | VHH Ab#7: | QVQLVESGGGLVQPGGSLKLSCVASGFTDKPTYWSWYRQP |
| | SEQ ID NO: 35 of U.S. | PGKDRELVARMHTGGLGTAYPDSVAGRFTISTVNDKNTVY |
| | Publication No. | LQMNSLKPEDTAVYYCNAEISGGPDYWGQGTQVTVSS |
| | 2022/0105122 A1 | (SEQ ID NO: 265) |
| IGF1R | IGF1 R-5: | QVKLEESGGGLVQAGGSLRLSCAASGRTIDNYAMAWSRQA |
| | SEQ ID NO: 5 of PCT | PGKDREFVATIDWGDGGARYANSVKGRFTISRDNAKGTMY |
| | Publication No. WO | LQMNNLEPEDTAVYSCAMARQSRVNLDVARYDYWGQGTQV |
| | 2015/131258 A1 | TVSS (SEQ ID NO: 266) |
| IGF1R | IGF1 R-5_H1: | EVQLVESGGGLVQPGGSLRLSCAASGRTIDNYAMAWVRQA |
| | SEQ ID NO: 6 of PCT | PGKGLEWVSTIDWGDGGTRYANSVKGRFTISRDNSKNTLY |
| | Publication No. WO | LQMNSLRAEDTAVYYCAMARQSRVNLDVARYDYWGQGTLV |
| | 2015/131258 A1 | TVSS (SEQ ID NO: 267) |
| IGF1R | IGF1 R-5_H2: | QVQLVESGGGLVQPGGSLRLSCAASGRTIDNYAMAWVRQA |
| | SEQ ID NO: 7 of PCT | PGKGLEWVATIDWGDGGTRYANSVKGRFTISRDNSKNTMY |
| | Publication No. WO | LQMNSLRAEDTAVYYCAMARQSRVNLDVARYDYWGQGTLV |
| | 2015/131258 A1 | TVSS (SEQ ID NO: 268) |
| IGF1R | IGF1 R-5_H3: | QVQLVESGGGLVQPGGSLRLSCAASGRTIDNYAMAWSRQA |
| | SEQ ID NO: 8 of PCT | PGKGLEFVATIDWGDGGTRYANSVKGRFTISRDNSKNTMY |
| | Publication No. WO | LQMNSLRAEDTAVYYCAMARQSRVNLDVARYDYWGQGTLV |
| | 2015/131258 A1 | TVSS (SEQ ID NO: 269) |
| IGF1R | IGF1 R-5JH5: | QVQLVESGGGLVQPGGSLRLSCAASGRTIDNYAMAWSRQA |
| | SEQ ID NO: 9 of PCT | PGKDREFVATIDWGDGGTRYANSVKGRFTISRDNSKGTMY |
| | Publication No. WO | LQMNSLRAEDTAVYSCAMARQSRVNLDVARYDYWGQGTLV |
| | 2015/131258 A1 | TVSS (SEQ ID NO: 270) |
| IGF1R | IGF1 R-5JH6: | EVQLVESGGGLVQPGGSLRLSCAASGRTIDNYAMAWSRQA |
| | SEQ ID NO: 10 of PCT | PGKDREFVSTIDWGDGGTRYANSVKGRFTISRDNSKNTLY |
| | Publication No. WO | LQMNSLRAEDTAVYYCAMARQSRVNLDVARYDYWGQGTLV |
| | 2015/131258 A1 | TVSS (SEQ ID NO: 271) |
| IGF1R | PMP4B11 nanobody: | EVQLVESGGGLVQPGGSLRLSCAASGSIFTFNAMGWYRQA |
| | SEQ ID NO: 106 of | PGKQRELVAVIISGGSTHYVDSVKGRFTISRDNAKKMVYL |
| | U.S. Publication No. | QMNSLKPEDTAVYYCNVKKFGDYWGQGTQVTVSS (SEQ |
| | 2009/0252681 A1 | ID NO: 272) |
| IGF1R | PMP3G7 nanobody: | DVQLVESGGGLVQAGGSLRLSCAASESISTINVMAWYRQA |
| | SEQ ID NO: 107 of | PGKQRELVAEITRSGRTNYVDSVKGRFTISRDNAKNTMYL |
| | U.S. Publication No. | QMNSLNLEDTAVYYCRTIDGSWREYWGQGTQVTVSS |
| | 2009/0252681 A1 | (SEQ ID NO: 273) |
| IGF1R | PMP2C7 nanobody: | QVKLEESGGGLVQPGGSLRLSCVASGRTFSNYAMGWFRQA |
| | SEQ ID NO: 108 of | PGQEREFVAAINWNSRSTYYADSVKGRFTISRLNARNTVY |
| | U.S. Publication No. | LQMNRLKPEDTAVYDCAASHDSDYGGTNANLYDYWGQGTQ |
| | 2009/0252681 A1 | VTVSS (SEQ ID NO: 274) |
| IGF1R | PMP1C7 nanobody: | QVKLEESGGGLVQAGGSLRLSCVASGRTFSRTAMAWFRQA |
| | SEQ ID NO: 109 of | PGKEREFVATITWNSGTTRYADSVKGRFFISKDSAKNTIY |
| | U.S. Publication No. | LEMNSLEPEDTAVYYCAATAAAVITPTRGYYNYWGQGTQV |
| | 2009/0252681 A1 | TVSS (SEQ ID NO: 275) |
| CD27 | VHH | |
| | Abnova Cat no. | |
| | RAB06857 | |
| EpoR | EpoR VHH-8His-Cys- | |
| | tag Recombinant | |
| | Alpaca Monoclonal | |
| | Antibody Invitrogen | |
| | Cat No. MA5-54567 | |

6.2.4. sdAb Targeting Moiety Formats

In certain aspects, a targeting moiety can be any type of sdAb or fragment thereof that retains specific binding to an antigenic determinant. Antibody fragments include, but are not limited to VHH domains and sdVH domains.

In some embodiments, the targeting moiety of an engineered antigen-binding molecule share the same format (e.g., VHH, sdVH). In another embodiment, the targeting moieties do not share the same format.

A single-domain antibody (sdAb) describes a single antigen-binding domain capable of binding to a cognate antigen. sdAbs are often derived from naturally-occurring heavy-chain only antibodies (e.g., VHHs); however, they also include single VH or VL domains capable of binding to their cognate antigen in the absence of an associated light chain or heavy chain. Single VH or VL domains may have amino acid changes relative to native VH or VL sequences that stabilize the domains and/or reduce or eliminate aggregation.

Heavy-chain only antibodies (e.g., VHHs or sdVHs) lack light chains and thus rely exclusively on a heavy chain variable domain for antigen binding. Heavy-chain only antibodies are produced naturally in the Camelidae family (e.g., camels, dromedaries, llamas, vicunas, guanaco, and alpacas) as well as in cartilaginous fish (e.g., sharks). In addition to natural sources, transgenic mammals (e.g., mice) have been engineered to express heavy-chain only antibodies. Such transgenic mammals include, for example, transgenic animals described in U.S. Patent Publications 2015/0289489 A1, 2023/0270086 A1, and 2023/0062964 A1, and 2020/0267951 A1, each of which is incorporated herein by reference.

In some embodiments, an sdAb is generated by immunizing an animal that produces heavy-chain only antibodies, including a natural producer (e.g., camelids, sharks) or an engineered non-human mammal (e.g., a transgenic mouse), to obtain heavy-chain only antibodies. Such antibodies may be screened to identify those having desirable properties (e.g., target affinity). Once produced and identified, the variable region of the antibody heavy chain is cloned to construct a single domain antibody consisting of only one heavy chain variable region.

sdAbs can also be obtained by immunizing animals that generate traditional antibodies (e.g., rabbits) followed by screening for VHs having high binding affinity in the absence of their cognate light chain (see e.g., Shinozaki et al., 2017, Scientific Reports, 7(1):5794).

sdAbs can be humanized by replacing natural (e.g., camelid) framework sequences with human sequences (see, e.g., Vincke, 2009, The Journal of Biological Chemistry, 285(5):3273-3284; Murakami et al., 2022, Antibodies, 11(1):10; and U.S. Patent Publication No. 2016/0237142 A1, incorporated herein by reference). A sdVH may be fully human or may be a humanized sdVH derived from a non-human animal such as, for example, mouse or rabbit.

Fully human sdAbs (e.g., sdVHs) can also be obtained using human VH single domains (see, e.g., Rouet et al., 2015, The Journal of Biological Chemistry, 290(19):11905-11917).

Additional methods for producing heavy-chain only antibodies and/or sdAbs are recognized in the art and include, for example, those described in Muyldermans, 2021, The FEBS journal, 288(7):2084-2102.

In some cases, an sdAb is engineered to enhance certain properties. For example, in some embodiments, a disulfide bond is introduced within a VHH to increase stability (see e.g., Hagihara et al., 2007, The Journal of Biological Chemistry, 282(50):36489-36495).

### 6.2.5. Exemplary Engineered Antigen-binding Constructs

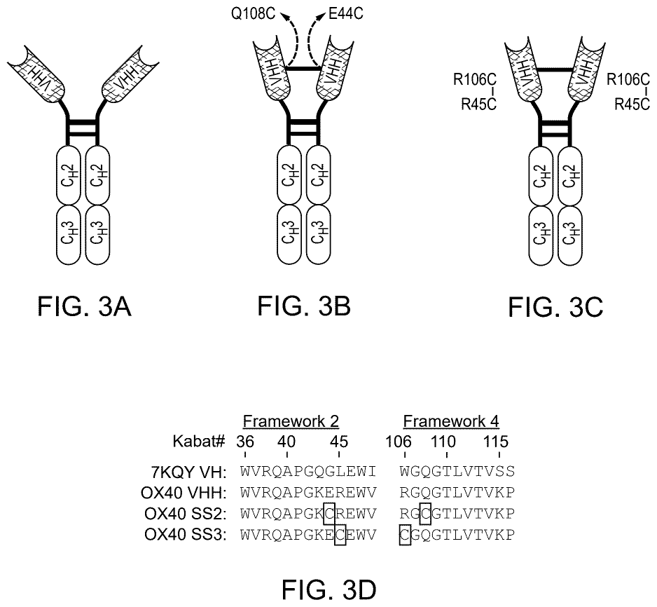

Components of engineered antigen-binding molecules described above can be combined in a variety of ways. Exemplary engineered antigen-binding molecules are illustrated in FIG. 1 and FIGS. 3A to 3C. The illustrated engineered antigen-binding molecules may include any of the hinge, CH2, or CH3 domains or targeting moieties disclosed above, including wild-type or variant versions thereof.

An exemplary engineered antigen-binding molecule is illustrated in FIG. 1A, which depicts a molecule comprising a first polypeptide chain and a second polypeptide chain, each comprising from N-terminus to C-terminus: an sdAb variable domain and an Fc domain (comprising from N-terminus to C-terminus, a hinge domain, a CH2 domain, and a CH3 domain). The hinge domains may comprise inter-chain disulfide bonds. The sdAb variable domains form an inter-chain association, which may be a covalent or non-covalent association, as described above.

Another exemplary engineered antigen-binding molecule is illustrated in FIG. 3B, which depicts a molecule comprising a first polypeptide chain and a second polypeptide chain, each comprising from N-terminus to C-terminus: a VHH domain and an Fc domain (comprising from N-terminus to C-terminus, a hinge domain, a CH2 domain, and a CH3 domain). The hinge domains may comprise inter-chain disulfide bonds. Engineered cysteine residues at position Q108 of one VHH domain is linked via a disulfide bond to an engineered cysteine residue at position E44 of the other VHH domain. In some embodiments, engineered antigen-binding molecules comprise sdVH domains or other sdAb variable domains in place of the VHH domains illustrated in FIG. 3B. In some embodiments, engineered antigen-binding molecules comprise other dimerization moieties in place of the Fc domains illustrated in FIG. 3B.

Another exemplary engineered antigen-binding molecule is illustrated in FIG. 3C, which depicts a molecule comprising a first polypeptide chain and a second polypeptide chain, each comprising from N-terminus to C-terminus: a VHH domain and an Fc domain (comprising from N-terminus to C-terminus, a hinge domain, a CH2 domain, and a CH3 domain). The hinge domains may comprise inter-chain disulfide bonds. Each VHH has an intra-chain disulfide bond (not shown) between an engineered cysteine at position R45 and an engineered cysteine at position R106. The VHH domains form a non-covalent association, depicted as a line between the VHH domains. In some embodiments, engineered antigen-binding molecules comprise sdVH domains or other sdAb variable domains in place of the VHH domains illustrated in FIG. 3C. In some embodiments, engineered antigen-binding molecules comprise other dimerization moieties in place of the Fc domains illustrated in FIG. 3C.

In some embodiments, engineered antigen-binding molecule targets OX40 the first VHH domain and the second VHH domain are anti-OX40 VHH domains. In some embodiments, the first VHH domain comprises engineered cysteine residues at positions 44 and 108 and the second VHH domain comprises engineered cysteine residues at positions 44 and 108, numbering according to Kabat. In some embodiments, the first VHH domain and the second VHH domain comprise engineered cysteine residues at positions 44 and 108, numbering according to Kabat, and each comprises or consists of an amino acid sequence having at least 80%, 85%, 90%, 95%, 99% sequence identity to SEQ ID NO:36 or having 100% sequence identity to SEQ ID NO:36. In some embodiments, the first VHH domain comprises engineered cysteine residues at positions 45 and 106 and the second VHH domain comprises engineered cysteine residues at positions 45 and 106, numbering according to Kabat. In some embodiments, the engineered antigen-binding molecule comprises a first polypeptide chain comprising a first anti-OX40 VHH and an Fc domain and a second polypeptide chain comprising a second anti-OX40 VHH and a second Fc domain, wherein the first polypeptide chain and the second polypeptide comprise engineered cysteine residues at positions 44 and 108 of the first anti-OX40 VHH and second anti-OX40 VHH, respectively, numbering according to Kabat, and each comprises or consists of an amino acid sequence having at least 80%, 85%, 90%, 95%, 99% sequence identity to SEQ ID NO: 35 or having 100% sequence identity to SEQ ID NO:35. In some embodiments, the first VHH domain and the second VHH domain comprise engineered cysteine residues at positions 45 and 106, numbering according to Kabat, and each comprises or consists of an amino acid sequence having at least 80%, 85%, 90%, 95%, 99% sequence identity to SEQ ID NO: 36 or having 100% sequence identity to SEQ ID NO:38. In some embodiments, the engineered antigen-binding molecule comprises a first polypeptide chain comprising a first anti-OX40 VHH and an Fc domain and a second polypeptide chain comprising a second anti-OX40 VHH and a second Fc domain, wherein the first polypeptide chain and the second polypeptide comprise engineered cysteine residues at positions 45 and 106 of the first anti-OX40 VHH and second anti-OX40 VHH, respectively, numbering according to Kabat, and each comprises or consists of an amino acid sequence having at least 80%, 85%, 90%, 95%, or 99% sequence identity to SEQ ID NO:37 or having 100% sequence identity to SEQ ID NO: 37.

### 6.3. Nucleic Acids and Host Cells

In another aspect, the disclosure provides nucleic acids encoding the engineered antigen-binding molecules of the disclosure and/or their individual components. In some embodiments, the components of engineered antigen-binding molecules are encoded by a single nucleic acid. In other embodiments, components of the engineered antigen-binding molecules are encoded by separate nucleic acids.

For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements, and separated by internal ribosome entry site (IRES) sequences allowing for translation into separate polypeptides.

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

### 6.3.1. Vectors

The disclosure provides vectors comprising nucleotide sequences encoding engineered antigen-binding molecules or components thereof described herein, for example one or two of the polypeptide chains of an engineered antigen-binding molecule. The vectors may include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection or other conventional techniques. Methods and conditions for culturing the resulting transfected cells and for recovering the expressed polypeptides are known to those skilled in the art, and may be varied or optimized depending upon the specific expression vector and mammalian host cell employed, based upon the present description.

### 6.3.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

6.3.3. Methods of Making Antigen-Binding Molecules with sdAb Dimers

The disclosure also provides methods of making antigen-binding molecules that include at least two sdAb arms that form a dimer pair. In some embodiments, the two sdAb arms of the antigen-binding molecule co-elute as a dimer in a size-exclusion chromatography (SEC) column after sdAb variable domains are proteolytically cleaved from an antigen-binding molecule construct. The sdAb dimers formed according to methods of the present disclosure can have any of the arrangements and sets of amino acid substitutions described in Section 6.2.1.

In some embodiments, a method of making an sdAb dimer comprises co-expressing two separate polypeptide chains in a host cell, each polypeptide chain having an sdAb variable domain with cysteine substitutions as described in Section 6.2.1 (e.g., an SS2 or SS3 set of substitutions). Each polypeptide chain may also have a dimerization moiety (e.g., an Fc domain). In some embodiments, co-expressing the polypeptide chains having cysteine-substituted sdAbs in a host cell results in formation of an antigen-binding molecule composed of the two polypeptide chains. The substituted cysteines in the sdAbs may engage in disulfide bond formation, which may be inter-chain or intra-chain disulfide bonds, and the sdAbs may associate with one another, either covalently (e.g., with an inter-chain disulfide bond) or non-covalently (e.g., by FR4 domain swapping).

An sdAb dimer may also form between two sdAbs that are on a single polypeptide chain. For example, two sdAbs having cysteine substitutions as described herein may be joined by a polypeptide linker or other intervening sequences. In such embodiments, a method of making an sdAb dimer may comprise expressing the single polypeptide chain in a host cell.

Methods of making an antigen-binding molecule that includes an sdAb dimer may also comprise a step of isolating or purifying the antigen-binding molecule. This can be done according to suitable methods known to those skilled in the art.

### 6.4. Pharmaceutical Compositions

The engineered antigen-binding molecules of the disclosure may be in the form of compositions comprising the engineered antigen-binding molecules and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the engineered antigen-binding molecules and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically or locally. The most suitable route for administration in any given case will depend on the particular antibody, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of engineered antigen-binding molecule per dose. The quantity of the engineered antigen-binding molecule in a unit dose will depend on the disease being treated, as well as other factors as are well known in the art. Such unit dosages may be in the form of a lyophilized dry powder containing an amount of the engineered antigen-binding molecule suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of the engineered antigen-binding molecule suitable for a single administration.

The pharmaceutical compositions may also be supplied in bulk from containing quantities of the engineered antigen-binding molecule suitable for multiple administrations.

When formulated into a single formulation, the engineered antigen-binding molecule can be used in approximately equimolar quantities.

Pharmaceutical compositions may be prepared for storage as lyophilized formulations or aqueous solutions by mixing an engineered antigen-binding molecule having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10% w/w of engineered antigen-binding molecule.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), and pluronic polyols. Non-ionic surfactants may be present in a range of about 0.05 mg/ml to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

### 6.5. Therapeutic Indications and Methods of Treatment

The present disclosure provides methods for using and applications for the engineered antigen-binding molecules of the disclosure.

In some embodiments, engineered antigen-binding molecules of the present disclosure may be receptor agonists with enhanced agonistic activity relative to parental antigen-binding molecules that lack the engineered cysteine residues in the sdAb variable domains. Engineered antigen-binding molecules of the disclosure may be useful in treating disease states where stimulation of a cell membrane receptor may be beneficial, including, for example, conditions where an enhanced receptor response is desirable. These may include disease states where the host has a defect related to signaling through a receptor, such as a deficiency in the receptor or receptor ligand. Deficiencies in the receptor ligand may include a lower-than-normal concentration of the receptor ligand or a mutation in the ligand that makes it less effective at stimulating signaling through the receptor. Other therapeutic uses for the engineered antigen-binding molecules of the present disclosure may include enhancing immune activation by agonizing stimulatory immune receptors and/or cytokine receptors.

In one aspect, engineered antigen-binding molecules of the disclosure for use as a medicament are provided. In further aspects, engineered antigen-binding molecules of the disclosure for use in treating a disease or condition are provided. In certain embodiments, engineered antigen-binding molecules of the disclosure for use in a method of treatment are provided. In one embodiment, the disclosure provides engineered antigen-binding molecules as described herein for use in the treatment of a disease or condition in a subject in need thereof. In certain embodiments, the disclosure provides engineered antigen-binding molecules for use in a method of treating a subject having a disease or condition comprising administering to the individual a therapeutically effective amount of the engineered antigen-binding molecule. In certain embodiments the method further comprises administering to the individual a therapeutically effective amount of at least one additional therapeutic agent. In further embodiments, the disclosure provides an engineered antigen-binding molecule for use in stimulating the immune system. In certain embodiments, the disclosure provides an engineered antigen-binding molecule for use in a method of stimulating the immune system in a subject comprising administering to the individual an effective amount of an engineered antigen-binding molecule to stimulate the immune system. An “individual” according to any of the above embodiments may be a mammal, preferably a human. “Stimulation of the immune system” according to any of the above embodiments may include any one or more of a general increase in immune function, an increase in T-cell function, an increase in B cell function, a restoration of lymphocyte function, an increase in the expression of IL2 receptors, an increase in T-cell responsiveness, an increase in natural killer cell activity or lymphokine-activated killer (LAK) cell activity, and the like.

In a further aspect, the disclosure provides for the use of an engineered antigen-binding molecule of the disclosure in the manufacture or preparation of a medicament for the treatment of a disease or condition in a subject in need thereof. In one embodiment, the medicament is for use in a method of treating a disease or condition comprising administering to a subject having the disease or condition a therapeutically effective amount of the medicament. In one such embodiment, the method further comprises administering to the individual a therapeutically effective amount of at least one additional therapeutic agent. In a further embodiment, the medicament is for stimulating the immune system. In a further embodiment, the medicament is for use in a method of stimulating the immune system in a subject comprising administering to the individual an amount effective of the medicament to stimulate the immune system. In certain embodiments the disease to be treated is a proliferative disorder such as, for example, cancer. In one such embodiment, the method further comprises administering to the individual a therapeutically effective amount of at least one additional therapeutic agent, e.g., an anti-cancer agent if the disease to be treated is cancer. In a further embodiment, the medicament is for stimulating the immune system. In a further embodiment, the medicament is for use in a method of stimulating the immune system in a subject comprising administering to the individual an amount effective of the medicament to stimulate the immune system. An “individual” according to any of the above embodiments may be a mammal, preferably a human. “Stimulation of the immune system” according to any of the above embodiments may include any one or more of a general increase in immune function, an increase in T-cell function, an increase in B cell function, a restoration of lymphocyte function, an increase in the expression of IL2 receptors or other immunostimulatory receptors, an increase in T-cell responsiveness, an increase in natural killer cell activity or lymphokine-activated killer (LAK) cell activity, and the like.

Further provided are methods of agonizing a target molecule such as, for example, a membrane receptor, by contacting the target molecule with an engineered antigen-binding molecule of the disclosure. The target molecule may be present in or on the surface of a cultured cell or in or on the surface of a cell within an individual. The target molecule to be agonized in such methods may include any of the target molecules disclosed herein.

An “individual” according to any of the above embodiments may be a mammal, preferably a human. “Stimulation of the immune system” according to any of the above embodiments may include any one or more of a general increase in immune function, an increase in T-cell function, an increase in B cell function, a restoration of lymphocyte function, an increase in the expression of IL2 receptors or other immunostimulatory receptors, an increase in T-cell responsiveness, an increase in natural killer cell activity or lymphokine-activated killer (LAK) cell activity, and the like.

Engineered antigen-binding molecules of the disclosure, in particular those that target TNF family receptors such as, for example, CD40, OX40, GITR, 4-1BB, CD27, HVEM, or CD30, may be useful in treating disease states where stimulation of the immune system of the host is beneficial, in particular conditions where an enhanced cellular immune response is desirable. These may include disease states where the host immune response is insufficient or deficient.

Disease states for which the engineered antigen-binding molecules of the disclosure can be administered comprise, for example, a tumor or infection where a cellular immune response would be a critical mechanism for specific immunity. Specific disease states for which engineered antigen-binding molecules of the present disclosure can be employed include cancer, including breast cancer, prostate cancer, and colorectal cancer. The engineered antigen-binding molecules of the disclosure may be administered per se or in any suitable pharmaceutical composition.

In various embodiments, the engineered antigen-binding molecules of the disclosure are useful for the treatment of cancer, for the prevention or treatment of metastasis, for stimulating the formation, stability and/or activity of a cytotoxic immune synapse, for inducing tumor cytolysis, for inducing anti-tumor cytotoxicity, for stimulating an immune response against a tumor, or any combination of two or more of the foregoing uses.

In a further aspect, the disclosure provides a method for treating a disease in a subject, comprising administering to said individual a therapeutically effective amount of an engineered antigen-binding molecule of the disclosure. In one embodiment, a composition comprising an engineered antigen-binding molecule in a pharmaceutically acceptable form is administered to said individual. In certain embodiments the disease to be treated by administering engineered antigen-binding molecules of the present disclosure is a proliferative disorder. In some embodiments, the disease is cancer. In certain embodiments the method further comprises administering to the individual a therapeutically effective amount of at least one additional therapeutic agent, e.g., an anti-cancer agent if the disease to be treated is cancer. In further embodiments, the disclosure provides a tumor-targeted engineered antigen-binding molecule for use in stimulating the immune system. In certain embodiments, the disclosure provides a tumor-targeted engineered antigen-binding molecule for use in a method of stimulating the immune system in a subject comprising administering to the individual an effective amount of the tumor-targeted engineered antigen-binding molecule to stimulate the immune system.

In certain embodiments the disease to be treated is a proliferative disorder, preferably cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that can be treated using an engineered antigen-binding molecule of the present disclosure include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. Similarly, other cell proliferation disorders can also be treated by the engineered antigen-binding molecules of the present disclosure. Examples of such cell proliferation disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other cell proliferation disease, besides neoplasia, located in an organ system listed above.

The present disclosure further provides a method of localized delivery of an engineered antigen-binding molecule, comprising administering to a subject an engineered antigen-binding molecule as described herein, where engineered antigen-binding molecule comprises a targeting moiety that recognizes a target molecule that is expressed by a tissue to which the engineered antigen-binding molecule is to be locally delivered. As used herein, the term “locally delivered” does not require local administration but rather indicates that the engineered antigen-binding molecule be selectively localized to a tissue of interest following administration. In some embodiments, the administration is not local to the tissue.

A skilled artisan readily recognizes that in many cases the engineered antigen-binding molecule may not provide a cure but may only provide partial benefit. In some embodiments, a physiological change having some benefit is also considered therapeutically beneficial. Thus, in some embodiments, an amount of an engineered antigen-binding molecule that provides a physiological change is considered an “effective amount” or a “therapeutically effective amount.”

The subject, patient, or individual in need of treatment is typically a mammal, more specifically a human.

The appropriate dosage of an engineered antigen-binding molecule of the disclosure (when used alone or in combination with one or more other additional therapeutic agents) will depend on the type of disease to be treated, the route of administration, the body weight of the patient, the particular engineered antigen-binding molecule, the severity and course of the disease, whether the antibody is administered for preventive or therapeutic purposes, previous or concurrent therapeutic interventions, the patient's clinical history and response to the engineered antigen-binding molecule, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

A therapeutically effective amount of an engineered antigen-binding molecule may comprise only a single administration or many administrations over a period of time. Thus, an engineered antigen-binding molecule is suitably administered to the patient at one time or over a series of treatments. Depending on the type and severity of the disease, about 1 μg/kg to 15 mg/kg (e.g., 0.1 mg/kg-10 mg/kg) of an engineered antigen-binding molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of an engineered antigen-binding molecule would be in the range from about 0.005 mg/kg to about 10 mg/kg. In other non-limiting examples, a dose may also comprise from about 1 μg/kg/body weight, about 5 μg/kg/body weight, about 10 μg/kg/body weight, about 50 μg/kg/body weight, about 100 μg/kg/body weight, about 200 μg/kg/body weight, about 350 μg/kg/body weight, about 500 μg/kg/body weight, about 1 mg/kg/body weight, about 5 mg/kg/body weight, about 10 mg/kg/body weight, about 50 mg/kg/body weight, about 100 mg/kg/body weight, about 200 mg/kg/body weight, about 350 mg/kg/body weight, about 500 mg/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 μg/kg/body weight to about 500 mg/kg/body weight, etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g., every week or every three weeks (e.g., such that the patient receives from about two to about twenty, or e.g., about six doses of the engineered antigen-binding molecule). An initial higher loading dose, followed by one or more lower doses may be administered. However, other dosage regimens may be useful. The progress of this therapy is easily monitored by conventional techniques and assays.

For systemic administration, a therapeutically effective dose can be estimated initially from in vitro assays, such as cell culture assays. A dose can then be formulated in animal models to achieve a circulating concentration range that includes the EC50 as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the engineered antigen-binding molecule which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by ELISA HPLC.

In cases of local administration or selective uptake, the effective local concentration of the engineered antigen-binding molecule may not be related to plasma concentration. One having skill in the art will be able to optimize therapeutically effective local dosages without undue experimentation.

### 6.6. Combination Therapy

The engineered antigen-binding molecules disclosed herein may be administered in combination with one or more other agents in therapy. For instance, an engineered antigen-binding molecule of the disclosure may be co-administered with at least one additional therapeutic agent. The term “therapeutic agent” encompasses any agent administered to treat a symptom or disease in a subject in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of engineered antigen-binding molecule used, the type of disorder or treatment, and other factors discussed above. The engineered antigen-binding molecules are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the engineered antigen-binding molecule can occur prior to, simultaneously, and/or following, administration of the additional therapeutic agent and/or adjuvant.

### 7. SPECIFIC EMBODIMENTS

The present disclosure is exemplified by the specific numbered embodiments below.
1. An antigen-binding molecule comprising:
   (a) a first polypeptide chain comprising:
      (i) a first single domain antibody (sdAb) variable domain comprising a first cysteine residue; and
      (ii) a first Fc domain; and
   (b) a second polypeptide chain comprising:
      (i) a second sdAb variable domain comprising a second cysteine residue; and
      (ii) a second Fc domain associated with the first Fc domain;
   optionally wherein the first cysteine residue and the second cysteine residue are substitutions of non-cysteine amino acids in a reference sdAb sequence.
2. The antigen-binding molecule of embodiment 1, wherein the first cysteine residue is at a position in the first sdAb variable domain that is different from the position of the second cysteine residue in the second sdAb variable domain.
3. The antigen-binding molecule of embodiment 1 or 2, wherein the first sdAb variable domain comprises a framework region 1 (FR1), a framework region 2 (FR2), a framework region 3 (FR3), and a framework region 4 (FR4).
4. The antigen-binding molecule of any one of embodiments 1 to 3, wherein the second sdAb variable domain comprises an FR1, an FR2, an FR3, and an FR4.
5. The antigen-binding molecule of embodiment 3 or 4, wherein the first cysteine residue is in FR4 of the first sdAb variable domain.
6. The antigen-binding molecule of any one of embodiments 3 to 5, wherein the second cysteine residue is in FR2 of the second sdAb variable domain.
7. The antigen-binding molecule of any one of embodiments 1 to 6, wherein the first sdAb variable domain further comprises a third cysteine residue, wherein the third cysteine residue is a substitution of a non-cysteine amino acid in a reference sdAb sequence and is at the same relative position in the first sdAb variable domain as the position of the second cysteine in the second sdAb variable domain.
8. The antigen-binding molecule of any one of embodiments 1 to 7, wherein the second sdAb variable domain further comprises a fourth cysteine residue, wherein the fourth cysteine residue is a substitution of a non-cysteine amino acid in a reference sdAb sequence and is at the same relative position in the second sdAb variable domain as the position of the first cysteine in the first sdAb variable domain.
9. The antigen-binding molecule of embodiment 7 or 8, further comprising: (A) a first intra-chain disulfide bond between the first cysteine residue and the third cysteine residue; and (B) a second intra-chain disulfide bond between the second cysteine residue and the fourth cysteine residue.
10. The antigen-binding molecule of embodiment 9, wherein the first sdAb and the second sdAb have a stronger inter-chain association as compared to a reference pair of sdAbs lacking the first, second, third and/or fourth cysteine residues.
11. The antigen-binding molecule of any one of embodiments 4 to 10, wherein the FR4 of the first sdAb variable domain associates with the second sdAb variable domain and wherein the FR4 of the second sdAb variable domain associates with the first sdAb variable domain.
12. The antigen-binding molecule of any one of embodiments 9 to 11, wherein the first cysteine residue is at a position corresponding to W106 of a human VH, numbering according to Kabat.
13. The antigen-binding molecule of any one of embodiments 9 to 12, wherein the second cysteine residue is at a position corresponding to L45 of a human VH, numbering according to Kabat.
14. The antigen-binding molecule of any one of embodiments 9 to 13, wherein the first sdAb variable domain is a first human sdVH and the second sdAb variable domain is a second human sdVH.
15. The antigen-binding molecule of embodiment 14, wherein the first cysteine residue is at position W106 of the first human sdVH and the second cysteine residue is at position L45 of the second human sdVH, numbering according to Kabat.
16. The antigen-binding molecule of embodiment 14 or 15, wherein the third cysteine residue is at position L45 of the first human sdVH and the fourth cysteine residue is at position W106 of the second human sdVH, numbering according to Kabat.
17. The antigen-binding molecule of any one of embodiments 9 to 13, wherein the first sdAb variable domain is a first VHH and the second sdAb variable domain is a second VHH.
18. The antigen-binding molecule of embodiment 17, wherein the first cysteine residue is at position R106 of the first VHH and the second cysteine residue is at position R45 of the second VHH, numbering according to Kabat.
19. The antigen-binding molecule of embodiment 17 or 18, wherein the third cysteine residue is at position R45 of the first VHH and the fourth cysteine residue is at position R106 of the second VHH, numbering according to Kabat.
20. The antigen-binding molecule of any one of embodiments 1 to 8, further comprising an inter-chain disulfide bond between the first cysteine residue and the second cysteine residue.
21. The antigen-binding molecule of embodiment 20, wherein the first cysteine residue is at a position corresponding to Q108 of a human VH, numbering according to Kabat.
22. The antigen-binding molecule of embodiment 20 or 21, wherein the second cysteine residue is at a position corresponding to G44 of a human VH, numbering according to Kabat.
23. The antigen-binding molecule of any one of embodiments 20 to 22, wherein the first sdAb variable domain is a first human sdVH and the second sdAb variable domain is a second human sdVH.
24. The antigen-binding molecule of embodiment 23, wherein the first cysteine residue is at position Q108 of the first human sdVH and the second cysteine residue is at position G44 of the second human sdVH, numbering according to Kabat.
25. The antigen-binding molecule of embodiment 22 or 23, wherein the third cysteine residue is at position G44 of the first human sdVH and the fourth cysteine residue is at position Q108 of the second human sdVH, numbering according to Kabat.
26. The antigen-binding molecule of any one of embodiments 20 to 22, wherein the first sdAb variable domain is a first VHH and the second sdAb variable domain is a second VHH.
27. The antigen-binding molecule of embodiment 26, wherein the first cysteine residue is at position Q108 of the first VHH and the second cysteine residue is at position E44 of the second VHH, numbering according to Kabat.
28. The antigen-binding molecule of embodiment 26 or 27, wherein the third cysteine residue is at position E44 of the first VHH and the fourth cysteine residue is at position Q108 of the second VHH, numbering according to Kabat.
29. An antigen-binding molecule comprising:
   (a) a first polypeptide chain comprising:
      (i) a first single domain antibody (sdAb) variable domain comprising a first cysteine residue; and
      (ii) a first Fc domain; and
   (b) a second polypeptide chain comprising:
      (i) a second sdAb variable domain comprising a second cysteine residue covalently bonded to the first cysteine residue; and
      (ii) a second Fc domain associated with the first Fc domain.
30. The antigen-binding molecule of embodiment 29, wherein the first cysteine residue and the second cysteine residue form a disulfide bond.
31. The antigen-binding molecule of embodiment 29 or 30, wherein the first cysteine residue and the second cysteine residue are substitutions of non-cysteine amino acids in a reference sdAb sequence.
32. The antigen-binding molecule any one of embodiments 29 to 31, wherein the first sdAb variable domain comprises an FR1, an FR2, an FR3, and an FR4 and the second sdAb variable domain the second sdAb variable domain comprises an FR1, an FR2, an FR3, and an FR4.
33. The antigen-binding molecule of embodiment 32, wherein the first cysteine residue is in FR4 of the first sdAb variable domain.
34. The antigen-binding molecule of embodiment 32 or 33, wherein the second cysteine residue is in FR2 of the second sdAb variable domain.
35. The antigen-binding molecule of any one of embodiments 29 to 34, wherein the first sdAb variable domain further comprises a third cysteine residue, wherein the third cysteine residue is a substitution of a non-cysteine amino acid in a reference sdAb sequence and is at the same relative position in the first sdAb variable domain as the position of the second cysteine in the second sdAb variable domain.
36. The antigen-binding molecule of any one of embodiments 29 to 35, wherein the second sdAb variable domain further comprises a fourth cysteine residue, wherein the fourth cysteine residue is a substitution of a non-cysteine amino acid in a reference sdAb sequence and is at the same relative position in the second sdAb variable domain as the position of the first cysteine in the first sdAb variable domain.
37. The antigen-binding molecule of any one of embodiments 29 to 36, wherein the first cysteine residue is at a position corresponding to Q108 of a human VH, numbering according to Kabat.
38. The antigen-binding molecule of any one of embodiments 29 to 37, wherein the second cysteine residue is at a position corresponding to G44 of a human VH, numbering according to Kabat.
39. The antigen-binding molecule of any one of embodiments 29 to 38, wherein the first sdAb variable domain is a first human sdVH and the second sdAb variable domain is a second human sdVH.
40. The antigen-binding molecule of embodiment 39, wherein the first cysteine residue is at position Q108 of the first human sdVH and the second cysteine residue is at position G44 of the second human sdVH, numbering according to Kabat.
41. The antigen-binding molecule of embodiment 39 or 40, wherein the third cysteine residue is at position G44 of the first human sdVH and the fourth cysteine residue is at position Q108 of the second human sdVH, numbering according to Kabat.
42. The antigen-binding molecule of any one of embodiments 29 to 38, wherein the first sdAb variable domain is a first VHH and the second sdAb variable domain is a second VHH.
43. The antigen-binding molecule of embodiment 42, wherein the first cysteine residue is at position Q108 of the first VHH and the second cysteine residue is at position E44 of the second VHH, numbering according to Kabat.
44. The antigen-binding molecule of embodiment 42 or 43, wherein the third cysteine residue is at position E44 of the first VHH and the fourth cysteine residue is at position Q108 of the second VHH, numbering according to Kabat.
45. A antigen-binding molecule comprising:
   (a) a first polypeptide chain comprising a first sdAb variable domain comprising an FR1, an FR2, an FR3, and an FR4, wherein the FR4 comprises a first cysteine residue, optionally wherein the first cysteine residue is a substitution of a non-cysteine amino acid in a reference FR4 sequence; and
   (b) a second polypeptide chain comprising a second sdAb variable domain comprising an FR1, an FR2, an FR3, and an FR4, wherein the FR2 comprises a second cysteine residue, optionally wherein the second cysteine residue is a substitution of a non-cysteine amino acid in a reference FR2 sequence.
46. The antigen-binding molecule of embodiment 45, wherein the first sdAb variable domain further comprises a third cysteine residue, wherein the third cysteine residue is a substitution of a non-cysteine amino acid in a reference sdAb sequence and is at the same relative position in the first sdAb variable domain as the position of the second cysteine in the second sdAb variable domain.
47. The antigen-binding molecule of embodiment 45 or 46, wherein the second sdAb variable domain further comprises a fourth cysteine residue, wherein the fourth cysteine residue is a substitution of a non-cysteine amino acid in a reference sdAb sequence and is at the same relative position in the second sdAb variable domain as the position of the first cysteine in the first sdAb variable domain.
48. The antigen-binding molecule of embodiment 46 or 47, further comprising: (A) a first intra-chain disulfide bond between the first cysteine residue and the third cysteine residue; and (B) a second intra-chain disulfide bond between the second cysteine residue and the fourth cysteine residue.
49. The antigen-binding molecule of embodiment 48, wherein the first sdAb and the second sdAb have a stronger inter-chain association as compared to a reference pair of sdAbs lacking the first, second, third and/or fourth cysteine residues.
50. The antigen-binding molecule of embodiment 48 or 49, wherein the FR4 of the first sdAb variable domain associates with the second sdAb variable domain and wherein the FR4 of the second sdAb variable domain associates with the first sdAb variable domain.
51. The antigen-binding molecule of any one of embodiments 48 to 50, wherein the first cysteine residue is at a position corresponding to W106 of a human VH, numbering according to Kabat.
52. The antigen-binding molecule of any one of embodiments 48 to 51, wherein the second cysteine residue is at a position corresponding to L45 of a human VH, numbering according to Kabat.
53. The antigen-binding molecule of any one of embodiments 48 to 52, wherein the first sdAb variable domain is a first human sdVH and the second sdAb variable domain is a second human sdVH.
54. The antigen-binding molecule of embodiment 53, wherein the first cysteine residue is at position W106 of the first human sdVH and the second cysteine residue is at position L45 of the second human sdVH, numbering according to Kabat.
55. The antigen-binding molecule of embodiment 53 or 54, wherein the third cysteine residue is at position L45 of the first human sdVH and the fourth cysteine residue is at position W106 of the second human sdVH, numbering according to Kabat.
56. The antigen-binding molecule of any one of embodiments 48 to 52, wherein the first sdAb variable domain is a first VHH and the second sdAb variable domain is a second VHH.
57. The antigen-binding molecule of embodiment 56, wherein the first cysteine residue is at position R106 of the first VHH and the second cysteine residue is at position R45 of the second VHH, numbering according to Kabat.
58. The antigen-binding molecule of embodiment 56 or 57, wherein the third cysteine residue is at position R45 of the first VHH and the fourth cysteine residue is at position R106 of the second VHH, numbering according to Kabat.
59. The antigen-binding molecule of any one of embodiments 45 to 47, further comprising an inter-chain disulfide bond between the first cysteine residue and the second cysteine residue.
60. The antigen-binding molecule of embodiment 59, wherein the first cysteine residue is at a position corresponding to Q108 of a human VH, numbering according to Kabat.
61. The antigen-binding molecule of embodiment 58 or 59, wherein the second cysteine residue is at a position corresponding to G44 of a human VH, numbering according to Kabat.
62. The antigen-binding molecule of any one of embodiments 58 to 61, wherein the first sdAb variable domain is a first human sdVH and the second sdAb variable domain is a second human sdVH.
63. The antigen-binding molecule of embodiment 62, wherein the first cysteine residue is at position Q108 of the first human sdVH and the second cysteine residue is at position G44 of the second human sdVH, numbering according to Kabat.
64. The antigen-binding molecule of embodiment 62 or 63, wherein the third cysteine residue is at position G44 of the first human sdVH and the fourth cysteine residue is at position Q108 of the second human sdVH, numbering according to Kabat.
65. The antigen-binding molecule of any one of embodiments 58 to 61, wherein the first sdAb variable domain is a first VHH and the second sdAb variable domain is a second VHH.
66. The antigen-binding molecule of embodiment 65, wherein the first cysteine residue is at position Q108 of the first VHH and the second cysteine residue is at position E44 of the second VHH, numbering according to Kabat.
67. The antigen-binding molecule of embodiment 65 or 66, wherein the third cysteine residue is at position E44 of the first VHH and the fourth cysteine residue is at position Q108 of the second VHH, numbering according to Kabat.
68. An antigen-binding molecule comprising:
   (a) a first polypeptide chain comprising a first sdAb variable domain comprising an FR1, an FR2, an FR3, and an FR4, wherein the FR4 comprises a first cysteine residue; and
   (b) a second polypeptide chain comprising a second sdAb variable domain comprising an FR1, an FR2, an FR3, and an FR4, wherein the FR2 comprises a second cysteine residue, wherein the second cysteine residue is covalently bonded to the first cysteine residue.
69. The antigen-binding molecule of embodiment 68, wherein the first cysteine residue and the second cysteine residue form a disulfide bond.
70. The antigen-binding molecule of embodiment 68 or 69, wherein the first cysteine residue and the second cysteine residue are substitutions of non-cysteine amino acids in a reference sdAb sequence.
71. The antigen-binding molecule of any one of embodiments 68 to 70, wherein the first sdAb variable domain further comprises a third cysteine residue, wherein the third cysteine residue is a substitution of a non-cysteine amino acid in a reference sdAb sequence and is at the same relative position in the first sdAb variable domain as the position of the second cysteine in the second sdAb variable domain.
72. The antigen-binding molecule of any one of embodiments 68 to 71, wherein the second sdAb variable domain further comprises a fourth cysteine residue, wherein the fourth cysteine residue is a substitution of a non-cysteine amino acid in a reference sdAb sequence and is at the same relative position in the second sdAb variable domain as the position of the first cysteine in the first sdAb variable domain.
73. The antigen-binding molecule of any one of embodiments 68 to 72, wherein the first cysteine residue is at a position corresponding to Q108 of a human VH, numbering according to Kabat.
74. The antigen-binding molecule of any one of embodiments 68 to 73, wherein the second cysteine residue is at a position corresponding to G44 of a human VH, numbering according to Kabat.
75. The antigen-binding molecule of any one of embodiments 68 to 74, wherein the first sdAb variable domain is a first human sdVH and the second sdAb variable domain is a second human sdVH.
76. The antigen-binding molecule of embodiment 75, wherein the first cysteine residue is at position Q108 of the first human sdVH and the second cysteine residue is at position G44 of the second human sdVH, numbering according to Kabat.
77. The antigen-binding molecule of embodiment 75 or 76, wherein the third cysteine residue is at position G44 of the first human sdVH and the fourth cysteine residue is at position Q108 of the second human sdVH, numbering according to Kabat.
78. The antigen-binding molecule of any one of embodiments 68 to 74, wherein the first sdAb variable domain is a first VHH and the second sdAb variable domain is a second VHH.
79. The antigen-binding molecule of embodiment 78, wherein the first cysteine residue is at position Q108 of the first VHH and the second cysteine residue is at position E44 of the second VHH, numbering according to Kabat.
80. The antigen-binding molecule of embodiment 78 or 79, wherein the third cysteine residue is at position E44 of the first VHH and the fourth cysteine residue is at position Q108 of the second VHH, numbering according to Kabat.
81. The antigen-binding molecule of any one of embodiments 45 to 80, wherein the first polypeptide chain further comprises a first dimerization moiety.
82. The antigen-binding molecule of any one of embodiments 45 to 81, wherein the second polypeptide chain further comprises a second dimerization moiety.
83. The antigen-binding molecule of any one of embodiments 81 or 82, wherein the first dimerization moiety comprises a first Fc domain and the second dimerization moiety comprises a second Fc domain.
84. The antigen-binding molecule of any one of embodiments 1 to 44 or 81 to 83, wherein the first Fc domain and the second Fc domain are IgG Fc domains.
85. The antigen-binding molecule of any one of embodiments 1 to 44 or 81 to 84, wherein the first Fc domain and the second Fc domain are IgG1 Fc domains.
86. The antigen-binding molecule of any one of embodiments 1 to 44 or 81 to 85, wherein the first Fc domain comprises a first IgG hinge domain, a first IgG CH2 domain, and a first IgG CH3 domain.
87. The antigen-binding molecule of any one of embodiments 1 to 44 or 81 to 86, wherein the second Fc domain comprises a second IgG hinge domain, a second IgG CH2 domain, and a second IgG CH3 domain.
88. The antigen-binding molecule of embodiment 86 or 87, wherein the first IgG hinge domain and the second IgG hinge domain are joined via one or more disulfide bonds.
89. The antigen-binding molecule of any one of embodiments 1 to 44 or 81 to 88, wherein the first Fc domain and the second Fc domain have the same amino acid sequence.
90. The antigen-binding molecule of any one of embodiments 86 to 89, wherein the first IgG CH2 domain and/or the second IgG CH2 domain comprises one or more amino acid substitutions that reduce binding to an Fc receptor.
91. The antigen-binding molecule of embodiment 90, wherein the Fc receptor is an Fcγ receptor.
92. The antigen-binding molecule of embodiment 90 or 91, wherein the Fc receptor is FcγRIIIa.
93. The antigen-binding molecule of any one of embodiments 86 to 92, wherein the first IgG CH2 domain and/or the second IgG CH2 domain comprises one or more amino acid substitutions that reduce effector function.
94. The antigen-binding molecule of any one of embodiments 86 to 93, wherein the first IgG CH2 domain and/or the second IgG CH2 domain comprise one or more amino acid substitutions that increase binding to an Fc receptor.
95. The antigen-binding molecule of embodiment 94, wherein the Fc receptor is FcγRIIB.
96. The antigen-binding molecule of any one of embodiments 86 to 95, wherein the first IgG CH3 domain and/or the second IgG CH3 domain comprise one or more amino acid substitutions that reduce binding to an Fc receptor.
97. The antigen-binding molecule of embodiment 96, wherein the Fc receptor is an Fcγ receptor.
98. The antigen-binding molecule of embodiment 96 or 97, wherein the Fc receptor is FcγRIIIa.
99. The antigen-binding molecule of any one of embodiments 86 to 98, wherein the first IgG CH3 domain and/or the second IgG CH3 domain comprise one or more amino acid substitutions that reduce effector function.
100. The antigen-binding molecule of any one of embodiments 86 to 99, wherein the first IgG CH3 domain and/or the second IgG CH3 domain comprises one or more amino acid substitutions that increase binding to an Fc receptor.
101. The antigen-binding molecule of embodiment 100, wherein the Fc receptor is FcγRIIB.
102. The antigen-binding molecule of any one of embodiments 86 to 101, wherein (a) the first IgG CH2 and the first IgG CH3 domain, and/or (b) the second IgG CH2 domain and the second IgG CH3 domain together comprise an amino acid sequence having at least 90% sequence identity to an amino acid sequence set forth in Table F-1.
103. The antigen-binding molecule of any one of embodiments 86 to 102, wherein the first polypeptide chain comprises, from N- to C-terminus:
   (a) the first sdAb variable domain;
   (b) the first IgG hinge domain;
   (c) the first IgG CH2 domain; and
   (d) the first IgG CH3 domain.
104. The antigen-binding molecule of any one of embodiments 86 to 103, wherein the second polypeptide chain comprises, from N- to C-terminus:
   (a) the second sdAb variable domain;
   (b) the second IgG hinge domain;
   (c) the second IgG CH2 domain; and
   (d) the second IgG CH3 domain.
105. The antigen-binding molecule of any one of embodiments 1 to 104, wherein the antigen-binding molecule is monospecific.
106. The antigen-binding molecule of any one of embodiments 1 to 104, wherein the first sdAb variable domain and the second sdAb variable domain each have specific affinity for a target molecule.
107. The antigen-binding molecule of embodiment 105, wherein the target molecule is a component of a membrane-bound receptor.
108. The antigen-binding molecule of embodiment 107, wherein the membrane-bound receptor comprises multiple copies of the target molecule.
109. The antigen-binding molecule of any one of embodiments 105 to 108, wherein the target molecule is a TNF family receptor.
110. The antigen-binding molecule of embodiment 109, wherein the TNF family receptor is CD40, OX40, GITR, 4-1BB, CD27, HVEM, or CD30.
111. The antigen-binding molecule of any one of embodiments 105 to 110, wherein the target molecule is OX40, the first sdAb variable domain is a first VHH, and the second sdAb variable domain is a second VHH.
112. The antigen-binding molecule of embodiment 111, wherein the first VHH comprises a Q108C amino acid substitution, numbering according to Kabat, and an amino acid sequence having at least 90% sequence identity to the amino acid sequence of SEQ ID NO: 36, and the second VHH comprises an E44C amino acid substitution, numbering according to Kabat, and an amino acid sequence having at least 90% sequence identity to the amino acid sequence of SEQ ID NO:36.
113. The antigen-binding molecule of embodiment 111 or 112, wherein the first polypeptide chain comprises a Q108C amino acid substitution in the first VHH, numbering according to Kabat, and an amino acid sequence having at least 90% sequence identity to the amino acid sequence of SEQ ID NO:35 and the second polypeptide chain comprises an E44C amino acid substitution in the second VHH, numbering according to Kabat, and an amino acid sequence having at least 90% sequence identity to the amino acid sequence of SEQ ID NO:35.
114. The antigen-binding molecule of embodiment 111, wherein the first VHH comprises an R45C and an R106C amino acid substitution, numbering according to Kabat, and amino acid sequence having at least 90% sequence identity to the amino acid sequence of SEQ ID NO:38, and the second VHH comprises an R45C and an R106C amino acid substitution, numbering according to Kabat, and amino acid sequence having at least 90% sequence identity to the amino acid sequence of SEQ ID NO:38.
115. The antigen-binding molecule of embodiment 111 or 114, wherein the first polypeptide chain comprises an R45C and an R106C amino acid substitution in the first VHH and amino acid sequence having at least 90% sequence identity to the amino acid sequence of SEQ ID NO:37, and the second polypeptide chain comprises an R45C and an R106C amino acid substitution in the second VHH and amino acid sequence having at least 90% sequence identity to the amino acid sequence of SEQ ID NO:37.
116. The antigen-binding molecule of any one of embodiments 105 to 108, wherein the target molecule is a homodimeric class I cytokine receptor.
117. The antigen-binding molecule of embodiment 116, wherein the homodimeric class I cytokine receptor is EPO receptor, G-CSF receptor, leptin receptor (LEPR), thrombopoietin receptor (TPOR), growth hormone receptor (GHR), or prolactin receptor (PRLR).
118. The antigen-binding molecule of any one of embodiments 1 to 108, wherein the first sdAb and the second sdAb comprise the sequence of any of the sdAbs set forth in Table B, except that the first sdAb comprises the first cysteine residue and the second sdAb comprises the second cysteine residue.
119. The antigen-binding molecule of embodiment 118, wherein the first sdAb further comprises the third cysteine residue and the second sdAb further comprises the fourth cysteine residue.
120. The antigen-binding molecule of embodiment 118 or 119, wherein the complete amino acid sequence of the first sdAb is identical to the complete amino acid sequence of the second sdAb.
121. A host cell engineered to express the antigen-binding molecule of any one of embodiments 1 to 120.
122. A host cell transfected with one or more expression vectors comprising one or more nucleic acid sequences encoding the antigen-binding molecule of any one of embodiments 1 to 120 under the control of one or more promoters.
123. A method of producing the antigen-binding molecule of any one of embodiments 1 to 120, comprising culturing the host cell of embodiment 121 or 122 and recovering the antigen-binding molecule expressed thereby.
124. A pharmaceutical composition comprising the antigen-binding molecule of any one of embodiments 1 to 120 and an excipient.
125. A method of agonizing a target molecule comprising contacting the target molecule with the molecule of any one of embodiments 1 to 120 in which the first and/or second sdAb bind to the target molecule.
126. The method of embodiment 125, wherein the method is performed in vivo.
127. The method of embodiment 126, wherein the method comprises administering the antigen-binding molecule to a subject in need thereof.
128. The method of embodiment 125, wherein the method is performed in vitro or ex vivo.
129. The method of any one of embodiments 125 to 128, wherein the first sdAb and the second sdAb are each agonist sdAbs.
130. A method of activating an immune response in a subject in need thereof, the method comprising administering to the subject the antigen-binding molecule of any one of embodiments 1 to 120.
131. The method of embodiment 130, wherein the subject has cancer.
132. The method of embodiment 130, wherein the subject is at risk of developing cancer.
133. The method of any one of embodiments 130 to 132, wherein the antigen-binding molecule binds to a target molecule on an immune cell.
134. The method of embodiment 133, wherein the immune cell is a T cell.
135. A method comprising administering to a subject in need thereof the antigen-binding molecule of any one of embodiments 1 to 120.
136. The method of embodiment 135, which is a method for the treatment of cancer.
137. The method of embodiment 135 or 136, which is a method for the prevention or treatment of metastasis.
138. The method of any one of embodiments 135 to 137, which is a method for clustering a target molecule, wherein the antigen-binding molecule binds to the target molecule.
139. The method of any one of embodiments 135 to 138, which is a method for inducing tumor cytolysis.
140. The method of any one of embodiments 135 to 139, which is a method for inducing anti-tumor cytotoxicity.
141. The method of any one of embodiments 135 to 140, which is a method for stimulating an immune response against a tumor.
142. A method for forming a dimer of sdAb moieties within an antigen-binding molecule, the method comprising co-expressing a first polypeptide chain and a second polypeptide chain within a cell, wherein:
   (a) the first polypeptide chain comprises:
      (i) a first single domain antibody (sdAb) variable domain comprising a first cysteine residue; and
      (ii) a first Fc domain; and
   (b) the second polypeptide chain comprises:
      (i) a second sdAb variable domain comprising a second cysteine residue; and
      (ii) a second Fc domain;
   wherein the first Fc domain and the second Fc domain associate to form an Fc dimer; and
   wherein the first cysteine residue covalently bonds to the second cysteine residue to form an sdAb dimer.
143. The method of embodiment 142, wherein the first sdAb variable domain comprises an FR1, an FR2, an FR3, and an FR4 and the second sdAb variable domain the second sdAb variable domain comprises an FR1, an FR2, an FR3, and an FR4.
144. The method of embodiment 143, wherein the first cysteine residue is in FR4 of the first sdAb variable domain.
145. The method of embodiment 143 or 144, wherein the second cysteine residue is in FR2 of the second sdAb variable domain.
146. The method of any one of embodiments 142 to 145, wherein the first sdAb variable domain further comprises a third cysteine residue, wherein the third cysteine residue is a substitution of a non-cysteine amino acid in a reference sdAb sequence and is at the same relative position in the first sdAb variable domain as the position of the second cysteine in the second sdAb variable domain.
147. The method of any one of embodiments 142 to 146, wherein the second sdAb variable domain further comprises a fourth cysteine residue, wherein the fourth cysteine residue is a substitution of a non-cysteine amino acid in a reference sdAb sequence and is at the same relative position in the second sdAb variable domain as the position of the first cysteine in the first sdAb variable domain.
148. The method of any one of embodiments 142 to 147, wherein the first cysteine residue is at a position corresponding to Q108 of a human VH, numbering according to Kabat.
149. The method of any one of embodiments 142 to 148, wherein the second cysteine residue is at a position corresponding to G44 of a human VH, numbering according to Kabat.
150. The method of any one of embodiments 142 to 149, wherein the first sdAb variable domain is a first human sdVH and the second sdAb variable domain is a second human sdVH.
151. The method of embodiment 150, wherein the first cysteine residue is at position Q108 of the first human sdVH and the second cysteine residue is at position G44 of the second human sdVH, numbering according to Kabat.
152. The method of embodiment 150 or 151, wherein the third cysteine residue is at position G44 of the first human sdVH and the fourth cysteine residue is at position Q108 of the second human sdVH, numbering according to Kabat.
153. The method of any one of embodiments 142 to 149, wherein the first sdAb variable domain is a first VHH and the second sdAb variable domain is a second VHH.
154. The method of embodiment 153, wherein the first cysteine residue is at position Q108 of the first VHH and the second cysteine residue is at position E44 of the second VHH, numbering according to Kabat.
155. The method of embodiment 153 or 154, wherein the third cysteine residue is at position E44 of the first VHH and the fourth cysteine residue is at position Q108 of the second VHH, numbering according to Kabat.
156. A method for forming a dimer of sdAb moieties within an antigen-binding molecule, the method comprising co-expressing a first polypeptide chain and a second polypeptide chain within a cell, wherein:
   (a) the first polypeptide chain comprises a first sdAb variable domain comprising an FR1, an FR2, an FR3, and an FR4, wherein
      (i) the FR4 comprises a first cysteine residue, which is a substitution of a non-cysteine amino acid in a reference FR4 sequence; and
      (ii) a third cysteine residue, which is a substitution of a non-cysteine amino acid in a reference FR2 sequence; and
   (b) the second polypeptide chain comprises a second sdAb variable domain comprising an FR1, an FR2, an FR3, and an FR4, wherein:
      (i) the FR2 comprises a second cysteine residue, which is a substitution of a non-cysteine amino acid in a reference FR2 sequence; and
      (ii) a fourth cysteine residue, which is a substitution of a non-cysteine amino acid in a reference FR4 sequence;
   wherein the third cysteine residue is at the same relative position in the first sdAb variable domain as the position of the second cysteine residue in the second sdAb variable domain; and
   wherein the fourth cysteine residue is at the same relative position in the second sdAb variable domain as the position of the first cysteine residue in the first sdAb variable domain.
157. The method of embodiment 156, wherein (A) a first intra-chain disulfide bond forms between the first cysteine residue and the third cysteine residue; and (B) a second intra-chain disulfide bond forms between the second cysteine residue and the fourth cysteine residue.
158. The method of embodiment 157, wherein the first sdAb and the second sdAb have a stronger inter-chain association as compared to a reference pair of sdAbs lacking the first, second, third and/or fourth cysteine residues.
159. The method of embodiment 157 or 158, wherein the FR4 of the first sdAb variable domain associates with the second sdAb variable domain and the FR4 of the second sdAb variable domain associates with the first sdAb variable domain.
160. The method of any one of embodiments 157 to 159, wherein the first cysteine residue is at a position corresponding to W106 of a human VH, numbering according to Kabat.
161. The method of any one of embodiments 157 to 160, wherein the second cysteine residue is at a position corresponding to L45 of a human VH, numbering according to Kabat.
162. The method of any one of embodiments 157 to 161, wherein the first sdAb variable domain is a first human sdVH and the second sdAb variable domain is a second human sdVH.
163. The method of embodiment 162, wherein the first cysteine residue is at position W106 of the first human sdVH and the second cysteine residue is at position L45 of the second human sdVH, numbering according to Kabat.
164. The method of embodiment 162 or 163, wherein the third cysteine residue is at position L45 of the first human sdVH and the fourth cysteine residue is at position W106 of the second human sdVH, numbering according to Kabat.
165. The method of any one of embodiments 157 to 161, wherein the first sdAb variable domain is a first VHH and the second sdAb variable domain is a second VHH.
166. The method of embodiment 165, wherein the first cysteine residue is at position R106 of the first VHH and the second cysteine residue is at position R45 of the second VHH, numbering according to Kabat.
167. The method of embodiment 165 or 166, wherein the third cysteine residue is at position R45 of the first VHH and the fourth cysteine residue is at position R106 of the second VHH, numbering according to Kabat.
168. The method of embodiment 156, wherein an inter-chain disulfide bond forms between the first cysteine residue and the second cysteine residue.
169. The method of embodiment 168, wherein the first cysteine residue is at a position corresponding to Q108 of a human VH, numbering according to Kabat.
170. The method of embodiment 168 or 169, wherein the second cysteine residue is at a position corresponding to G44 of a human VH, numbering according to Kabat.
171. The method of any one of embodiments 168 to 170, wherein the first sdAb variable domain is a first human sdVH and the second sdAb variable domain is a second human sdVH.
172. The method of embodiment 171, wherein the first cysteine residue is at position Q108 of the first human sdVH and the second cysteine residue is at position G44 of the second human sdVH, numbering according to Kabat.
173. The method of embodiment 171 or 172, wherein the third cysteine residue is at position G44 of the first human sdVH and the fourth cysteine residue is at position Q108 of the second human sdVH, numbering according to Kabat.
174. The method of any one of embodiments 168 to 170, wherein the first sdAb variable domain is a first VHH and the second sdAb variable domain is a second VHH.
175. The method of embodiment 174, wherein the first cysteine residue is at position Q108 of the first VHH and the second cysteine residue is at position E44 of the second VHH, numbering according to Kabat.
176. The method of embodiment 174 or 175, wherein the third cysteine residue is at position E44 of the first VHH and the fourth cysteine residue is at position Q108 of the second VHH, numbering according to Kabat.
177. A method of producing a nucleic acid encoding an sdAb capable of dimerizing, the method comprising introducing into a nucleic acid encoding a parental sdAb nucleotide alterations encoding the substitutions defined in any one of embodiments 21 to 28 or 37 to 67.
178. The method of embodiment 177, wherein the sdAb is a portion of an antigen-binding molecule configured as defined in any of embodiments 1 to 120.
179. A host cell comprising a nucleic acid produced by the method of embodiment 177 or 178.
180. A method of producing the host cell of embodiment 179, which comprises:
181. A method of producing the host cell of embodiment C, which comprises:
   (a) producing a nucleic acid encoding an sdAb capable of dimerizing using the method of embodiment 177 or 178; and
   (b) introducing the nucleic acid into a parental host cell.
182. A method of generating an sdAb dimer, comprising culturing a cell line engineered to express an sdAb comprising the substitutions defined in any one of embodiments 21 to 28 or 37 to 67 under conditions in which the sdAb is expressed.
183. The method of embodiment 182, wherein the sdAb is a portion of an antigen-binding molecule configured as defined in any of embodiments 1 to 120
184. The method of embodiment 182 or 183, which further comprises recovering the sdAb dimer (e.g., from the host cell supernatant).
185. The method of any one of embodiments 182 to 184, wherein the cell line comprises a nucleic acid produced by the method of embodiment 177 or 178.

### 8. EXAMPLES

### 8.1. Materials and Methods

### 8.1.1. Design and Production of Anti-OX40 Stapled VHH-Fc Molecules

An in silico screen for potential locations to install cysteines to form a disulfide bond was performed between human VHs (hVH) of a homodimer (PDB:7KQY). Disulfide Bond Scan module from MOE software was used with a restricted search of only inter-chain disulfide bonds to identify cysteine substitutions that would lead to a disulfide bond. An alignment was performed between the hVH sequence and an OX40 VHH sequence (FIG. 2A) to identify VHH residues corresponding to the human VH residues identified by the MOE software. Three solutions, SS1, SS3, and SS4, were determined by structural examination. An additional solution, SS2 (Q108C, G44C, Kabat EU numbering) of hVH was included based on empirical experience on disulfide bond mediated scFv stabilization, namely VH-44 and VL-100 (Kabat EU numbering).

Two of the solutions, SS2 and SS3 were used to design the exemplary stapled anti-OX40 sdAbs depicted in FIGS. 3B and 3C. The constructs were expressed in Expi293F™ cells by transient transfection following the manufacturer's protocol (Thermo Fisher Scientific). Proteins in Expi293F™ supernatant were purified using the ProteinMaker system (Protein BioSolutions, Gaithersburg, MD) with either HiTrap™ Protein or MabSelect SuRe columns (Cytiva). After single step elution, SS3 constructs were neutralized, dialyzed into a final buffer of phosphate buffered saline (PBS) with 5% glycerol, aliquoted and stored at −80° C. until use. After elution, SS2 constructs were further purified via size exclusion chromatography (SEC) to remove high molecular weight (HMW) species.

Production details of the two anti-OX40 stapled sdAbs, OX40-VHHSS2 and OX40-VHH SS3, and the parental anti-OX40 sdAb are provided in Table E1 below.

TABLE E1

| | Molecular | Yield from 50 |
|---|---|---|
| Construct | Weight (Da) | mL CM (mg) |
| Parental anti-OX40 VHH-Fc | 76782 | 9.7 |
| OX40-VHH SS2 | 76676 | 0.7 |
| OX40-VHH SS3 | 76566 | 2.6 |

Sequences of the two anti-OX40 stapled sdAbs, OX40-VHH SS2 and OX40-VHH SS3, and the parental anti-OX40 VHH-Fc are provided in Table E2 below.

TABLE E2

| | | SEQ ID |
|---|---|---|
| Structure | Sequence | NO: |
| Parental | EVQLLESGGGEVQPGGSLRLSCAASGFTFSDAFMYWVRQAPGKE | 33 |
| anti-OX40 | REWVSSISNRGLKTAYAESVKGRFTISRDNAKNTLYLQMSSLRA | |
| VHH-Fc | EDTAVYYCSRDVDADFRGQGTLVTVKPGGGGDKTHTCPPCPAPE | |
| | LLGGPSVFLFPPKPKDTLMISRTPEVTCVVVDVSHEDPEVKENW | |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSL | |
| | TCLVKGFYPSDIAVEWESNGQPENNYKTTPPVLDSDGSFFLYSK | |
| | LTVDKSRWQQGNVFSCSVMHEALHNHYTQKSLSLSPGK | |
| Parental | EVQLLESGGGEVQPGGSLRLSCAASGFTFSDAFMYWVRQAPGKE | 34 |
| anti-OX40 | REWVSSISNRGLKTAYAESVKGRFTISRDNAKNTLYLQMSSLRA | |
| VHH | EDTAVYYCSRDVDADFRGQGTLVTVKP | |
| OX40-VHH | EVQLLESGGGEVQPGGSLRLSCAASGFTFSDAFMYWVRQAPGKC | 35 |
| SS2-Fc | REWVSSISNRGLKTAYAESVKGRFTISRDNAKNTLYLQMSSLRA | |
| | EDTAVYYCSRDVDADFRGCGTLVTVKPGGGGDKTHTCPPCPAPE | |
| | LLGGPSVFLFPPKPKDTLMISRTPEVTCVVVDVSHEDPEVKENW | |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSL | |
| | TCLVKGFYPSDIAVEWESNGQPENNYKTTPPVLDSDGSFFLYSK | |
| | LTVDKSRWQQGNVFSCSVMHEALHNHYTQKSLSLSPGK | |
| OX-VHH | EVQLLESGGGEVQPGGSLRLSCAASGFTFSDAFMYWVRQAPGKC | 36 |
| SS2 | REWVSSISNRGLKTAYAESVKGRFTISRDNAKNTLYLQMSSLRA | |
| | EDTAVYYCSRDVDADFRGCGTLVTVKP | |
| OX40-VHH | EVQLLESGGGEVQPGGSLRLSCAASGFTFSDAFMYWVRQAPGKE | 37 |
| SS3-Fc | CEWVSSISNRGLKTAYAESVKGRFTISRDNAKNTLYLQMSSLRA | |
| | EDTAVYYCSRDVDADFCGQGTLVTVKPGGGGDKTHTCPPCPAPE | |
| | LLGGPSVFLFPPKPKDTLMISRTPEVTCVVVDVSHEDPEVKENW | |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSL | |
| | TCLVKGFYPSDIAVEWESNGQPENNYKTTPPVLDSDGSFFLYSK | |
| | LTVDKSRWQQGNVFSCSVMHEALHNHYTQKSLSLSPGK | |
| OX40-VHH | EVQLLESGGGEVQPGGSLRLSCAASGFTFSDAFMYWVRQAPGKE | 38 |
| SS3 | CEWVSSISNRGLKTAYAESVKGRFTISRDNAKNTLYLQMSSLRA | |
| | EDTAVYYCSRDVDADFCGQGTLVTVKP | |

### 8.1.2. Luciferase Activity Assay

RPMI1640 media supplemented with 2 mM L-Glutamine/Penicillin/Streptomycin+10% FBS was used as the assay medium to prepare cell suspensions and dilutions of constructs.

The day of the assay, Jurkat/NFκB-Luc reporter cells were added at a density of 15,000 cells/well to 96-well white flat bottom plates. For anchorage independent experiments, HEK293/CD20 cells were added to each Jurkat/NFκB-Luc reporter cell containing well at a density of 2,500 cells/well. For anchorage dependent experiments, HEK293/a-hFc cells were added to each Jurkat/NFκB-Luc reporter cell containing well at a density of 2,500 cells/well.

Stapled sdAb and control constructs were prepared in assay media and titrated from 200 nM to 5 fM in a 4-fold dilution following a 12-point dilution range, with the 12th point, plotted at 5 fM, containing no construct protein. Titrated constructs were added to wells containing cells and plates were incubated for 5 hours at 37° C. and 5% CO₂ before the addition of 100 μL ONE-Glo™ (Promega) reagent to lyse cells and detect luciferase activity. The emitted light was captured in relative light units (RLU) on a multilabel plate reader Envision (PerkinElmer). All serial dilutions were tested in duplicates.

### 8.1.3. OX40 Binding Assay

Binding affinities of stapled sdAbs OX40-VHH SS2 and OX40-VHH SS3 and the parental construct OX40-VHH to human OX40 were evaluated at 25° C. and 37° C. Constructs were captured on a Biacore 4000 CM5 surface immobilized with anti-human Fc. His-tagged human OX40 monomer (R&D 9969-OX-050) was serially diluted 3-fold (90-0.37 nM) and injected at 30 μL/min for 5 minutes. Dissociation was monitored for 10 minutes. Kinetic parameters were evaluated by fitting the real time data using 1:1 binding model with mass transport limitation in Scrubber 2.0c.

### 8.1.4. Limited LysC Proteolysis and Native SEC-MS Analysis

Stapled sdAbs OX40-VHH SS2 and OX40-VHH SS3 and the parental construct OX40-VHH were subjected to limited LysC digestion to generate VHH and Fc fragments (arising from preferential cleavage at Lys site above the hinge region, producing VHH and Fc domains). The resulting fragments were separated using native SEC-MS (nSEC-MS). The native mass spectrum of each peak was averaged and deconvoluted using Intact Mass software from Protein Metrics and the resulting mass spectra outputs were plotted.

Similarly, stapled anti-Receptor A sdAb constructs were subjected to limited LysC digestion to generate sdVH and Fc fragments. The resulting fragments were separated using nSEC-MS. The native mass spectrum of each peak was averaged and deconvoluted using Intact Mass software from Protein Metrics and the resulting mass spectra outputs were plotted.

8.1.5. Design and Production of Anti-Receptor A Stapled sdVH-Fc Molecules

Anti-Receptor A sdVH binders were generated. To test whether sdVH-based binders can be effectively stapled, variant pairs of anti-Receptor A sdVHs were generated based on the SS2 and SS3 stapled VHH variants. Each sdVH was N-terminally fused at the N-terminus of an Fc domain, and homodimeric constructs with two sdVHs fused to an Fc region were generated. The constructs were expressed in Expi293F™ cells by transient transfection following the manufacturer's protocol (Thermo Fisher Scientific). Proteins in Expi293F™ supernatant were purified using the ProteinMaker system (Protein BioSolutions, Gaithersburg, MD) with either HiTrap™ Protein or MabSelect SuRe columns (Cytiva). After single step elution, constructs were neutralized, dialyzed into a final buffer of phosphate buffered saline (PBS) with 5% glycerol, aliquoted and stored at −80° C. until use.

Details of the parental and stapled anti-Receptor A sdAb constructs are provided in Table E3 below.

TABLE E2

| Structure | sdVHs | Staple Variant |
|---|---|---|
| AF01 | Anti-Receptor A sdVH A | Parental |
| AF02 | Anti-Receptor A sdVH B | Parental |
| AF03 | Anti-Receptor A sdVH C | Parental |
| AF05 | Anti-Receptor A sdVH D | Parental |
| AF06 | Anti-Receptor A sdVH E | Parental |
| AF07 | Anti-Receptor A sdVH F | Parental |
| AF08 | Anti-Receptor A sdVH G | Parental |
| AF09 | Anti-Receptor A sdVH H | Parental |
| AF10 | Anti-Receptor A sdVH I | Parental |
| AF11 | Anti-Receptor A sdVH J | Parental |
| AF12 | Anti-Receptor A sdVH K | Parental |
| AF13 | Anti-Receptor A sdVH L | Parental |
| AF14 | Anti-Receptor A sdVH M | Parental |
| AF15 | Anti-Receptor A sdVH A | SS2 |
| AF16 | Anti-Receptor A sdVH B | SS2 |
| AF17 | Anti-Receptor A sdVH C | SS2 |
| AF18 | Anti-Receptor A sdVH N | SS2 |
| AF19 | Anti-Receptor A sdVH D | SS2 |
| AF20 | Anti-Receptor A sdVH E | SS2 |
| AF21 | Anti-Receptor A sdVH F | SS2 |
| AF22 | Anti-Receptor A sdVH G | SS2 |
| AF23 | Anti-Receptor A sdVH H | SS2 |
| AF24 | Anti-Receptor A sdVH I | SS2 |
| AF26 | Anti-Receptor A sdVH K | SS2 |
| AF28 | Anti-Receptor A sdVH M | SS2 |
| AF29 | Anti-Receptor A sdVH A | SS3 |
| AF30 | Anti-Receptor A sdVH B | SS3 |
| AF34 | Anti-Receptor A sdVH E | SS3 |
| AF36 | Anti-Receptor A sdVH G | SS3 |
| AF41 | Anti-Receptor A sdVH L | SS3 |

### 8.2. Example 1: Agonist Activity of Anti-OX40 Stapled VHH-Fc Molecules

Two anti-OX40 stapled sdAbs were produced as described in Section 8.1.1. Anchorage-independent and -dependent agonist activities of the generated anti-OX40 stapled sdAbs were evaluated using luciferase activity assay as described in Section 8.1.2.

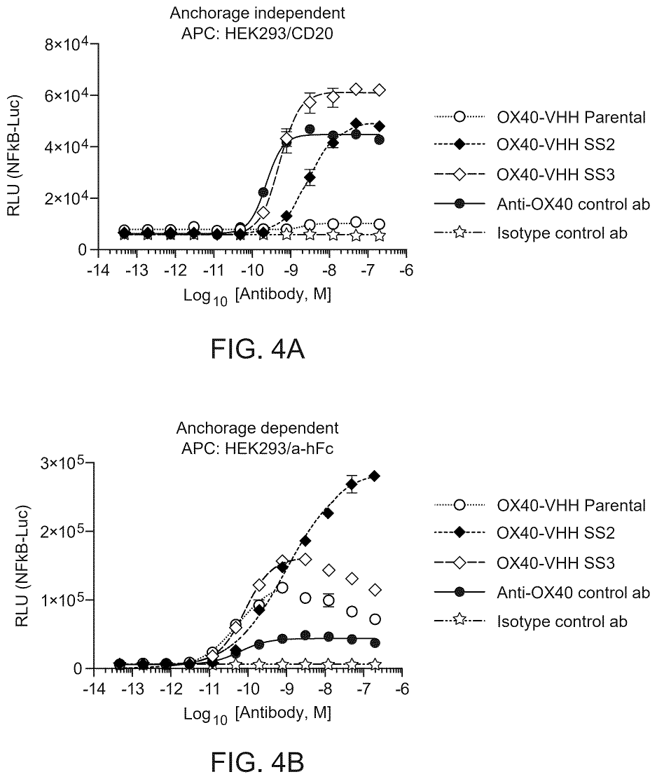

Anchorage-independent assessments revealed that both stapled sdAb constructs, OX-40-VHH SS2 and OX40-VHH SS3, displayed robust NFκB luciferase reporter activity with SS3 demonstrating higher efficacy than the anti-OX40 positive control mAb, whereas the parental molecule displayed little to no reporter activity (FIG. 4A). These results demonstrated that dimerization-enabling modifications of the VHH domains of a non-agonist parental anti-OX40 VHH-Fc construct were sufficient to transform the resulting constructs into OX40 agonists. When the same constructs were evaluated with anchorage-dependent assessments, the highest levels of NFκB luciferase reporter activity were observed with the two stapled sdAbs, OX-40-VHH SS2 and OX40-VHH SS3 (FIG. 4B).

### 8.3. Example 2: Binding Kinetics of Anti-OX40 Stapled VHH-Fc Molecules

Binding affinity of stapled sdAbs OX40-VHH SS2 and OX40-VHH SS3 to human OX40 was evaluated as described in Section 8.1.3.

Results are summarized in Table E3. Briefly, both anti-OX40 stapled sdAbs, OX40-VHH SS2 and OX40-VHH SS3, displayed binding affinities comparable to those observed with the parental control construct. These observations suggest that the modifications of the two stapled sdAbs had minimal impact on their binding affinity to monomeric hOX40.

TABLE E3

| | | | 90 nM | | | | |
|---|---|---|---|---|---|---|---|
| | T | | hOX40.His | ka | kd | KD | t1/2 |
| Construct | (° C.) | mAb Capture | Bound (RU) | (1/Ms) | (1/s) | (M) | (min) |
| parental | 25 | 211.0 ± 1.6 | 58.8 | 3.00E+05 | 2.56E−02 | 8.52E−08 | 0.5 |
| anti-OX40 | 37 | 258.8 ± 1.4 | 50.3 | 5.80E+05 | 4.76E−02 | 8.21E−08 | 0.2 |
| VHH-Fc | | | | | | | |
| OX-40-VHH | 25 | 234.6 ± 1.1 | 33.2 | 2.12E+05 | 2.71E−02 | 1.28E−07 | 0.4 |
| SS2 | 37 | 273.1 ± 1.6 | 27.9 | 3.89E+05 | 6.66E−02 | 1.71E−07 | 0.2 |
| OX-40-VHH | 25 | 357.7 ± 8.7 | 68.4 | 1.66E+05 | 1.61E−02 | 9.67E−08 | 0.7 |
| SS3 | 37 | 415.3 ± 1.7 | 57.6 | 2.82E+05 | 3.93E−02 | 1.39E−07 | 0.3 |
| Isotype | 25 | 357.7 ± 8.7 | −0.6 | NB | NB | NB | NB |
| control | 37 | 415.3 ± 1.7 | 0.0 | NB | NB | NB | NB |
| antibody | | | | | | | |

### 8.4. Example 3: Evaluation of VHH Dimerization of Anti-OX40 Stapled VHH-Fc Molecules

LysC can digest human IgG1 (hIgG1) Fc-based constructs above the hinge region. Therefore, anti-OX40 sdAb constructs were subjected to limited LysC digestion to produce Fc and VHH construct fragments, which were then evaluated using nSEC-MS as described in Section 8.1.4.

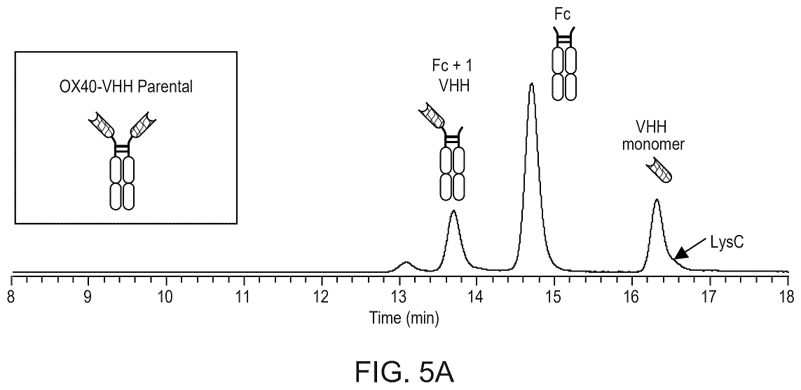

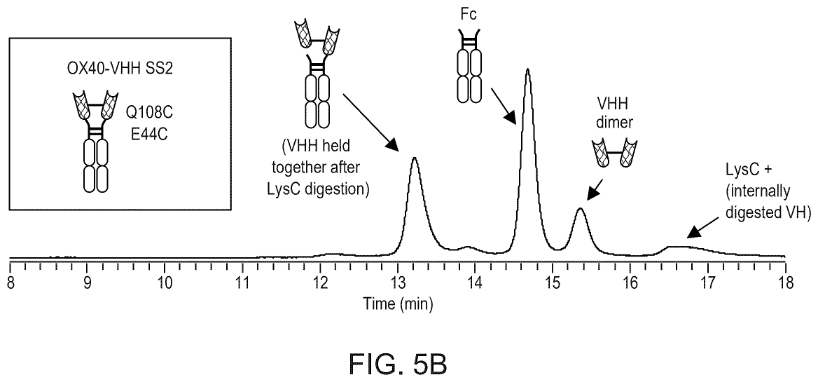

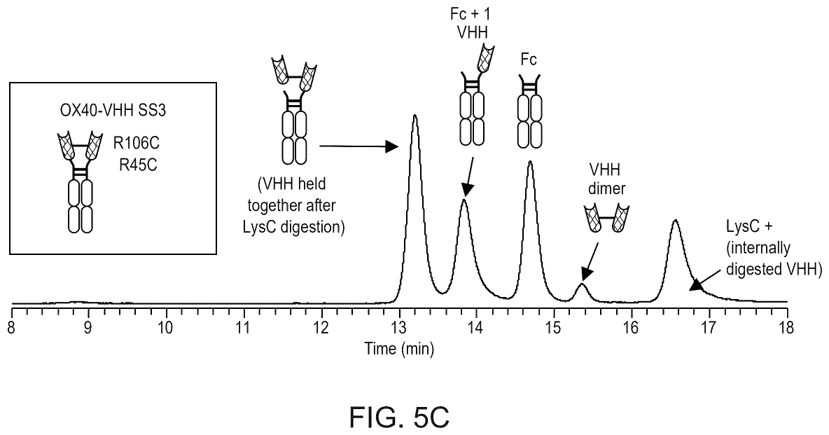

Three main peaks were observed with the parental anti-OX40 sdAb construct. The heaviest of these peaks corresponded to the molecular weight of a VHH-Fc structure consisting of a single VHH arm, suggesting proteolysis of one LysC target site and resulting in free VHH monomer. The second peak corresponded to free Fc regions. The third peak corresponded to free VHH monomers (FIG. 5A). There was no evidence of dimerized VHH domains in the parental construct.

The heaviest peak observed with the anti-OX40 stapled sdAb construct OX40-VHH SS2 corresponded to a structure that had two VHH domains and an Fc region (FIG. 5B), likely entailing structures with only one LysC target site successfully proteolyzed, but the freed VHH domain was still attached to the construct due to covalent dimerization with the other VHH domain. The peak for VHH monomers was dramatically reduced relative to the parental construct and there was an additional peak corresponding to VHH dimers, suggesting that LysC digestion resulted in VHH pairs that remained as dimers.

Evaluation of the anti-OX40 stapled sdAb construct OX40-VHH SS3 resulted in peaks that correspond to structures with dimerized VHH domains as well as non-dimerized (monomeric) VHH domains, suggesting that the VHH dimerize non-covalently (FIG. 5C).

VHH dimerization and the distance between the two VHH domains of the two constructs were further evaluated using in silico structural models constructed using cryoEM images.

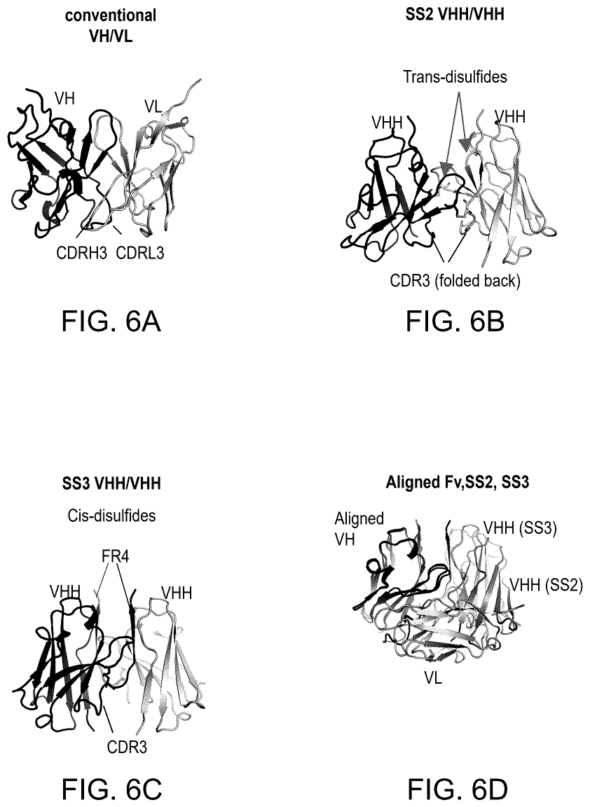

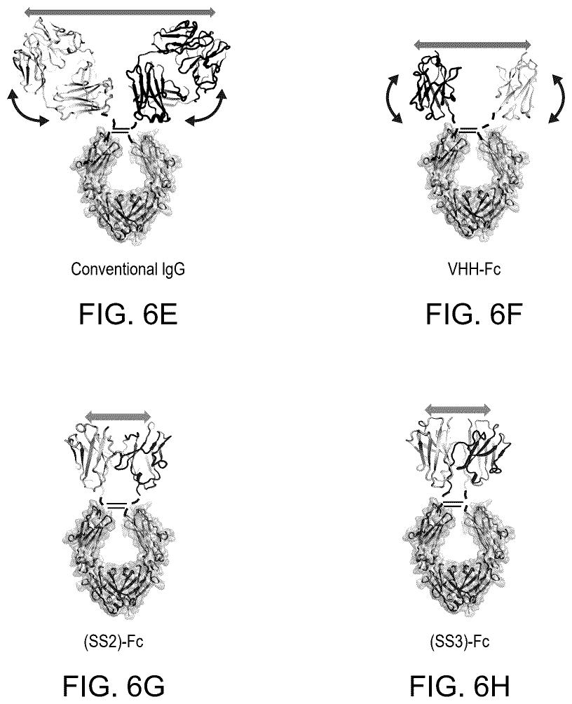

The structure of a conventional VH-VL pair is shown in FIG. 6A. The cryoEM structure depicts dimerization of SS2 through inter-VHH (trans-disulfide) crosslinking between cysteine at position Q108C (Kabat EU numbering) of one VHH and the cysteine at position G44C (Kabat EU numbering) of the other VHH (FIG. 6B). The cryoEM structure of SS3 shows formation of intra-VHH (cis-disulfide) crosslinking between cysteines at positions L45C and W106C (Kabat EU numbering) of each VHH, and FR4 of each VHH extending towards the other VHH and interacting to form a VHH dimer (FIG. 6C). The structural alignment of the unstapled, SS2, and SS3 VHHs in FIGS. 6A-6C are shown in FIG. 6D.

Modeling of sdAb constructs comprising either SS2 or SS3 VHH domains revealed a shortening of the distance between the two VHH domains (FIGS. 6G and 6H, respectively) relative to a conventional IgG antibody structure (FIG. 6E) as well as a non-stapled VHH antibody structure (FIG. 6F).

### 8.5. Example 4: Evaluation of VHH Dimerization of Anti-Receptor A Stapled sdVH-Fc Molecules

Anti-Receptor A constructs having two anti-Receptor A sdVHs either in parental, SS2, or SS3 configurations were designed and produced as described in Section 6.1.5, and subjected to limited LysC digestion to produce Fc and sdVH fragments. LysC digested sdVH constructs and undigested sdVH constructs were then evaluated using nSEC-UV/MS as described in Section 6.1.4.

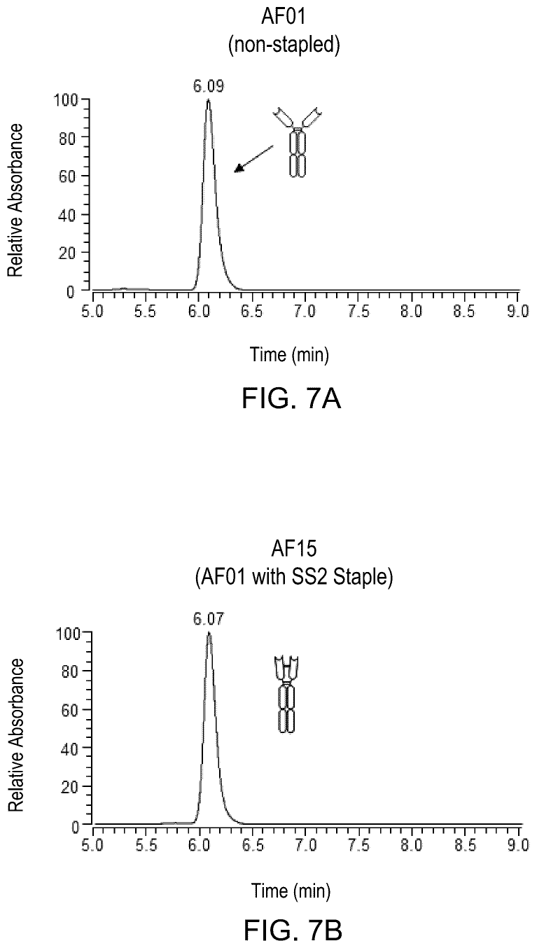

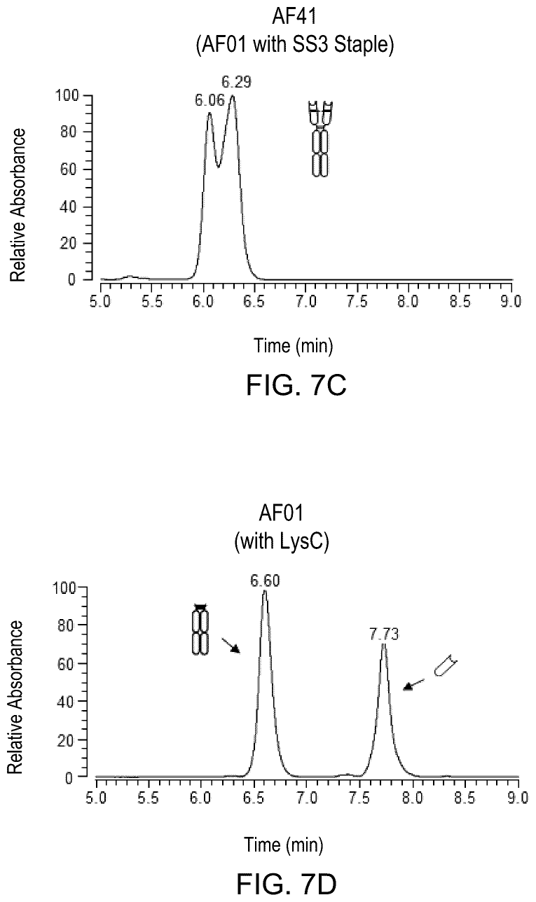

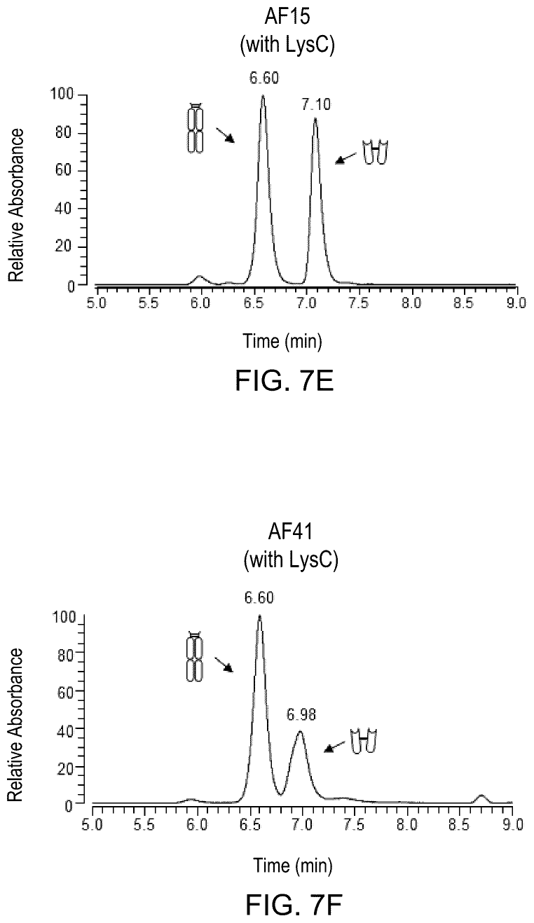

First, the elution profiles of AF01 (an unstapled anti-Receptor A sdAb construct), AF15 (an SS2-stapled anti-Receptor A sdAb construct), and AF41 (an SS3-stapled anti-Receptor A sdAb construct) were evaluated. FIGS. 7A-7C show elution profiles of undigested constructs, and FIGS. 7D-7F show elution profiles of digested products. The results of this comparison revealed that digestion of the SS2 and SS3 constructs generated sdVH dimers and Fc fragments, while the parental constructs generated sdVH monomers and Fc fragments.

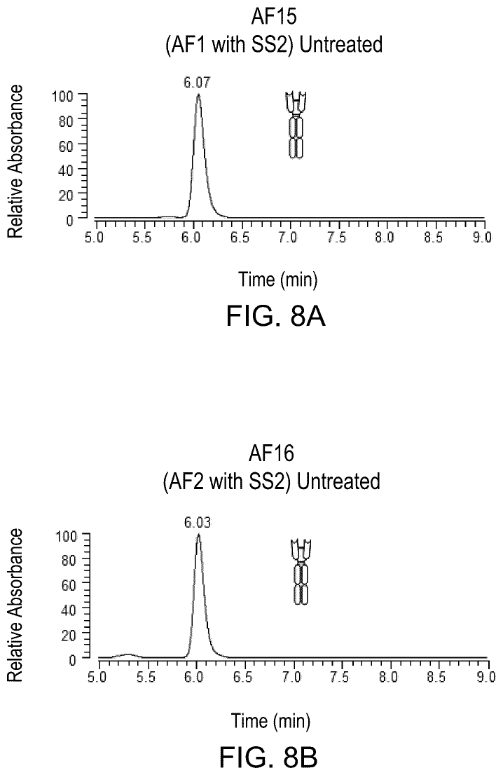

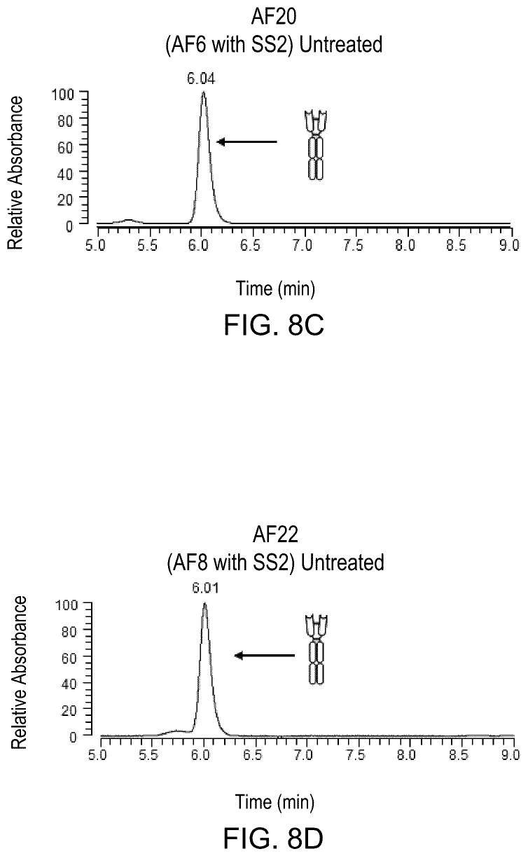

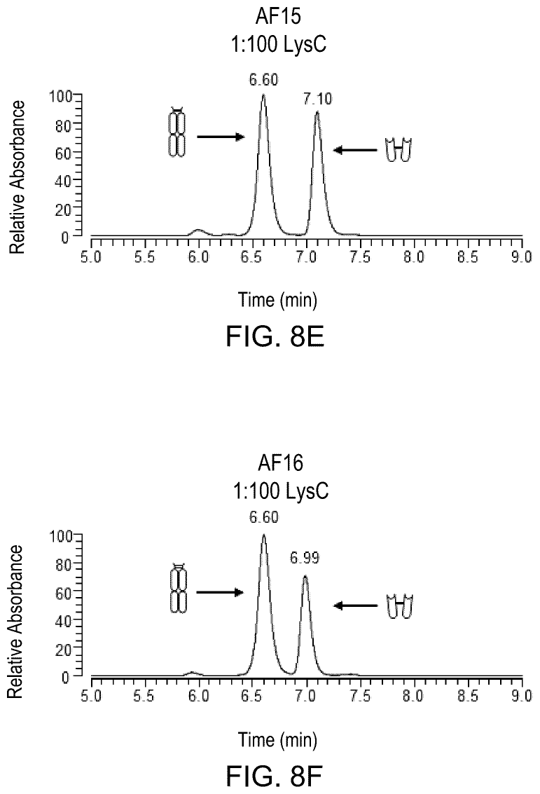

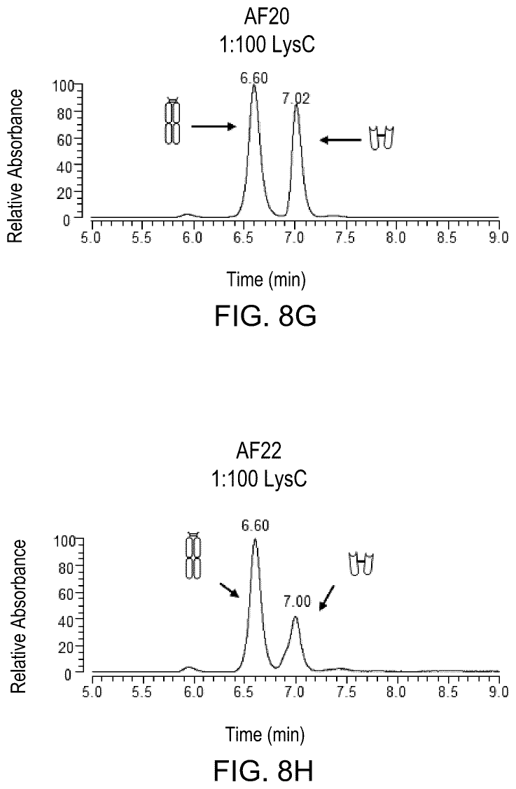

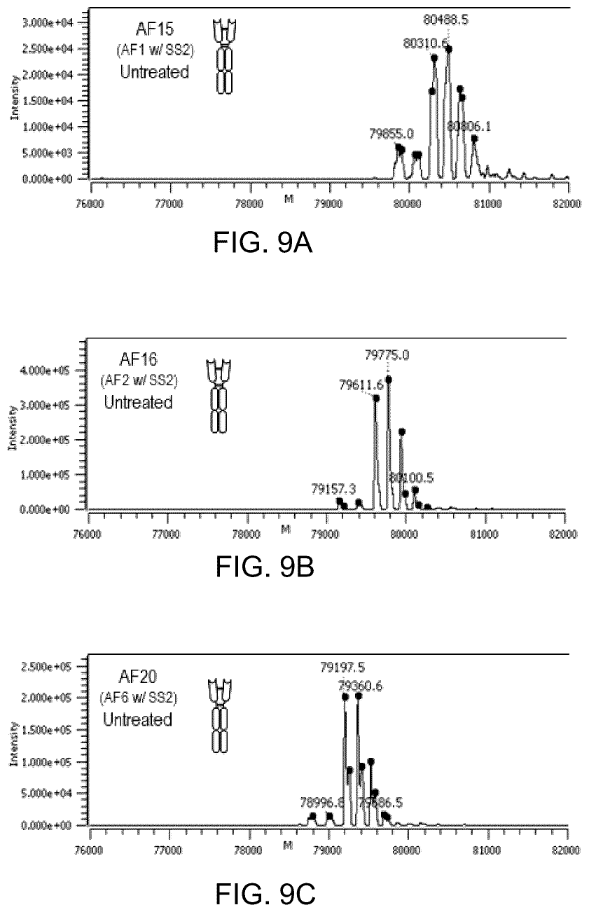

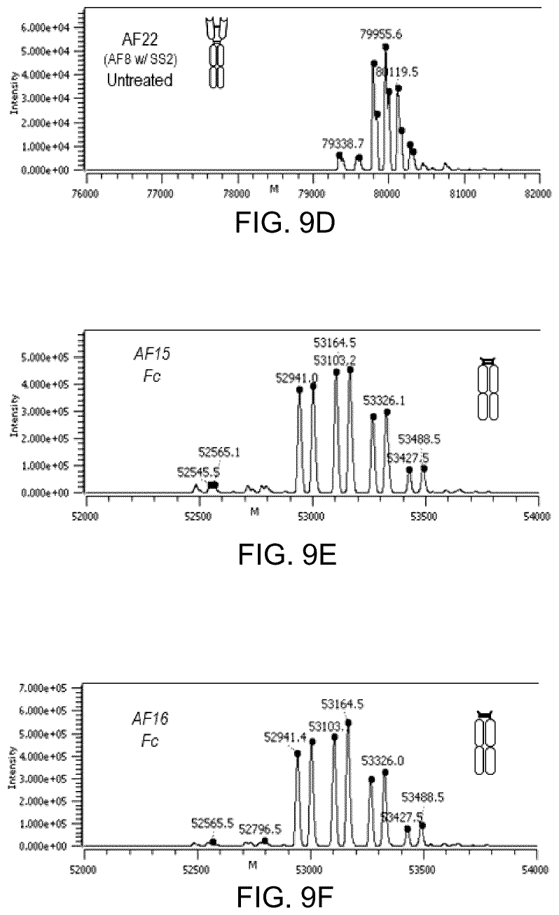

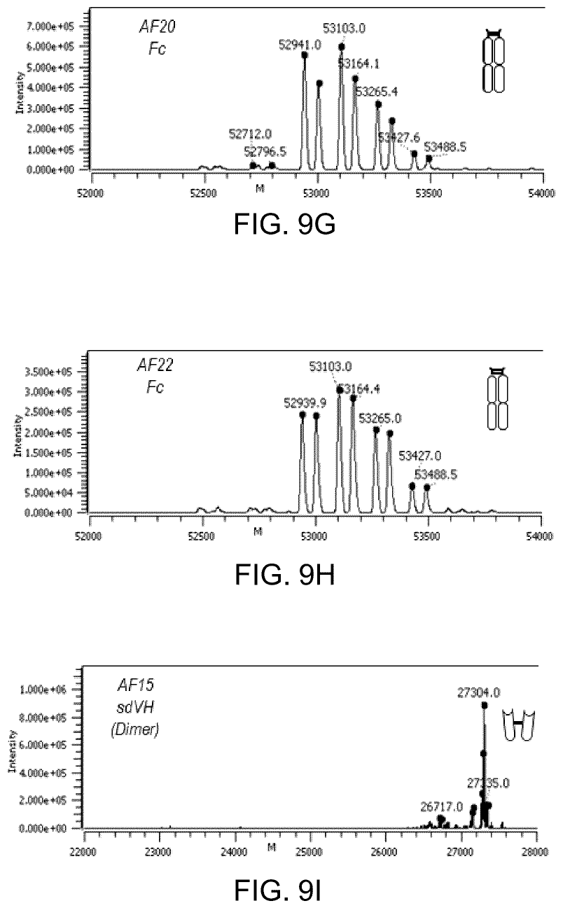

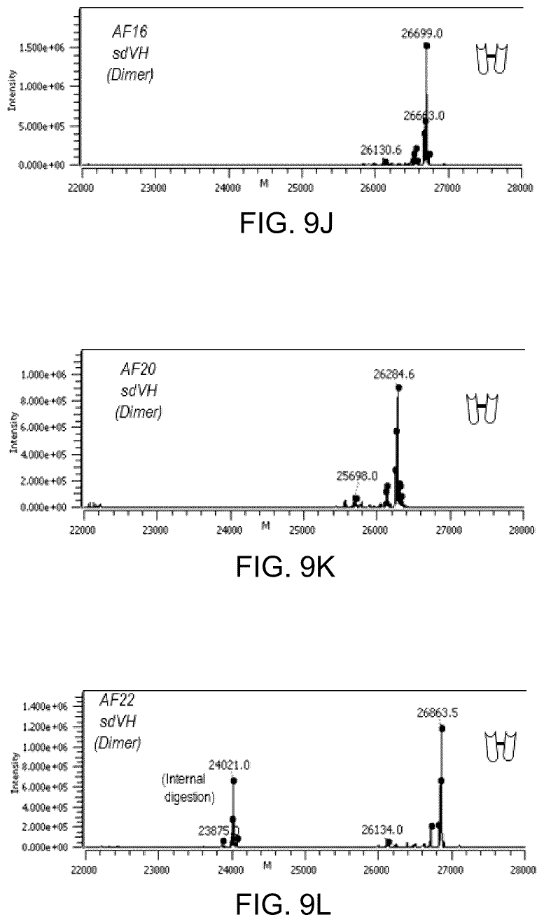

Four SS2-stapled anti-Receptor A sdAb constructs, AF15, AF16, AF20, and AF22, were evaluated with and without LysC digestion using nSEC-UV/MS. All four sdVH constructs were associated with similar elution profiles with two peaks, one corresponding to the Fc fragment and the other peak corresponding to an sdVH dimer (FIGS. 8A-8H). Accurate intact mass analysis of the four anti-Receptor A sdAb constructs that were untreated or LysC treated confirmed formation of covalent stapled sdVHs (FIGS. 9A-9L).

An in-depth nSEC-UV/MS analysis was performed with thirteen unstapled (AF01, AF02, AF03, AF05, AF06, AF07, AF08, AF09, AF10, AF11, AF12, AF13, and AF14) and twelve SS2-stapled anti-Receptor A sdAb constructs (AF15, AF16, AF17, AF18, AF19, AF20, AF21, AF22, AF23, AF24, AF26, and AF28) that were untreated or digested with LysC. Consistent with the above observations, the elution profiles of the SS2 stapled constructs showed that sdVH dimers were formed by LysC digestion, whereas the elution profiles of the unstapled constructs showed that sdVH monomers were formed. Further analysis of mass spectra of SS2-stapled anti-Receptor A sdAb constructs confirmed formation of disulfide-bonded dimers in human single domain VHs although results with AF18 and AF28 indicate potential formation of non-covalent dimers (data not shown).

The ability of a subset of stapled anti-Receptor A sdAb constructs to activate signaling through Receptor A was tested, and it was found that, despite successful dimerization, the tested constructs did not induce increased Receptor A signaling relative to the unstapled parental constructs (data not shown). This may have been due to reduced binding of Receptor A by the stapled constructs relative to parental constructs. Structural modeling indicated that for the specific Receptor A binders tested, the stapled constructs may have been sterically blocked from optimal binding and activation of Receptor A. This indicates that structural modeling (e.g., in silico structural modeling using cryoEM images) can be used to identify suitable sdAb candidates for stapling by evaluating whether stapling of sdAb arms may impact target binding.

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1. An antigen-binding molecule comprising:
(a) a first polypeptide chain comprising:
(i) a first single domain antibody (sdAb) variable domain comprising a first cysteine residue; and
(ii) a first Fc domain; and
(b) a second polypeptide chain comprising:
(i) a second sdAb variable domain comprising a second cysteine residue; and
(ii) a second Fc domain associated with the first Fc domain;
wherein the first cysteine residue and the second cysteine residue are substitutions of non-cysteine amino acids in a reference sdAb sequence; and
wherein the first cysteine residue is at a position in the first sdAb variable domain that differs from the position of the second cysteine residue in the second sdAb variable domain.

2. (canceled)

3. The antigen-binding molecule of claim 1, wherein the first sdAb variable domain comprises a framework region 1 (FR1), a framework region 2 (FR2), a framework region 3 (FR3), and a framework region 4 (FR4) and the second sdAb variable domain comprises an FR1, an FR2, an FR3, and an FR4; wherein the first cysteine residue is in FR4 of the first sdAb variable domain; and wherein the second cysteine residue is in FR2 of the second sdAb variable domain.

4. The antigen-binding molecule of claim 3, wherein the first sdAb variable domain further comprises a third cysteine residue, wherein the third cysteine residue is a substitution of a non-cysteine amino acid in a reference sdAb sequence and is at the same relative position in the first sdAb variable domain as the position of the second cysteine in the second sdAb variable domain; wherein the second sdAb variable domain further comprises a fourth cysteine residue, wherein the fourth cysteine residue is a substitution of a non-cysteine amino acid in a reference sdAb sequence and is at the same relative position in the second sdAb variable domain as the position of the first cysteine in the first sdAb variable domain.

5. The antigen-binding molecule of claim 4, further comprising: (A) a first intra-chain disulfide bond between the first cysteine residue and the third cysteine residue; and (B) a second intra-chain disulfide bond between the second cysteine residue and the fourth cysteine residue; wherein the first sdAb and the second sdAb have a stronger inter-chain association as compared to a reference pair of sdAbs lacking the first, second, third and/or fourth cysteine residues.

6. (canceled)

7. The antigen-binding molecule of claim 3, wherein the FR4 of the first sdAb variable domain associates with the second sdAb variable domain and wherein the FR4 of the second sdAb variable domain associates with the first sdAb variable domain.

8. The antigen-binding molecule of claim 5, wherein the first cysteine residue is at a position corresponding to W106 of a human VH, numbering according to Kabat; wherein the second cysteine residue is at a position corresponding to L45 of a human VH, numbering according to Kabat.

9. The antigen-binding molecule of claim 5, wherein the first sdAb variable domain is a first human sdVH and the second sdAb variable domain is a second human sdVH; wherein the first cysteine residue is at position W106 of the first human sdVH and the second cysteine residue is at position L45 of the second human sdVH, numbering according to Kabat; and wherein the third cysteine residue is at position L45 of the first human sdVH and the fourth cysteine residue is at position W106 of the second human sdVH, numbering according to Kabat.

10. The antigen-binding molecule of claim 5, wherein the first sdAb variable domain is a first VHH and the second sdAb variable domain is a second VHH; wherein the first cysteine residue is at position R106 of the first VHH and the second cysteine residue is at position R45 of the second VHH, numbering according to Kabat; and wherein the third cysteine residue is at position R45 of the first VHH and the fourth cysteine residue is at position R106 of the second VHH, numbering according to Kabat.

11. The antigen-binding molecule of claim 1, further comprising an inter-chain disulfide bond between the first cysteine residue and the second cysteine residue; wherein the first cysteine residue is at a position corresponding to Q108 of a human VH, numbering according to Kabat; and wherein the second cysteine residue is at a position corresponding to G44 of a human VH, numbering according to Kabat.

12. The antigen-binding molecule of claim 11, wherein the first sdAb variable domain is a first human sdVH and the second sdAb variable domain is a second human sdVH, wherein the first cysteine residue is at position Q108 of the first human sdVH and the second cysteine residue is at position G44 of the second human sdVH, numbering according to Kabat; and wherein the third cysteine residue is at position G44 of the first human sdVH and the fourth cysteine residue is at position Q108 of the second human sdVH, numbering according to Kabat.

13. The antigen-binding molecule of claim 11, wherein the first sdAb variable domain is a first VHH and the second sdAb variable domain is a second VHH; wherein the first cysteine residue is at position Q108 of the first VHH and the second cysteine residue is at position E44 of the second VHH, numbering according to Kabat; and wherein the third cysteine residue is at position E44 of the first VHH and the fourth cysteine residue is at position Q108 of the second VHH, numbering according to Kabat.

14. An antigen-binding molecule comprising:
(a) a first polypeptide chain comprising:
(i) a first single domain antibody (sdAb) variable domain comprising a first cysteine residue; and
(ii) a first Fc domain; and
(b) a second polypeptide chain comprising:
(i) a second sdAb variable domain comprising a second cysteine residue covalently bonded to the first cysteine residue; and
(ii) a second Fc domain associated with the first Fc domain.

15.-18. (canceled)

19. The antigen-binding molecule of claim 14, wherein:
(a) the first sdAb variable domain is a first human sdVH and the second sdAb variable domain is a second human sdVH; wherein the first cysteine residue is at position Q108 of the first human sdVH and the second cysteine residue is at position G44 of the second human sdVH, numbering according to Kabat; and wherein the third cysteine residue is at position G44 of the first human sdVH and the fourth cysteine residue is at position Q108 of the second human sdVH, numbering according to Kabat; or
(b) the first sdAb variable domain is a first VHH and the second sdAb variable domain is a second VHH; wherein the first cysteine residue is at position Q108 of the first VHH and the second cysteine residue is at position E44 of the second VHH, numbering according to Kabat; and wherein the third cysteine residue is at position E44 of the first VHH and the fourth cysteine residue is at position Q108 of the second VHH, numbering according to Kabat.

20. (canceled)

21. An antigen-binding molecule comprising:
(a) a first polypeptide chain comprising a first sdAb variable domain comprising an FR1, an FR2, an FR3, and an FR4, wherein the FR4 comprises a first cysteine residue, wherein the first cysteine residue is a substitution of a non-cysteine amino acid in a reference FR4 sequence; and
(b) a second polypeptide chain comprising a second sdAb variable domain comprising an FR1, an FR2, an FR3, and an FR4, wherein the FR2 comprises a second cysteine residue, wherein the second cysteine residue is a substitution of a non-cysteine amino acid in a reference FR2 sequence.

22. The antigen-binding molecule of claim 21, wherein the first sdAb variable domain further comprises a third cysteine residue, wherein the third cysteine residue is a substitution of a non-cysteine amino acid in a reference sdAb sequence and is at the same relative position in the first sdAb variable domain as the position of the second cysteine in the second sdAb variable domain; and wherein the second sdAb variable domain further comprises a fourth cysteine residue, wherein the fourth cysteine residue is a substitution of a non-cysteine amino acid in a reference sdAb sequence and is at the same relative position in the second sdAb variable domain as the position of the first cysteine in the first sdAb variable domain.

23. The antigen-binding molecule of claim 22, further comprising: (A) a first intra-chain disulfide bond between the first cysteine residue and the third cysteine residue; and (B) a second intra-chain disulfide bond between the second cysteine residue and the fourth cysteine residue; wherein the first sdAb and the second sdAb have a stronger inter-chain association as compared to a reference pair of sdAbs lacking the first, second, third and/or fourth cysteine residues; wherein the FR4 of the first sdAb variable domain associates with the second sdAb variable domain and wherein the FR4 of the second sdAb variable domain associates with the first sdAb variable domain.

24. (canceled)

25. (canceled)

26. The antigen-binding molecule of claim 23, wherein:
(a) the first sdAb variable domain is a first human sdVH and the second sdAb variable domain is a second human sdVH; wherein the first cysteine residue is at position W106 of the first human sdVH and the second cysteine residue is at position L45 of the second human sdVH, numbering according to Kabat; and wherein the third cysteine residue is at position L45 of the first human sdVH and the fourth cysteine residue is at position W106 of the second human sdVH, numbering according to Kabat; or
(b) the first sdAb variable domain is a first VHH and the second sdAb variable domain is a second VHH; wherein the first cysteine residue is at position R106 of the first VHH and the second cysteine residue is at position R45 of the second VHH, numbering according to Kabat; and wherein the third cysteine residue is at position R45 of the first VHH and the fourth cysteine residue is at position R106 of the second VHH, numbering according to Kabat.

27. (canceled)

28. The antigen-binding molecule of claim 22, further comprising an inter-chain disulfide bond between the first cysteine residue and the second cysteine residue; wherein the first cysteine residue is at a position corresponding to Q108 of a human VH, numbering according to Kabat; and wherein the second cysteine residue is at a position corresponding to G44 of a human VH, numbering according to Kabat;
wherein:
(a) the first sdAb variable domain is a first human sdVH and the second sdAb variable domain is a second human sdVH; wherein the first cysteine residue is at position Q108 of the first human sdVH and the second cysteine residue is at position G44 of the second human sdVH, numbering according to Kabat; and wherein the third cysteine residue is at position G44 of the first human sdVH and the fourth cysteine residue is at position Q108 of the second human sdVH, numbering according to Kabat; or
(b) the first sdAb variable domain is a first VHH and the second sdAb variable domain is a second VHH; wherein the first cysteine residue is at position Q108 of the first VHH and the second cysteine residue is at position E44 of the second VHH, numbering according to Kabat; and wherein the third cysteine residue is at position E44 of the first VHH and the fourth cysteine residue is at position Q108 of the second VHH, numbering according to Kabat.

29.-36. (canceled)

45. The antigen-binding molecule of claim 1, wherein the first sdAb variable domain and the second sdAb variable domain each have specific affinity for a target molecule, wherein the target molecule is a component of a membrane-bound receptor that comprises multiple copies of the target molecule.

46. (canceled)

47. The antigen-binding molecule of claim 45, wherein the target molecule is OX40, the first sdAb variable domain is a first VHH, and the second sdAb variable domain is a second VHH; and wherein:
(a) the first VHH comprises a Q108C amino acid substitution, numbering according to Kabat, and an amino acid sequence having at least 90% sequence identity to the amino acid sequence of SEQ ID NO:36, and the second VHH comprises an E44C amino acid substitution, numbering according to Kabat, and an amino acid sequence having at least 90% sequence identity to the amino acid sequence of SEQ ID NO:36;
(b) the first polypeptide chain comprises a Q108C amino acid substitution in the first VHH, numbering according to Kabat, and an amino acid sequence having at least 90% sequence identity to the amino acid sequence of SEQ ID NO:35 and the second polypeptide chain comprises an E44C amino acid substitution in the second VHH, numbering according to Kabat, and an amino acid sequence having at least 90% sequence identity to the amino acid sequence of SEQ ID NO:35;
(c) the first VHH comprises an R45C and an R106C amino acid substitution, numbering according to Kabat, and amino acid sequence having at least 90% sequence identity to the amino acid sequence of SEQ ID NO:38, and the second VHH comprises an R45C and an R106C amino acid substitution, numbering according to Kabat, and amino acid sequence having at least 90% sequence identity to the amino acid sequence of SEQ ID NO: 38; or
(d) the first polypeptide chain comprises an R45C and an R106C amino acid substitution in the first VHH and amino acid sequence having at least 90% sequence identity to the amino acid sequence of SEQ ID NO:37, and the second polypeptide chain comprises an R45C and an R106C amino acid substitution in the second VHH and amino acid sequence having at least 90% sequence identity to the amino acid sequence of SEQ ID NO: 37.

48.-55. (canceled)

56. A host cell engineered to express the antigen-binding molecule of claim 1.

57. (canceled)

58. A method of producing the antigen-binding molecule of claim 1, comprising culturing the host cell of claim 56 and recovering the antigen-binding molecule expressed thereby.

59. A pharmaceutical composition comprising the antigen-binding molecule of claim 1 and an excipient.

60. A method of agonizing a target molecule comprising contacting the target molecule with the molecule of claim 1 in which the first and/or second sdAb bind to the target molecule.

61. (canceled)

62. (canceled)

63. A method of activating an immune response in a subject in need thereof, the method comprising administering to the subject the antigen-binding molecule of claim 1.

64.-67. (canceled)

68. A method for forming a dimer of sdAb moieties within an antigen-binding molecule, the method comprising co-expressing a first polypeptide chain and a second polypeptide chain within a cell, wherein:
(a) the first polypeptide chain comprises a first sdAb variable domain comprising an FR1, an FR2, an FR3, and an FR4, wherein
(i) the FR4 comprises a first cysteine residue, which is a substitution of a non-cysteine amino acid in a reference FR4 sequence; and
(ii) a third cysteine residue, which is a substitution of a non-cysteine amino acid in a reference FR2 sequence; and
(b) the second polypeptide chain comprises a second sdAb variable domain comprising an FR1, an FR2, an FR3, and an FR4, wherein:
(i) the FR2 comprises a second cysteine residue, which is a substitution of a non-cysteine amino acid in a reference FR2 sequence; and
(ii) a fourth cysteine residue, which is a substitution of a non-cysteine amino acid in a reference FR4 sequence;
wherein the third cysteine residue is at the same relative position in the first sdAb variable domain as the position of the second cysteine residue in the second sdAb variable domain; and
wherein the fourth cysteine residue is at the same relative position in the second sdAb variable domain as the position of the first cysteine residue in the first sdAb variable domain.

69.-71. (canceled)
